SEQUENCE LISTING (141 sequences)

SEQ ID NO: 1 - DNA, 1290 bp, Synthetic construct
  source 1..1290: mol_type = other DNA; note = Codon-optimized human alpha-galactosidase A nucleotide sequence #1 (hGLAco1)
atgcagctgaggaaccctgagctgcacctgggctgtgccctggccctgaggttcctggccctggtgagctgggacatccccggggctagagccctggacaatggcctggctaggacccccaccatgggctggctgcactgggagaggttcatgtgcaacctggactgccaagaggagcctgacagctgcatcagtgagaagctgttcatggagatggctgagctgatggtgtcagagggctggaaggatgccggctatgagtacctgtgcattgatgactgctggatggcccctcagagagactcagaaggcagactgcaagctgaccctcagaggttcccccatggcatcaggcagctggccaactatgtgcacagcaagggcctgaagctgggcatctatgcagatgtgggcaacaagacctgtgccggcttccccggcagctttggctactatgacattgatgcccagacctttgctgactggggagtggacctgctgaagtttgatggctgctactgtgacagcctggagaacctggcagatggctacaagcacatgagcctggccctgaacaggaccggcagaagcattgtgtacagctgtgagtggcccctgtacatgtggccctttcagaagcccaactacacagagatcagacagtactgcaaccactggaggaactttgctgacattgatgacagctggaagagcatcaagagcatcctggactggacaagcttcaaccaagagaggattgtggatgtggccggccccggcggctggaatgaccctgacatgctggtgattggcaactttggcctgagctggaatcagcaagtgacacagatggccctgtgggccatcatggctgcccccctgttcatgagcaatgacctgaggcacatcagcccccaagccaaggccctgctgcaagacaaggatgtgattgccatcaaccaagaccccctgggcaagcaaggctatcagctgaggcaaggagacaactttgaagtgtgggagagacccctgagcggcctggcctgggctgtggccatgatcaacaggcaagagattggcggccctagaagctacaccattgctgtggctagcctgggcaagggagtggcctgcaaccctgcctgcttcatcacacagctgctgcctgtgaagaggaagctgggcttctatgagtggacaagcaggctgagatcacacatcaaccccaccggcactgtgctgctgcagctggagaacaccatgcagatgagcctgaaggacctgctgtga

SEQ ID NO: 2 - DNA, 1290 bp, Synthetic construct
  source 1..1290: mol_type = other DNA; note = Codon-optimized human alpha-galactosidase A nucleotide sequence #2 (hGLAco2)
atgcagctgaggaacccagagctgcacctgggctgtgccctggccctcaggtttctggccctggtgtcctgggacatccctggggccagagccctggacaatggcctggctagaacccctaccatgggctggctgcactgggagcggttcatgtgcaacctggactgccaggaggaaccagactcctgcatctctgagaagctgttcatggagatggcagagctgatggtctctgagggatggaaggatgccggctatgagtacctgtgcattgatgactgctggatggcccctcagcgggactctgaaggcagactgcaggcagaccctcagcggttccctcatggcatccggcagctggctaactatgtgcacagcaagggcctgaagctgggaatctatgcagatgtgggcaacaagacctgtgccggattccctggctcttttggatattatgacattgatgcccagacctttgctgattggggagtggacctgctgaagtttgatggctgctactgtgatagcctggaaaacctggctgatggctacaagcacatgagcctggccctgaaccggacaggcagaagcattgtgtacagctgtgagtggccactgtacatgtggccattccagaagcccaactacacagagatcagacagtactgcaaccactggcggaattttgcagatattgatgacagctggaagtccatcaagagcatcctggactggaccagcttcaaccaagaaagaattgtggatgtggccggccctggcggctggaatgatccagatatgctggtcattggcaactttggcctgagctggaaccagcaggtgacccagatggccctgtgggccatcatggctgcccctctgtttatgagcaatgacctgagacacatcagccctcaggccaaggccctgctgcaggacaaggatgtgattgcaatcaaccaggaccctctgggcaaacagggctatcagctgagacaaggggacaactttgaggtgtgggaaagacctctgagcggcctggcttgggctgtggccatgatcaatagacaggagattggcggacctagatcctacaccattgctgtggccagcctgggcaagggagtggcttgtaatcctgcctgttttatcacccagctgctccctgttaagagaaagctgggcttctatgagtggaccagccggctgagatctcacatcaaccctacaggcacagtgctgctgcagctggaaaacaccatgcagatgagcctgaaagacctgctgtga

SEQ ID NO: 3 - DNA, 59 bp, Synthetic construct
  source 1..59: mol_type = other DNA; note = Short hepatocyte specific promoter (sH) nucleotide sequence
ggcaaagtccatattgacttaggttaatcattaactctttgttgactaagtcaataatc

SEQ ID NO: 4 - DNA, 185 bp, Synthetic construct
  source 1..185: mol_type = other DNA; note = Hepatocyte-cardiomyocyte bi-specific promoter HC1 nucleotide sequence
ggcaaagtccatattgacttaggttaatcattaactctttgttgactaagtcaataatcggggttatttttaaactatctgctcatctacttccggtctttccattcaggaatgcctttcttgggggggcggggctataaaagccccaggctgggagcagccatcacagaagtccactcattctt

SEQ ID NO: 5 - DNA, 216 bp, Synthetic construct
  source 1..216: mol_type = other DNA; note = Hepatocyte-cardiomyocyte bi-specific promoter HC2 nucleotide sequence
ggcaaagtccatattgacttaggttaatcattaactctttgttgactaagtcaataatcctgtggaatgtgaggcctggcctgggagatatttttgctgcactttgagccaccccgccccctggaactcagaccctgcacagtccatgccataacgggggcggggctataaaagccccaggctgggagcagccatcacagaagtccactcattctt

SEQ ID NO: 6 - DNA, 256 bp, Synthetic construct
  source 1..256: mol_type = other DNA; note = Hepatocyte-cardiomyocyte bi-specific promoter HC3 nucleotide sequence
ggttaaaaataacactatctgctcatcaggaggaatgtgcctaaaaaaagggacctttcttgggctcctcctgtcacctccagagccaaaggaggaggagccactccaaatttagaggcaaagtccatattgacttaggttaatcattaactctttgttgactaagtcaataatcagaatcagcaggtttgcagtcagattggcagtataaaagccccaggctgggagcagccatcacagaagtccactcattctt

SEQ ID NO: 7 - DNA, 308 bp, Synthetic construct
  source 1..308: mol_type = other DNA; note = Hepatocyte-cardiomyocyte bi-specific promoter HC4 nucleotide sequence
ggttaaaaataactgaggtaagggccatggcagggtgggagaggcggtgtgagaaggtcctgtcttcccactatctgctcatcagccctttgaaggggaggaatgtgcccaaggactaaaaaaaggccgtggagccagagaggctggggcagcagacctttcttgggcggcaaagtccatattgacttaggttaatcattaactctttgttgactaagtcaataatcagaatcagcaggtttgcagtcagattggcagtataaaagccccaggctgggagcagccatcacagaagtccactcattctt

SEQ ID NO: 8 - DNA, 276 bp, Synthetic construct
  source 1..276: mol_type = other DNA; note = Hepatocyte-cardiomyocyte bi-specific promoter HC5 nucleotide sequence
ggaccacatgggcttatatggcgtggggtacatgttcctgtagccttgtccctggcacctgccaaaatagcagccaacaccccccacccccaccgccatccccctgccccacccgtcccctgtcgcacattcctccggcaaagtccatattgacttaggttaatcattaactctttgttgactaagtcaataatcagaatcagcaggtttgcagtcagattggcagtataaaagccccaggctgggagcagccatcacagaagtccactcattctt

SEQ ID NO: 9 - DNA, 264 bp, Synthetic construct
  source 1..264: mol_type = other DNA; note = Hepatocyte-myocyte bi-specific promoter HC6 (sH + truncated and modified human MCK) nucleotide sequence
ggcaaagtccatattgacttaggttaatcattaactctttgttgactaagtcaataatcgctgcccttgtaaggaggcgagatctagagacacccgagacgcccggttataattaaccaggacacgtggcgaacccccctccaacacctgccccctctaaaaataactcctttcccggcgggggccagctgtccccaccgccagggggcggggctataaaagccccaggctgggagcagccatcacagaagtccactcattctt

SEQ ID NO: 10 - DNA, 396 bp, Synthetic construct
  source 1..396: mol_type = other DNA; note = Hepatocyte-myocyte bi-specific promoter HC7 (sH + MCK-D3) nucleotide sequence
ggcaaagtccatattgacttaggttaatcattaactctttgttgactaagtcaataatcctcgagtgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgcgggcagcccatacaaggccatggggctgggcaagctgcacgccaggggcccctccctggggacagcccctcctggctagtcacaccctgtaggctcctctatataacccaggggcacaggggctgccctcattctaccaccacctccacagcacagacagacactcaggagccagccagc

SEQ ID NO: 11 - DNA, 386 bp, Synthetic construct
  source 1..386: mol_type = other DNA; note = Truncated mouse muscle creatine kinase (MCK) promoter MCK-D1 nucleotide sequence
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgcgggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 12 - DNA, 381 bp, Synthetic construct
  source 1..381: mol_type = other DNA; note = Truncated mouse muscle creatine kinase (MCK) promoter MCK-D2 nucleotide sequence
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgccaggggcccctccctggggacagcccctcctggctagtcacaccctgtaggctcctctatataacccaggggcacaggggctgccctcattctaccaccacctccacagcacagacagacactcaggagccagccagc

SEQ ID NO: 13 - DNA, 331 bp, Synthetic construct
  source 1..331: mol_type = other DNA; note = Truncated mouse muscle creatine kinase (MCK) promoter MCK-D3 nucleotide sequence
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgcgggcagcccatacaaggccatggggctgggcaagctgcacgccaggggcccctccctggggacagcccctcctggctagtcacaccctgtaggctcctctatataacccaggggcacaggggctgccctcattctaccaccacctccacagcacagacagacactcaggagccagccagc

SEQ ID NO: 14 - DNA, 436 bp, Synthetic construct
  source 1..436: mol_type = other DNA; note = CK8e promoter nucleotide sequence
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 15 - DNA, 144 bp, Synthetic construct
  source 1..144: mol_type = other DNA; note = Truncated and modified human muscle creatine kinase (MCK) promoter MCK-D4 nucleotide sequence
gctgcccttgtaaggaggcgagatctagagacacccgagacgcccggttataattaaccaggacacgtggcgaacccccctccaacacctgccccctctaaaaataactcctttcccggcgggggccagctgtccccaccgcca

SEQ ID NO: 16 - DNA, 1718 bp, Synthetic construct
  source 1..1718: mol_type = other DNA; note = Nucleotide sequence of human alpha-galactosidase A expression cassette containing promoter HC1, hGLAco1 and bovine growth hormone (bGH) poly A sequences (Cassette 1)
ggcaaagtccatattgacttaggttaatcattaactctttgttgactaagtcaataatcggggttatttttaaactatctgctcatctacttccggtctttccattcaggaatgcctttcttgggggggcggggctataaaagccccaggctgggagcagccatcacagaagtccactcattcttgcgatcgcctcgagagaattcgccaccatgcagctgaggaaccctgagctgcacctgggctgtgccctggccctgaggttcctggccctggtgagctgggacatccccggggctagagccctggacaatggcctggctaggacccccaccatgggctggctgcactgggagaggttcatgtgcaacctggactgccaagaggagcctgacagctgcatcagtgagaagctgttcatggagatggctgagctgatggtgtcagagggctggaaggatgccggctatgagtacctgtgcattgatgactgctggatggcccctcagagagactcagaaggcagactgcaagctgaccctcagaggttcccccatggcatcaggcagctggccaactatgtgcacagcaagggcctgaagctgggcatctatgcagatgtgggcaacaagacctgtgccggcttccccggcagctttggctactatgacattgatgcccagacctttgctgactggggagtggacctgctgaagtttgatggctgctactgtgacagcctggagaacctggcagatggctacaagcacatgagcctggccctgaacaggaccggcagaagcattgtgtacagctgtgagtggcccctgtacatgtggccctttcagaagcccaactacacagagatcagacagtactgcaaccactggaggaactttgctgacattgatgacagctggaagagcatcaagagcatcctggactggacaagcttcaaccaagagaggattgtggatgtggccggccccggcggctggaatgaccctgacatgctggtgattggcaactttggcctgagctggaatcagcaagtgacacagatggccctgtgggccatcatggctgcccccctgttcatgagcaatgacctgaggcacatcagcccccaagccaaggccctgctgcaagacaaggatgtgattgccatcaaccaagaccccctgggcaagcaaggctatcagctgaggcaaggagacaactttgaagtgtgggagagacccctgagcggcctggcctgggctgtggccatgatcaacaggcaagagattggcggccctagaagctacaccattgctgtggctagcctgggcaagggagtggcctgcaaccctgcctgcttcatcacacagctgctgcctgtgaagaggaagctgggcttctatgagtggacaagcaggctgagatcacacatcaaccccaccggcactgtgctgctgcagctggagaacaccatgcagatgagcctgaaggacctgctgtgagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 17 - DNA, 1797 bp, Synthetic construct
  source 1..1797: mol_type = other DNA; note = Nucleotide sequence of human alpha-galactosidase A expression cassette containing promoter HC6, hGLAco1 and bovine growth hormone (bGH) poly A sequences (Cassette 2)
ggcaaagtccatattgacttaggttaatcattaactctttgttgactaagtcaataatcgctgcccttgtaaggaggcgagatctagagacacccgagacgcccggttataattaaccaggacacgtggcgaacccccctccaacacctgccccctctaaaaataactcctttcccggcgggggccagctgtccccaccgccagggggcggggctataaaagccccaggctgggagcagccatcacagaagtccactcattcttgcgatcgcctcgagagaattcgccaccatgcagctgaggaaccctgagctgcacctgggctgtgccctggccctgaggttcctggccctggtgagctgggacatccccggggctagagccctggacaatggcctggctaggacccccaccatgggctggctgcactgggagaggttcatgtgcaacctggactgccaagaggagcctgacagctgcatcagtgagaagctgttcatggagatggctgagctgatggtgtcagagggctggaaggatgccggctatgagtacctgtgcattgatgactgctggatggcccctcagagagactcagaaggcagactgcaagctgaccctcagaggttcccccatggcatcaggcagctggccaactatgtgcacagcaagggcctgaagctgggcatctatgcagatgtgggcaacaagacctgtgccggcttccccggcagctttggctactatgacattgatgcccagacctttgctgactggggagtggacctgctgaagtttgatggctgctactgtgacagcctggagaacctggcagatggctacaagcacatgagcctggccctgaacaggaccggcagaagcattgtgtacagctgtgagtggcccctgtacatgtggccctttcagaagcccaactacacagagatcagacagtactgcaaccactggaggaactttgctgacattgatgacagctggaagagcatcaagagcatcctggactggacaagcttcaaccaagagaggattgtggatgtggccggccccggcggctggaatgaccctgacatgctggtgattggcaactttggcctgagctggaatcagcaagtgacacagatggccctgtgggccatcatggctgcccccctgttcatgagcaatgacctgaggcacatcagcccccaagccaaggccctgctgcaagacaaggatgtgattgccatcaaccaagaccccctgggcaagcaaggctatcagctgaggcaaggagacaactttgaagtgtgggagagacccctgagcggcctggcctgggctgtggccatgatcaacaggcaagagattggcggccctagaagctacaccattgctgtggctagcctgggcaagggagtggcctgcaaccctgcctgcttcatcacacagctgctgcctgtgaagaggaagctgggcttctatgagtggacaagcaggctgagatcacacatcaaccccaccggcactgtgctgctgcagctggagaacaccatgcagatgagcctgaaggacctgctgtgagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 18 - DNA, 1914 bp, Synthetic construct
  source 1..1914: mol_type = other DNA; note = Nucleotide sequence of human alpha-galactosidase A expression cassette containing promoter HC7, hGLAco1 and bovine growth hormone (bGH) poly A sequences (Cassette 3)
ggcaaagtccatattgacttaggttaatcattaactctttgttgactaagtcaataatcctcgagtgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgcgggcagcccatacaaggccatggggctgggcaagctgcacgccaggggcccctccctggggacagcccctcctggctagtcacaccctgtaggctcctctatataacccaggggcacaggggctgccctcattctaccaccacctccacagcacagacagacactcaggagccagccagcggatccgccaccatgcagctgaggaaccctgagctgcacctgggctgtgccctggccctgaggttcctggccctggtgagctgggacatccccggggctagagccctggacaatggcctggctaggacccccaccatgggctggctgcactgggagaggttcatgtgcaacctggactgccaagaggagcctgacagctgcatcagtgagaagctgttcatggagatggctgagctgatggtgtcagagggctggaaggatgccggctatgagtacctgtgcattgatgactgctggatggcccctcagagagactcagaaggcagactgcaagctgaccctcagaggttcccccatggcatcaggcagctggccaactatgtgcacagcaagggcctgaagctgggcatctatgcagatgtgggcaacaagacctgtgccggcttccccggcagctttggctactatgacattgatgcccagacctttgctgactggggagtggacctgctgaagtttgatggctgctactgtgacagcctggagaacctggcagatggctacaagcacatgagcctggccctgaacaggaccggcagaagcattgtgtacagctgtgagtggcccctgtacatgtggccctttcagaagcccaactacacagagatcagacagtactgcaaccactggaggaactttgctgacattgatgacagctggaagagcatcaagagcatcctggactggacaagcttcaaccaagagaggattgtggatgtggccggccccggcggctggaatgaccctgacatgctggtgattggcaactttggcctgagctggaatcagcaagtgacacagatggccctgtgggccatcatggctgcccccctgttcatgagcaatgacctgaggcacatcagcccccaagccaaggccctgctgcaagacaaggatgtgattgccatcaaccaagaccccctgggcaagcaaggctatcagctgaggcaaggagacaactttgaagtgtgggagagacccctgagcggcctggcctgggctgtggccatgatcaacaggcaagagattggcggccctagaagctacaccattgctgtggctagcctgggcaagggagtggcctgcaaccctgcctgcttcatcacacagctgctgcctgtgaagaggaagctgggcttctatgagtggacaagcaggctgagatcacacatcaaccccaccggcactgtgctgctgcagctggagaacaccatgcagatgagcctgaaggacctgctgtgagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 19 - DNA, 1908 bp, Synthetic construct
  source 1..1908: mol_type = other DNA; note = Nucleotide sequence of truncated human alpha-galactosidase A expression cassette containing promoter HC7, thGLAco1 and bovine growth hormone (bGH) poly A sequences (Cassette 4)
ggcaaagtccatattgacttaggttaatcattaactctttgttgactaagtcaataatcctcgagtgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgcgggcagcccatacaaggccatggggctgggcaagctgcacgccaggggcccctccctggggacagcccctcctggctagtcacaccctgtaggctcctctatataacccaggggcacaggggctgccctcattctaccaccacctccacagcacagacagacactcaggagccagccagcggatccgccaccatgcagctgaggaaccctgagctgcacctgggctgtgccctggccctgaggttcctggccctggtgagctgggacatccccggggctagagccctggacaatggcctggctaggacccccaccatgggctggctgcactgggagaggttcatgtgcaacctggactgccaagaggagcctgacagctgcatcagtgagaagctgttcatggagatggctgagctgatggtgtcagagggctggaaggatgccggctatgagtacctgtgcattgatgactgctggatggcccctcagagagactcagaaggcagactgcaagctgaccctcagaggttcccccatggcatcaggcagctggccaactatgtgcacagcaagggcctgaagctgggcatctatgcagatgtgggcaacaagacctgtgccggcttccccggcagctttggctactatgacattgatgcccagacctttgctgactggggagtggacctgctgaagtttgatggctgctactgtgacagcctggagaacctggcagatggctacaagcacatgagcctggccctgaacaggaccggcagaagcattgtgtacagctgtgagtggcccctgtacatgtggccctttcagaagcccaactacacagagatcagacagtactgcaaccactggaggaactttgctgacattgatgacagctggaagagcatcaagagcatcctggactggacaagcttcaaccaagagaggattgtggatgtggccggccccggcggctggaatgaccctgacatgctggtgattggcaactttggcctgagctggaatcagcaagtgacacagatggccctgtgggccatcatggctgcccccctgttcatgagcaatgacctgaggcacatcagcccccaagccaaggccctgctgcaagacaaggatgtgattgccatcaaccaagaccccctgggcaagcaaggctatcagctgaggcaaggagacaactttgaagtgtgggagagacccctgagcggcctggcctgggctgtggccatgatcaacaggcaagagattggcggccctagaagctacaccattgctgtggctagcctgggcaagggagtggcctgcaaccctgcctgcttcatcacacagctgctgcctgtgaagaggaagctgggcttctatgagtggacaagcaggctgagatcacacatcaaccccaccggcactgtgctgctgcagctggagaacaccatgcagatgagcctgaaggactgagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 20 - DNA, 1284 bp, Synthetic construct
  source 1..1284: mol_type = other DNA; note = Nucleotide sequence of codon-optimized human alpha-galactosidase A with 2 amino acids truncation at C-terminus (thGLAco1)
atgcagctgaggaaccctgagctgcacctgggctgtgccctggccctgaggttcctggccctggtgagctgggacatccccggggctagagccctggacaatggcctggctaggacccccaccatgggctggctgcactgggagaggttcatgtgcaacctggactgccaagaggagcctgacagctgcatcagtgagaagctgttcatggagatggctgagctgatggtgtcagagggctggaaggatgccggctatgagtacctgtgcattgatgactgctggatggcccctcagagagactcagaaggcagactgcaagctgaccctcagaggttcccccatggcatcaggcagctggccaactatgtgcacagcaagggcctgaagctgggcatctatgcagatgtgggcaacaagacctgtgccggcttccccggcagctttggctactatgacattgatgcccagacctttgctgactggggagtggacctgctgaagtttgatggctgctactgtgacagcctggagaacctggcagatggctacaagcacatgagcctggccctgaacaggaccggcagaagcattgtgtacagctgtgagtggcccctgtacatgtggccctttcagaagcccaactacacagagatcagacagtactgcaaccactggaggaactttgctgacattgatgacagctggaagagcatcaagagcatcctggactggacaagcttcaaccaagagaggattgtggatgtggccggccccggcggctggaatgaccctgacatgctggtgattggcaactttggcctgagctggaatcagcaagtgacacagatggccctgtgggccatcatggctgcccccctgttcatgagcaatgacctgaggcacatcagcccccaagccaaggccctgctgcaagacaaggatgtgattgccatcaaccaagaccccctgggcaagcaaggctatcagctgaggcaaggagacaactttgaagtgtgggagagacccctgagcggcctggcctgggctgtggccatgatcaacaggcaagagattggcggccctagaagctacaccattgctgtggctagcctgggcaagggagtggcctgcaaccctgcctgcttcatcacacagctgctgcctgtgaagaggaagctgggcttctatgagtggacaagcaggctgagatcacacatcaaccccaccggcactgtgctgctgcagctggagaacaccatgcagatgagcctgaaggactga

SEQ ID NO: 21 - protein, 736 aa, Adeno-associated virus
  source 1..736: note = wild-type AAV9 VP1 amino acid sequence (position 586-588)(Uniprot Q6JC40_9VIRU) VP1: amino acids 1-736; VP2: amino acids 138-736; VP3: amino acids 203-736
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - protein, 599 aa, Adeno-associated virus
  source 1..599: note = wild-type AAV9 VP2 amino acid sequence
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 534 aa, Adeno-associated virus
  source 1..534: note = wild-type AAV9 VP3 amino acid sequence
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 743 aa, Synthetic construct
  source 1..743: note = AVT901 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGHMNGRNNSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 7 aa, Synthetic construct
  source 1..7: note = AVT901 VP1s insert peptide amino acid sequence
HMNGRNN

SEQ ID NO: 26 - protein, 743 aa, Synthetic construct
  source 1..743: note = AVT902 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGPHYTHGISGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 27 - protein, 7 aa, Synthetic construct
  source 1..7: note = AVT902 VP1s insert peptide amino acid sequence
PHYTHGI

SEQ ID NO: 28 - protein, 745 aa, Synthetic construct
  source 1..745: note = AVT903 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDTNNTNMAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 9 aa, Synthetic construct
  source 1..9: note = AVT903 VP1s insert peptide amino acid sequence
RGDTNNTNM

SEQ ID NO: 30 - protein, 743 aa, Synthetic construct
  source 1..743: note = AVT905 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSRGDRPMAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - protein, 7 aa, Synthetic construct
  source 1..7: note = AVT905 VP1s insert peptide amino acid sequence
SRGDRPM

SEQ ID NO: 32 - protein, 743 aa, Synthetic construct
  source 1..743: note = AVT906 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLHKGIDIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 7 aa, Synthetic construct
  source 1..7: note = AVT906 VP1s insert peptide amino acid sequence
LHKGIDI

SEQ ID NO: 34 - protein, 750 aa, Synthetic construct
  source 1..750: note = AVT907 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGHMNGRNNSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGPGRGDQTTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - protein, 7 aa, Synthetic construct
  source 1..7: note = AVT907 VP1s insert peptide (after wild-type position 453) amino acid sequence
HMNGRNN

SEQ ID NO: 36 - protein, 10 aa, Synthetic construct
  source 1..10: note = AVT907 VP1s amino acid sequence corresponding to position 586-588 of wild-type AAV9 VP1, plus insert peptide (after wild-type position 588)
GPGRGDQTTL

SEQ ID NO: 37 - protein, 743 aa, Synthetic construct; identical to SEQ ID NO: 30
  source 1..743: note = AVT908 VP1 amino acid sequence

SEQ ID NO: 38 - protein, 7 aa, Synthetic construct
  source 1..7: note = AVT908 VP1s insert peptide amino acid sequence
SRGDRPM

SEQ ID NO: 39 - protein, 743 aa, Synthetic construct
  source 1..743: note = AVT909 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGVNGRSTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 7 aa, Synthetic construct
  source 1..7: note = AVT909 VP1s insert peptide amino acid sequence
GVNGRST

SEQ ID NO: 41 - protein, 743 aa, Synthetic construct
  source 1..743: note = AVT910 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGVNGRQVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 42 - protein, 7 aa, Synthetic construct
  source 1..7: note = AVT910 VP1s insert peptide amino acid sequence
GVNGRQV

SEQ ID NO: 43 - protein, 743 aa, Synthetic construct
  source 1..743: note = AVT911 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGQTYTHNKSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 44 - protein, 7 aa, Synthetic construct
  source 1..7: note = AVT911 VP1s insert peptide amino acid sequence
QTYTHNK

SEQ ID NO: 45 - protein, 750 aa, Synthetic construct
  source 1..750: note = AVT912 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGQTYTHNKSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSRGDRPMAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 7 aa, Synthetic construct
  source 1..7: note = AVT912 VP1s insert peptide (after wild-type position 453) amino acid sequence
QTYTHNK

SEQ ID NO: 47 - protein, 7 aa, Synthetic construct
  source 1..7: note = AVT912 VP1s insert peptide (after wild-type position 588) amino acid sequence
SRGDRPM

SEQ ID NO: 48 - protein, 743 aa, Synthetic construct
  source 1..743: note = AVT913 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDTNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 49 - protein, 10 aa, Synthetic construct
  source 1..10: note = AVT913 VP1s amino acid sequence corresponding to position 586-588 of wild-type AAV9 VP1 plus insert peptide
ENRRGDTNAL

SEQ ID NO: 50 - protein, 743 aa, Synthetic construct
  source 1..743: note = AVT914 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNSRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 10 aa, Synthetic construct
  source 1..10: note = AVT914 VP1s amino acid sequence corresponding to position 586-588 of wild-type AAV9 VP1 plus insert peptide
NNSRGDHNSL

SEQ ID NO: 52 - protein, 743 aa, Synthetic construct
  source 1..743: note = AVT915 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNSRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 53 - protein, 10 aa, Synthetic construct
  source 1..10: note = AVT915 VP1s amino acid sequence corresponding to position 586-588 of wild-type AAV9 VP1 plus insert peptide
NNSRGDYNSL

SEQ ID NO: 54 - protein, 743 aa, Synthetic construct
  source 1..743: note = AVT916 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNLRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 55 - protein, 10 aa, Synthetic construct
  source 1..10: note = AVT916 VP1s amino acid sequence corresponding to position 586-588 of wild-type AAV9 VP1 plus insert peptide
NNLRGDHNSL

SEQ ID NO: 56 - protein, 743 aa, Synthetic construct
  source 1..743: note = AVT917 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNARGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 57 - protein, 10 aa, Synthetic construct
  source 1..10: note = AVT917 VP1s amino acid sequence corresponding to position 586-588 of wild-type AAV9 VP1 plus insert peptide
NNARGDHNSL

SEQ ID NO: 58 - protein, 743 aa, Synthetic construct
  source 1..743: note = AVT918 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGNIRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 59 - protein, 10 aa, Synthetic construct
  source 1..10: note = AVT918 VP1s amino acid sequence corresponding to position 586-588 of wild-type AAV9 VP1 plus insert peptide
GNIRGDHNTL

SEQ ID NO: 60 - protein, 743 aa, Synthetic construct
  source 1..743: note = AVT919 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMNVRGDTNKLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 61 - protein, 10 aa, Synthetic construct
  source 1..10: note = AVT919 VP1s amino acid sequence corresponding to position 586-588 of wild-type AAV9 VP1 plus insert peptide
MNVRGDTNKL

SEQ ID NO: 62 - protein, 743 aa, Synthetic construct
  source 1..743: note = AVT920 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTNMRGDHNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 63 - protein, 10 aa, Synthetic construct
  source 1..10: note = AVT920 VP1s amino acid sequence corresponding to position 586-588 of wild-type AAV9 VP1 plus insert peptide
TNMRGDHNAL

SEQ ID NO: 64 - protein, 743 aa, Synthetic construct
  source 1..743: note = AV9ML001 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNNRGDNNYLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 65 - protein, 10 aa, Synthetic construct
  source 1..10: note = AV9ML001 VP1s amino acid sequence corresponding to position 586-588 of wild-type AAV9 VP1 plus insert peptide
NNNRGDNNYL

SEQ ID NO: 66 - protein, 743 aa, Synthetic construct
  source 1..743: note = AV9ML002 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNNRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 67 - protein, 10 aa, Synthetic construct
  source 1..10: note = AV9ML002 VP1s amino acid sequence corresponding to position 586-588 of wild-type AAV9 VP1 plus insert peptide
NNNRGDHNTL

SEQ ID NO: 68 - protein, 743 aa, Synthetic construct
  source 1..743: note = AV9ML004 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNERGDRNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 69 - protein, 10 aa, Synthetic construct
  source 1..10: note = AV9ML004 VP1s amino acid sequence corresponding to position 586-588 of wild-type AAV9 VP1 plus insert peptide
NNERGDRNSL

SEQ ID NO: 70 - protein, 743 aa, Synthetic construct
  source 1..743: note = AV9ML005 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNSRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 71 - protein, 10 aa, Synthetic construct
  source 1..10: note = AV9ML005 VP1s amino acid sequence corresponding to position 586-588 of wild-type AAV9 VP1 plus insert peptide
NNSRGDHNTL

SEQ ID NO: 72 - protein, 743 aa, Synthetic construct
  source 1..743: note = AV9ML007 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENKRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 73 - protein, 10 aa, Synthetic construct
  source 1..10: note = AV9ML007 VP1s amino acid sequence corresponding to position 586-588 of wild-type AAV9 VP1 plus insert peptide
ENKRGDHNSL

SEQ ID NO: 74 - protein, 743 aa, Synthetic construct
  source 1..743: note = AV9ML012 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQINSRGDHNVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 75 - protein, 10 aa, Synthetic construct
  source 1..10: note = AV9ML012 VP1s amino acid sequence corresponding to position 586-588 of wild-type AAV9 VP1 plus insert peptide
INSRGDHNVL

SEQ ID NO: 76 - protein, 743 aa, Synthetic construct
  source 1..743: note = AV9ML014 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNNRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 77 - protein, 10 aa, Synthetic construct
  source 1..10: note = AV9ML014 VP1s amino acid sequence corresponding to position 586-588 of wild-type AAV9 VP1 plus insert peptide
NNNRGDYNSL

SEQ ID NO: 78 - protein, 743 aa, Synthetic construct
  source 1..743: note = AV9ML016 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDKLSIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 79 - protein, 7 aa, Synthetic construct
  source 1..7: note = AV9ML016 VP1s insert peptide amino acid sequence
RGDKLSI

SEQ ID NO: 80 - protein, 745 aa, Synthetic construct
  source 1..745: note = AV9ML018 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLNTNNSFTKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 81 - protein, 9 aa, Synthetic construct
  source 1..9: note = AV9ML018 VP1s insert peptide amino acid sequence
LNTNNSFTK

SEQ ID NO: 82 - protein, 743 aa, Synthetic construct
  source 1..743: note = AV9ML025 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNSRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 83 - protein, 10 aa, Synthetic construct
  source 1..10: note = AV9ML025 VP1s amino acid sequence corresponding to position 586-588 of wild-type AAV9 VP1 plus insert peptide insert peptide
SNSRGDHNTL

SEQ ID NO: 84 - protein, 743 aa, Synthetic construct
  source 1..743: note = AV9ML026 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNSRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 85 - protein, 10 aa, Synthetic construct
  source 1..10: note = AV9ML026 VP1s amino acid sequence corresponding to position 586-588 of wild-type AAV9 VP1 plus insert peptide insert peptide
SNSRGDHNSL

SEQ ID NO: 86 - protein, 743 aa, Synthetic construct
  source 1..743: note = AV9ML027 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 87 - protein, 10 aa, Synthetic construct
  source 1..10: note = AV9ML027 VP1s amino acid sequence corresponding to position 586-588 of wild-type AAV9 VP1 plus insert peptide insert peptide
ENRRGDHNSL

SEQ ID NO: 88 - protein, 743 aa, Synthetic construct
  source 1..743: note = AV9ML028 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGNSRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 89 - protein, 10 aa, Synthetic construct
  source 1..10: note = AV9ML028 VP1s amino acid sequence corresponding to position 586-588 of wild-type AAV9 VP1 plus insert peptide insert peptide
GNSRGDYNSL

SEQ ID NO: 90 - protein, 743 aa, Synthetic construct
  source 1..743: note = AV9ML029 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNSRGDRNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 91 - protein, 10 aa, Synthetic construct
  source 1..10: note = AV9ML029 VP1s amino acid sequence corresponding to position 586-588 of wild-type AAV9 VP1 plus insert peptide insert peptide
NNSRGDRNSL

SEQ ID NO: 92 - protein, 743 aa, Synthetic construct
  source 1..743: note = AV9ML030 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMNVRGDTNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 93 - protein, 10 aa, Synthetic construct
  source 1..10: note = AV9ML030 VP1s amino acid sequence corresponding to position 586-588 of wild-type AAV9 VP1 plus insert peptide insert peptide
MNVRGDTNSL

SEQ ID NO: 94 - protein, 743 aa, Synthetic construct
  source 1..743: note = AV9ML031 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNARGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 95 - protein, 10 aa, Synthetic construct
  source 1..10: note = AV9ML031 VP1s amino acid sequence corresponding to position 586-588 of wild-type AAV9 VP1 plus insert peptide insert peptide
SNARGDHNSL

SEQ ID NO: 96 - protein, 743 aa, Synthetic construct
  source 1..743: note = AV9ML032 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGNSRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 97 - protein, 10 aa, Synthetic construct
  source 1..10: note = AV9ML032 VP1s amino acid sequence corresponding to position 586-588 of wild-type AAV9 VP1 plus insert peptide insert peptide
GNSRGDHNTL

SEQ ID NO: 98 - protein, 743 aa, Synthetic construct
  source 1..743: note = AV9ML033 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNNRGDHNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 99 - protein, 10 aa, Synthetic construct
  source 1..10: note = AV9ML033 VP1s amino acid sequence corresponding to position 586-588 of wild-type AAV9 VP1 plus insert peptide insert peptide
NNNRGDHNAL

SEQ ID NO: 100 - protein, 743 aa, Synthetic construct
  source 1..743: note = AV9ML034 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNLRGDRNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 101 - protein, 10 aa, Synthetic construct
  source 1..10: note = AV9ML034 VP1s amino acid sequence corresponding to position 586-588 of wild-type AAV9 VP1 plus insert peptide insert peptide
NNLRGDRNSL

SEQ ID NO: 102 - protein, 743 aa, Synthetic construct
  source 1..743: note = AV9ML035 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDNRRGDTNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 103 - protein, 10 aa, Synthetic construct
  source 1..10: note = AV9ML035 VP1s amino acid sequence corresponding to position 586-588 of wild-type AAV9 VP1 plus insert peptide insert peptide
DNRRGDTNAL

SEQ ID NO: 104 - protein, 743 aa, Synthetic construct
  source 1..743: note = AV9ML036 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENERGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 105 - protein, 10 aa, Synthetic construct
  source 1..10: note = AV9ML036 VP1s amino acid sequence corresponding to position 586-588 of wild-type AAV9 VP1 plus insert peptide insert peptide
ENERGDHNSL

SEQ ID NO: 106 - protein, 743 aa, Synthetic construct
  source 1..743: note = AV9ML037 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNARGDTNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 107 - protein, 10 aa, Synthetic construct
  source 1..10: note = AV9ML037 VP1s amino acid sequence corresponding to position 586-588 of wild-type AAV9 VP1 plus insert peptide insert peptide
NNARGDTNAL

SEQ ID NO: 108 - protein, 743 aa, Synthetic construct
  source 1..743: note = AV9ML038 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNLRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 109 - protein, 10 aa, Synthetic construct
  source 1..10: note = AV9ML038 VP1s amino acid sequence corresponding to position 586-588 of wild-type AAV9 VP1 plus insert peptide insert peptide
SNLRGDHNSL

SEQ ID NO: 110 - protein, 743 aa, Synthetic construct
  source 1..743: note = AV9ML039 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMSVRGDTNKLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 111 - protein, 10 aa, Synthetic construct
  source 1..10: note = AV9ML039 VP1s amino acid sequence corresponding to position 586-588 of wild-type AAV9 VP1 plus insert peptide insert peptide
MSVRGDTNKL

SEQ ID NO: 112 - protein, 743 aa, Synthetic construct
  source 1..743: note = AV9ML040 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMNVRGDTNKPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 113 - protein, 10 aa, Synthetic construct
  source 1..10: note = AV9ML040 VP1s amino acid sequence corresponding to position 586-588 of wild-type AAV9 VP1 plus insert peptide insert peptide
MNVRGDTNKP

SEQ ID NO: 114 - protein, 743 aa, Synthetic construct
  source 1..743: note = AV9ML041 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNLRGDTNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 115 - protein, 10 aa, Synthetic construct
  source 1..10: note = AV9ML041 VP1s amino acid sequence corresponding to position 586-588 of wild-type AAV9 VP1 plus insert peptide insert peptide
NNLRGDTNAL

SEQ ID NO: 116 - protein, 743 aa, Synthetic construct
  source 1..743: note = AV9ML042 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQINSRGDYNVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 117 - protein, 10 aa, Synthetic construct
  source 1..10: note = AV9ML042 VP1s amino acid sequence corresponding to position 586-588 of wild-type AAV9 VP1 plus insert peptide insert peptide
INSRGDYNVL

SEQ ID NO: 118 - protein, 743 aa, Synthetic construct
  source 1..743: note = AV9ML043 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQKNRRGDTNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 119 - protein, 10 aa, Synthetic construct
  source 1..10: note = AV9ML043 VP1s amino acid sequence corresponding to position 586-588 of wild-type AAV9 VP1 plus insert peptide insert peptide
KNRRGDTNAL

SEQ ID NO: 120 - protein, 743 aa, Synthetic construct
  source 1..743: note = AV9ML044 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENERGDRNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 121 - protein, 10 aa, Synthetic construct
  source 1..10: note = AV9ML044 VP1s amino acid sequence corresponding to position 586-588 of wild-type AAV9 VP1 plus insert peptide insert peptide
ENERGDRNSL

SEQ ID NO: 122 - protein, 743 aa, Synthetic construct
  source 1..743: note = AV9ML045 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMNARGDTNKLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 123 - protein, 10 aa, Synthetic construct
  source 1..10: note = AV9ML045 VP1s amino acid sequence corresponding to position 586-588 of wild-type AAV9 VP1 plus insert peptide insert peptide
MNARGDTNKL

SEQ ID NO: 124 - protein, 743 aa, Synthetic construct
  source 1..743: note = AV9ML046 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGNIRGDRNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 125 - protein, 10 aa, Synthetic construct
  source 1..10: note = AV9ML046 VP1s amino acid sequence corresponding to position 586-588 of wild-type AAV9 VP1 plus insert peptide insert peptide
GNIRGDRNTL

SEQ ID NO: 126 - protein, 743 aa, Synthetic construct
  source 1..743: note = AV9ML047 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGNIRGDHNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 127 - protein, 10 aa, Synthetic construct
  source 1..10: note = AV9ML047 VP1s amino acid sequence corresponding to position 586-588 of wild-type AAV9 VP1 plus insert peptide insert peptide
GNIRGDHNAL

SEQ ID NO: 128 - protein, 743 aa, Synthetic construct
  source 1..743: note = AV9ML048 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNRRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 129 - protein, 10 aa, Synthetic construct
  source 1..10: note = AV9ML048 VP1s amino acid sequence corresponding to position 586-588 of wild-type AAV9 VP1 plus insert peptide insert peptide
NNRRGDHNSL

SEQ ID NO: 130 - protein, 743 aa, Synthetic construct
  source 1..743: note = AV9ML049 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTNIRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 131 - protein, 10 aa, Synthetic construct
  source 1..10: note = AV9ML049 VP1s amino acid sequence corresponding to position 586-588 of wild-type AAV9 VP1 plus insert peptide insert peptide
TNIRGDYNSL

SEQ ID NO: 132 - protein, 743 aa, Synthetic construct
  source 1..743: note = AV9ML050 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQRNLRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 133 - protein, 10 aa, Synthetic construct
  source 1..10: note = AV9ML050 VP1s amino acid sequence corresponding to position 586-588 of wild-type AAV9 VP1 plus insert peptide insert peptide
RNLRGDHNTL

SEQ ID NO: 134 - protein, 743 aa, Synthetic construct
  source 1..743: note = AV9ML051 VP1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQCNSRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 135 - protein, 10 aa, Synthetic construct
  source 1..10: note = AV9ML051 VP1s amino acid sequence corresponding to position 586-588 of wild-type AAV9 VP1 plus insert peptide insert peptide
CNSRGDHNTL

SEQ ID NO: 136 - protein, 735 aa, Adeno-associated virus [sequence omitted]

SEQ ID NO: 137 - protein, 738 aa, Adeno-associated virus
  source 1..738: note = AAV8 (Uniprot Q8JQF8_9VIRU) VP1: amino acids 1-738; VP2: amino acids 138-738; VP3: amino acids 204-738
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 138 - protein, 736 aa, Adeno-associated virus
  source 1..736: note = AAVrh8 (Uniprot Q808Y3_9VIRU) VP1: amino acids 1-736; VP2: amino acids 138-736; VP3: amino acids 203-736
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 139 - protein, 738 aa, Adeno-associated virus
  source 1..738: note = AAVrh10 (Uniprot Q808W5_9VIRU) VP1: amino acids 1-738; VP2: amino acids 138-738; VP3: amino acids 204-738
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 140 - protein, 735 aa, Adeno-associated virus
  source 1..735: note = AAV2v; VP1: amino acids 1-735; VP2: amino acids 138-735; VP3: amino acids 204-735
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQGVSKVSADNNNSEFSWTGATKYHLNGRDSLVNPGPAMASHKDDEEKFFPQSGVLIFGKQGSEKTNVDIEKVMITDEEEIRTTNPVATEQYGSVSTNLQSGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 141 - protein, 736 aa, Adeno-associated virus
  source 1..736: note = AAV44-9; VP1: amino acids 1-736; VP2: amino acids 138-736; VP3: amino acids 204-736
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSNMASQARNWVPGPSYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

(19) United States
(12) Patent Application Publication
(10) Pub. No.: US 2026/0286389 A1
(43) Pub. Date: Sep. 24, 2026

(54) COMPOSITIONS AND METHODS FOR GENE THERAPIES

(71) Applicant: EXEGENESIS BIO CO., Lower Gwynedd, PA (US)

(72) Inventors: Shaoyong LI, Hopkinton, MA (US); Guojie YE, Gainesville, MA (US); Zhenhua WU, Lower Gwynedd, PA (US); Lijun WANG, Brisbane, CA (US)

(21) Appl. No.: 19/476,139

(22) PCT Filed: Apr. 16, 2024
(86) PCT No.: PCT/CN2024/088121

§ 371 Date: Oct. 16, 2025

(30) Foreign Application Priority Data
Apr. 17, 2023 (WO) PCT/CN2023/088738

(51) Int. Cl. C12N 15/86; A61K 35/76; A61K 38/00; A61K 48/00; A61P 3/00; C07K 14/005; C12N 9/40

(52) U.S. Cl. CPC C12N 15/86; A61K 35/76; A61K 48/0058; A61P 3/00; C07K 14/005; C12N 9/2465; A61K 38/00; C12N 2750/14122; C12N 2750/14143; C12N 2750/14145; C12N 2800/22; C12N 2830/008; C12Y 302/01022

(57) ABSTRACT

Provided herein are polynucleotides comprising a nucleotide sequence encoding a biologic molecule (such as human α-galactosidase A), a nucleotide sequence encoding a promoter (such as a hepatocyte-specific promoter or a hepatocyte-myocyte bispecific promoter), or a nucleotide sequence encoding an expression cassette (such as a human α-galactosidase A expression cassette). Also provided herein are promoters, expression cassettes, vectors, host cells, gene delivery systems (e.g., recombinant viral particles such as recombinant adeno-associated virus (AAV) particles and non-viral gene delivery systems), associated pharmaceutical compositions, as well as methods using the same. Such compositions and methods are particularly useful for gene therapies, especially for lysosomal storage disorders including Fabry Disease.

### CROSS REFERENCE TO RELATED APPLICATIONS

This application claims the benefit of, and priority to, PCT Application No. PCT/CN2023/088738, filed Apr. 17, 2023. The contents of these applications are incorporated herein by reference in their entireties for all purposes.

### TECHNICAL FIELD

Provided herein are polynucleotides comprising a nucleotide sequence encoding a biologic molecule (such as human α-galactosidase A), a nucleotide sequence encoding a promoter (such as a hepatocyte-specific promoter or a hepatocyte-myocyte bispecific promoter), or a nucleotide sequence encoding an expression cassette (such as a human α-galactosidase A expression cassette). Also provided herein are promoters, expression cassettes, vectors, host cells, gene delivery systems (e.g., recombinant viral particles such as recombinant adeno-associated virus (AAV) particles and non-viral gene delivery systems), associated pharmaceutical compositions, as well as methods using the same. Such compositions and methods are particularly useful for gene therapies, especially for lysosomal storage disorders including Fabry Disease.

### BACKGROUND

Gene therapies including AAV-based gene therapies have the potential to become promising treatments for many diseases.

Lysosomal storage disorders are inherited metabolic disorders characterized by the accumulation of an abnormal amount of substrates in the cells of various organs resulting from the defective functioning of lysosomes. For example, Fabry disease (FD) is a rare λ-linked metabolic disorder caused by deficiency of lysosomal enzyme α-galactosidase A (α-Gal A) due to pathogenic mutations in the GLA gene. The progressive accumulation of substrates within lysosomes results in cellular dysfunction and multiple organ damage.

To date, there remains a need for developing compositions, systems and methods for gene therapies, in particular gene therapies for lysosomal storage disorders including Fabry disease.

### SUMMARY

In one aspect, provided herein is a polynucleotide comprising a nucleotide sequence encoding human α-galactosidase A, wherein the nucleotide sequence encoding human α-galactosidase A comprises or is set forth in SEQ ID NO:1. In certain embodiments, the nucleotide sequence encoding human α-galactosidase A comprises SEQ ID NO:1. In certain embodiments, the nucleotide sequence encoding human α-galactosidase A is set forth in SEQ ID NO:1.

In one aspect, provided herein is a polynucleotide comprising a nucleotide sequence encoding a promoter, wherein the nucleotide sequence encoding the promoter (i) comprises SEQ ID NO:3, SEQ ID NO:4, or SEQ ID NO:10, or (ii) is set forth in SEQ ID NO:3, SEQ ID NO:4, or SEQ ID NO:10. In specific embodiments, the promoter is a hepatocyte-specific promoter. In specific embodiments, the promoter is a hepatocyte-myocyte bispecific promoter. In certain embodiments, the nucleotide sequence encoding the promoter comprises SEQ ID NO:3. In certain embodiments, the nucleotide sequence encoding the promoter is set forth in SEQ ID NO:3. In certain embodiments, the nucleotide sequence encoding the promoter comprises SEQ ID NO:4. In certain embodiments, the nucleotide sequence encoding the promoter is set forth in SEQ ID NO:4. In certain embodiments, the nucleotide sequence encoding the promoter comprises SEQ ID NO:10. In certain embodiments, the nucleotide sequence encoding the promoter is set forth in SEQ ID NO:10.

In one aspect, provided herein is a polynucleotide comprising a nucleotide sequence encoding human α-galactosidase A and a nucleotide sequence encoding a promoter that is operably linked to the nucleotide sequence encoding human α-galactosidase A, wherein the nucleotide sequence encoding human α-galactosidase A comprises or is set forth in SEQ ID NO:1. In certain embodiments, the nucleotide sequence encoding human α-galactosidase A comprises SEQ ID NO:1. In certain embodiments, the nucleotide sequence encoding human α-galactosidase A is set forth in SEQ ID NO:1. In specific embodiments, the promoter is a hepatocyte-specific promoter. In specific embodiments, the promoter is a hepatocyte-myocyte bispecific promoter. In certain embodiments, the nucleotide sequence encoding the promoter comprises SEQ ID NO:3. In certain embodiments, the nucleotide sequence encoding the promoter is set forth in SEQ ID NO:3. In certain embodiments, the nucleotide sequence encoding the promoter comprises SEQ ID NO:4. In certain embodiments, the nucleotide sequence encoding the promoter is set forth in SEQ ID NO:4. In certain embodiments, the nucleotide sequence encoding the promoter comprises SEQ ID NO:10. In certain embodiments, the nucleotide sequence encoding the promoter is set forth in SEQ ID NO:10. In various embodiments, the polynucleotide further comprises a nucleotide sequence encoding a polyA signal.

In one aspect, provided herein is a polynucleotide comprising a nucleotide sequence encoding a biologic molecule and a nucleotide sequence encoding a promoter that is operably linked to the nucleotide sequence encoding the biologic molecule, wherein the nucleotide sequence encoding the promoter (i) comprises SEQ ID NO:3, SEQ ID NO:4, or SEQ ID NO:10, or (ii) is set forth in SEQ ID NO:3, SEQ ID NO:4, or SEQ ID NO:10. In specific embodiments, the promoter is a hepatocyte-specific promoter. In specific embodiments, the promoter is a hepatocyte-myocyte bispecific promoter. In certain embodiments, the nucleotide sequence encoding the promoter comprises SEQ ID NO:3. In certain embodiments, the nucleotide sequence encoding the promoter is set forth in SEQ ID NO:3. In certain embodiments, the nucleotide sequence encoding the promoter comprises SEQ ID NO:4. In certain embodiments, the nucleotide sequence encoding the promoter is set forth in SEQ ID NO:4. In certain embodiments, the nucleotide sequence encoding the promoter comprises SEQ ID NO:10. In certain embodiments, the nucleotide sequence encoding the promoter is set forth in SEQ ID NO:10. In specific embodiments, the biologic molecule is human α-galactosidase A. In various embodiments, the polynucleotide further comprises a nucleotide sequence encoding a polyA signal.

In one aspect, provided herein is a polynucleotide comprising a nucleotide sequence encoding an expression cassette, wherein the nucleotide sequence encoding the expression cassette comprises or is set forth in SEQ ID NO:16 or SEQ ID NO:18. In certain embodiments, the nucleotide sequence encoding the expression cassette comprises SEQ ID NO:16. In certain embodiments, the nucleotide sequence encoding the expression cassette is set forth in SEQ ID NO:16. In certain embodiments, the nucleotide sequence encoding the expression cassette comprises SEQ ID NO: 18. In certain embodiments, the nucleotide sequence encoding the expression cassette is set forth in SEQ ID NO:18.

In one aspect, provided herein is a promoter, which is encoded by a nucleotide sequence that (i) comprises SEQ ID NO:3, SEQ ID NO:4, or SEQ ID NO:10, or (ii) is set forth in SEQ ID NO:3, SEQ ID NO:4, or SEQ ID NO:10. In specific embodiments, the promoter is a hepatocyte-specific promoter. In specific embodiments, the promoter is a hepatocyte-myocyte bispecific promoter. In certain embodiments, the nucleotide sequence encoding the promoter comprises SEQ ID NO:3. In certain embodiments, the nucleotide sequence encoding the promoter is set forth in SEQ ID NO:3. In certain embodiments, the nucleotide sequence encoding the promoter comprises SEQ ID NO:4. In certain embodiments, the nucleotide sequence encoding the promoter is set forth in SEQ ID NO:4. In certain embodiments, the nucleotide sequence encoding the promoter comprises SEQ ID NO:10. In certain embodiments, the nucleotide sequence encoding the promoter is set forth in SEQ ID NO: 10.

In one aspect, provided herein is an expression cassette comprising a nucleotide sequence encoding human α-galactosidase A and a nucleotide sequence encoding a promoter that is operably linked to the nucleotide sequence encoding human α-galactosidase A, wherein the nucleotide sequence encoding human α-galactosidase A comprises or is set forth in SEQ ID NO:1. In certain embodiments, the nucleotide sequence encoding human α-galactosidase A comprises SEQ ID NO:1. In certain embodiments, the nucleotide sequence encoding human α-galactosidase A is set forth in SEQ ID NO:1. In specific embodiments, the promoter is a hepatocyte-specific promoter. In specific embodiments, the promoter is a hepatocyte-myocyte bispecific promoter. In certain embodiments, the nucleotide sequence encoding the promoter comprises SEQ ID NO:3. In certain embodiments, the nucleotide sequence encoding the promoter is set forth in SEQ ID NO:3. In certain embodiments, the nucleotide sequence encoding the promoter comprises SEQ ID NO:4. In certain embodiments, the nucleotide sequence encoding the promoter is set forth in SEQ ID NO:4. In certain embodiments, the nucleotide sequence encoding the promoter comprises SEQ ID NO:10. In certain embodiments, the nucleotide sequence encoding the promoter is set forth in SEQ ID NO:10. In various embodiments, the expression cassette further comprises a nucleotide sequence encoding a polyA signal.

In one aspect, provided herein is an expression cassette comprising a nucleotide sequence encoding a biologic molecule and a nucleotide sequence encoding a promoter that is operably linked to the nucleotide sequence encoding the biologic molecule, wherein the nucleotide sequence encoding the promoter (i) comprises SEQ ID NO:3, SEQ ID NO:4, or SEQ ID NO:10, or (ii) is set forth in SEQ ID NO:3, SEQ ID NO:4, or SEQ ID NO:10. In specific embodiments, the promoter is a hepatocyte-specific promoter. In specific embodiments, the promoter is a hepatocyte-myocyte bispecific promoter. In certain embodiments, the nucleotide sequence encoding the promoter comprises SEQ ID NO:3. In certain embodiments, the nucleotide sequence encoding the promoter is set forth in SEQ ID NO:3. In certain embodiments, the nucleotide sequence encoding the promoter comprises SEQ ID NO:4. In certain embodiments, the nucleotide sequence encoding the promoter is set forth in SEQ ID NO:4. In certain embodiments, the nucleotide sequence encoding the promoter comprises SEQ ID NO: 10. In certain embodiments, the nucleotide sequence encoding the promoter is set forth in SEQ ID NO:10. In specific embodiments, the biologic molecule is human α-galactosidase A. In various embodiments, the expression cassette further comprises a nucleotide sequence encoding a polyA signal.

In one aspect, provided herein is an expression cassette, which is encoded by a nucleotide sequence that (i) comprises SEQ ID NO:16 or SEQ ID NO:18, or (ii) is set forth in SEQ ID NO:16 or SEQ ID NO:18. In certain embodiments, the nucleotide sequence encoding the expression cassette comprises SEQ ID NO:16. In certain embodiments, the nucleotide sequence encoding the expression cassette is set forth in SEQ ID NO:16. In certain embodiments, the nucleotide sequence encoding the expression cassette comprises SEQ ID NO:18. In certain embodiments, the nucleotide sequence encoding the expression cassette is set forth in SEQ ID NO:18.

In one aspect, provided herein is a polynucleotide comprising a nucleotide sequence encoding an expression cassette described herein.

In one aspect, provided herein is a vector comprising a polynucleotide described herein, a promoter described herein, or an expression cassette described herein.

In one aspect, provided herein is a recombinant viral particle comprising a recombinant viral genome comprising an expression cassette described herein.

In one aspect, provided herein is a recombinant adeno-associated virus (AAV) particle comprising: (a) an AAV capsid; and (b) a recombinant AAV genome comprising an expression cassette described herein flanked by AAV-inverted terminal repeats (ITRs). In specific embodiments, the recombinant AAV particle is a recombinant AAV serotype 9 (rAAV9) particle. In specific embodiments, the AAV capsid comprises a variant AAV9 capsid protein. In certain embodiments, the variant AAV9 capsid protein comprises the amino acid sequence of SEQ ID NO:31, 38, 49, 51, 53, 55, 57, 59 or 61. In certain embodiments, the variant AAV9 capsid protein comprises the amino acid sequence of SEQ ID NO:30, 37, 48, 50, 52, 54, 56, 58 or 60.

In one aspect, provided herein is a host cell comprising a polynucleotide described herein, a vector described herein, a recombinant viral particle described herein, or a recombinant AAV particle described herein.

In one aspect, provided herein is a population of host cells stably transduced by a recombinant viral particle described herein or a recombinant AAV particle described herein.

In one aspect, provided herein is a pharmaceutical composition comprising a recombinant viral particle described herein or a recombinant AAV particle described herein, and a pharmaceutically acceptable carrier.

In one aspect, provided herein is a pharmaceutical composition comprising a population of host cells described herein and a pharmaceutically acceptable carrier.

In one aspect, provided herein is a method of producing a recombinant viral particle or a recombinant AAV particle, comprising culturing a host cell described herein.

In one aspect, provided herein is a method of treating a disease or disorder in a subject in need thereof, comprising administering to the subject a recombinant viral particle described herein, a recombinant AAV particle described herein, or a pharmaceutical composition described herein. In certain embodiments, the disease or disorder is a lysosomal storage disorder. In a specific embodiment, the disease or disorder is Fabry disease. In specific embodiments, the subject is a human.

### Illustrative Embodiments

The present disclosure includes the following non-limiting illustrative embodiments:
1. A polynucleotide comprising a nucleotide sequence encoding human α-galactosidase A, wherein the nucleotide sequence encoding human α-galactosidase A comprises or is set forth in SEQ ID NO: 1.
2. The polynucleotide of embodiment 1, wherein the nucleotide sequence encoding human α-galactosidase A comprises SEQ ID NO:1.
3. The polynucleotide of embodiment 1, wherein the nucleotide sequence encoding human α-galactosidase A is set forth in SEQ ID NO:1.
4. A polynucleotide comprising a nucleotide sequence encoding a promoter, wherein the nucleotide sequence encoding the promoter (i) comprises SEQ ID NO:3, SEQ ID NO:4, or SEQ ID NO:10, or (ii) is set forth in SEQ ID NO:3, SEQ ID NO:4, or SEQ ID NO:10.
5. A polynucleotide comprising a nucleotide sequence encoding human α-galactosidase A and a nucleotide sequence encoding a promoter that is operably linked to the nucleotide sequence encoding human α-galactosidase A, wherein the nucleotide sequence encoding human α-galactosidase A comprises or is set forth in SEQ ID NO:1.
6. The polynucleotide of embodiment 5, wherein the nucleotide sequence encoding human α-galactosidase A comprises SEQ ID NO:1.
7. The polynucleotide of embodiment 5, wherein the nucleotide sequence encoding human α-galactosidase A is set forth in SEQ ID NO:1.
8. The polynucleotide of any one of embodiments 4-7, wherein the promoter is a hepatocyte-specific promoter.
9. The polynucleotide of any one of embodiments 4-8, wherein the promoter is a hepatocyte-myocyte bispecific promoter.
10. The polynucleotide of any one of embodiments 4-9, wherein the nucleotide sequence encoding the promoter comprises SEQ ID NO:3.
11. The polynucleotide of any one of embodiments 4-8, wherein the nucleotide sequence encoding the promoter is set forth in SEQ ID NO:3.
12. The polynucleotide of any one of embodiments 4-9, wherein the nucleotide sequence encoding the promoter comprises SEQ ID NO:4.
13. The polynucleotide of any one of embodiments 4-9, wherein the nucleotide sequence encoding the promoter is set forth in SEQ ID NO:4.
14. The polynucleotide of any one of embodiments 4-9, wherein the nucleotide sequence encoding the promoter comprises SEQ ID NO:10.
15. The polynucleotide of any one of embodiments 4-9, wherein the nucleotide sequence encoding the promoter is set forth in SEQ ID NO:10.
16. A polynucleotide comprising a nucleotide sequence encoding a biologic molecule and a nucleotide sequence encoding a promoter that is operably linked to the nucleotide sequence encoding the biologic molecule, wherein the nucleotide sequence encoding the promoter (i) comprises SEQ ID NO:3, SEQ ID NO:4, or SEQ ID NO:10, or (ii) is set forth in SEQ ID NO:3, SEQ ID NO:4, or SEQ ID NO:10.
17. The polynucleotide of embodiment 16, wherein the promoter is a hepatocyte-specific promoter.
18. The polynucleotide of embodiment 16 or 17, wherein the promoter is a hepatocyte-myocyte bispecific promoter.
19. The polynucleotide of any one of embodiments 16-18, wherein the nucleotide sequence encoding the promoter comprises SEQ ID NO:3.
20. The polynucleotide of embodiment 16 or 17, wherein the nucleotide sequence encoding the promoter is set forth in SEQ ID NO:3.
21. The polynucleotide of any one of embodiments 16-18, wherein the nucleotide sequence encoding the promoter comprises SEQ ID NO:4.
22. The polynucleotide of any one of embodiments 16-18, wherein the nucleotide sequence encoding the promoter is set forth in SEQ ID NO:4.
23. The polynucleotide of any one of embodiments 16-18, wherein the nucleotide sequence encoding the promoter comprises SEQ ID NO:10.
24. The polynucleotide of any one of embodiments 16-18, wherein the nucleotide sequence encoding the promoter is set forth in SEQ ID NO:10.
25. The polynucleotide of any one of embodiments 16-24, wherein the biologic molecule is human α-galactosidase A.
26. The polynucleotide of any one of embodiments 5-25, which further comprises a nucleotide sequence encoding a polyA signal.
27. A polynucleotide comprising a nucleotide sequence encoding an expression cassette, wherein the nucleotide sequence encoding the expression cassette comprises or is set forth in SEQ ID NO:16 or SEQ ID NO:18.
28. The polynucleotide of embodiment 27, wherein the nucleotide sequence encoding the expression cassette comprises SEQ ID NO:16.
29. The polynucleotide of embodiment 27, wherein the nucleotide sequence encoding the expression cassette is set forth in SEQ ID NO:16.
30. The polynucleotide of embodiment 27, wherein the nucleotide sequence encoding the expression cassette comprises SEQ ID NO:18.
31. The polynucleotide of embodiment 27, wherein the nucleotide sequence encoding the expression cassette is set forth in SEQ ID NO:18.
32. A promoter, which is encoded by a nucleotide sequence that (i) comprises SEQ ID NO:3, SEQ ID NO:4, or SEQ ID NO:10, or (ii) is set forth in SEQ ID NO:3, SEQ ID NO:4, or SEQ ID NO:10.
33. The promoter of embodiment 32, which is a hepatocyte-specific promoter.
34. The promoter of embodiment 32 or 33, which is a hepatocyte-myocyte bispecific promoter.
35. The promoter of any one of embodiments 32-34, wherein the nucleotide sequence encoding the promoter comprises SEQ ID NO:3.
36. The promoter of embodiment 32 or 33, wherein the nucleotide sequence encoding the promoter is set forth in SEQ ID NO:3.
37. The promoter of any one of embodiments 32-34, wherein the nucleotide sequence encoding the promoter comprises SEQ ID NO:4.
38. The promoter of any one of embodiments 32-34, wherein the nucleotide sequence encoding the promoter is set forth in SEQ ID NO:4.
39. The promoter of any one of embodiments 32-34, wherein the nucleotide sequence encoding the promoter comprises SEQ ID NO:10.
40. The promoter of any one of embodiments 32-34, wherein the nucleotide sequence encoding the promoter is set forth in SEQ ID NO:10.
41. An expression cassette comprising a nucleotide sequence encoding human α-galactosidase A and a nucleotide sequence encoding a promoter that is operably linked to the nucleotide sequence encoding human α-galactosidase A, wherein the nucleotide sequence encoding human α-galactosidase A comprises or is set forth in SEQ ID NO:1.
42. The expression cassette of embodiment 41, wherein the nucleotide sequence encoding human α-galactosidase A comprises SEQ ID NO:1.
43. The expression cassette of embodiment 41, wherein the nucleotide sequence encoding human α-galactosidase A is set forth in SEQ ID NO:1.
44. The expression cassette of any one of embodiments 41-43, wherein the promoter is a hepatocyte-specific promoter.
45. The expression cassette of any one of embodiments 41-44, wherein the promoter is a hepatocyte-myocyte bispecific promoter.
46. The expression cassette of any one of embodiments 41-45, wherein the nucleotide sequence encoding the promoter comprises SEQ ID NO:3.
47. The expression cassette of any one of embodiments 41-44, wherein the nucleotide sequence encoding the promoter is set forth in SEQ ID NO:3.
48. The expression cassette of any one of embodiments 41-45, wherein the nucleotide sequence encoding the promoter comprises SEQ ID NO:4.
49. The expression cassette of any one of embodiments 41-45, wherein the nucleotide sequence encoding the promoter is set forth in SEQ ID NO:4.
50. The expression cassette of any one of embodiments 41-45, wherein the nucleotide sequence encoding the promoter comprises SEQ ID NO:10.
51. The expression cassette of any one of embodiments 41-45, wherein the nucleotide sequence encoding the promoter is set forth in SEQ ID NO:10.
52. An expression cassette comprising a nucleotide sequence encoding a biologic molecule and a nucleotide sequence encoding a promoter that is operably linked to the nucleotide sequence encoding the biologic molecule, wherein the nucleotide sequence encoding the promoter (i) comprises SEQ ID NO:3, SEQ ID NO:4, or SEQ ID NO:10, or (ii) is set forth in SEQ ID NO:3, SEQ ID NO:4, or SEQ ID NO:10.
53. The expression cassette of embodiment 52, wherein the promoter is a hepatocyte-specific promoter.
54. The expression cassette of embodiment 52 or 53, wherein the promoter is a hepatocyte-myocyte bispecific promoter.
55. The expression cassette of any one of embodiments 52-54, wherein the nucleotide sequence encoding the promoter comprises SEQ ID NO:3.
56. The expression cassette of embodiment 52 or 53, wherein the nucleotide sequence encoding the promoter is set forth in SEQ ID NO:3.
57. The expression cassette of any one of embodiments 52-54, wherein the nucleotide sequence encoding the promoter comprises SEQ ID NO:4.
58. The expression cassette of any one of embodiments 52-54, wherein the nucleotide sequence encoding the promoter is set forth in SEQ ID NO:4.
59. The expression cassette of any one of embodiments 52-54, wherein the nucleotide sequence encoding the promoter comprises SEQ ID NO:10.
60. The expression cassette of any one of embodiments 52-54, wherein the nucleotide sequence encoding the promoter is set forth in SEQ ID NO:10.
61. The expression cassette of any one of embodiments 52-60, wherein the biologic molecule is human α-galactosidase A.
62. The expression cassette of any one of embodiments 41-61, which further comprises a nucleotide sequence encoding a polyA signal.
63. An expression cassette, which is encoded by a nucleotide sequence that (i) comprises SEQ ID NO:16 or SEQ ID NO:18, or (ii) is set forth in SEQ ID NO:16 or SEQ ID NO:18.
64. The expression cassette of embodiment 63, wherein the nucleotide sequence encoding the expression cassette comprises SEQ ID NO. 16.
65. The expression cassette of embodiment 63, wherein the nucleotide sequence encoding the expression cassette is set forth in SEQ ID NO. 16.
66. The expression cassette of embodiment 63, wherein the nucleotide sequence encoding the expression cassette comprises SEQ ID NO. 18.
67. The expression cassette of embodiment 63, wherein the nucleotide sequence encoding the expression cassette is set forth in SEQ ID NO. 18.
68. A polynucleotide comprising a nucleotide sequence encoding the expression cassette of any one of embodiments 41-67.
69. A vector comprising the polynucleotide of any one of embodiments 1-31 and 68, the promoter of any one of embodiments 32-40, or the expression cassette of any one of embodiments 41-67.
70. A recombinant viral particle comprising a recombinant viral genome comprising the expression cassette of any one of embodiments 41-67.
71. A recombinant adeno-associated virus (AAV) particle comprising: (a) an AAV capsid; and (b) a recombinant AAV genome comprising the expression cassette of any one of embodiments 41-67 flanked by AAV-inverted terminal repeats (ITRs).
72. The recombinant AAV particle of embodiment 71, which is a recombinant AAV serotype 9 (rAAV9) particle.
73. The recombinant AAV particle of embodiment 71 or 72, wherein the AAV capsid comprises a variant AAV9 capsid protein.
74. The recombinant AAV particle of embodiment 73, wherein the variant AAV9 capsid protein comprises the amino acid sequence of SEQ ID NO:31, 38, 49, 51, 53, 55, 57, 59 or 61.
75. The recombinant AAV particle of embodiment 74, wherein the variant AAV9 capsid protein comprises the amino acid sequence of SEQ ID NO:30, 37, 48, 50, 52, 54, 56, 58 or 60.
76. A host cell comprising the polynucleotide of any one of embodiments 1-31 and 68, the vector of embodiment 69, the recombinant viral particle of embodiment 70, or the recombinant AAV particle of any one of embodiments 71-75.
77. A population of host cells stably transduced by the recombinant viral particle of embodiment 70 or the recombinant AAV particle of any one of embodiments 71-75.
78. A pharmaceutical composition comprising the recombinant viral particle of embodiment 70 or the recombinant AAV particle of any one of embodiments 71-75, and a pharmaceutically acceptable carrier.
79. A pharmaceutical composition comprising the population of host cells of embodiment 77 and a pharmaceutically acceptable carrier.
80. A method of producing a recombinant viral particle or a recombinant AAV particle, comprising culturing the host cell of embodiment 76.
81. A method of treating a disease or disorder in a subject in need thereof, comprising administering to the subject the recombinant viral particle of embodiment 70, the recombinant AAV particle of any one of embodiments 71-75, or the pharmaceutical composition of embodiment 78 or 79.
82. The method of embodiment 81, wherein the disease or disorder is a lysosomal storage disorder.
83. The method of embodiment 81, wherein the disease or disorder is Fabry disease.
84. The method of any one of embodiments 81-83, wherein the subject is a human.

### BRIEF DESCRIPTION OF THE FIGURES

FIG. 1. Human α-galactosidase A (GLA) codon optimization. FIG. 1A: ELISA assay result. FIG. 1B: α-galactosidase A activity result. FIG. 1C: Western blot analysis of cell culture supernatant from HepG2 transfected with hGLA-encoding plasmids. WT: wild type; hGLA: human alpha-galactosidase A.

FIG. 2. Hepatocyte-cardiomyocyte (HC) bi-specific promoters in vitro testing. The RLuc/FLuc ratio was used for measuring the promoter strength of five promoters (HC1-HC5). RLuc: Renilla luciferase; FLuc:Firely luciferase.

FIG. 3. Truncated mouse muscle creatine kinase (MCK) promoter in vitro testing. FIG. 3A: The RLuc/FLuc ratio was used for measuring the promoter strength of three versions of MCK promoter (MCK-D1~MCK-D3). FIGS. 3B and 3C: The activity of promoter HC7 was evaluated by in vivo imaging system (IVIS). The strong luminescence signals in the liver and skeletal muscles were observed one week (FIG. 3B) and three weeks (FIG. 3C) after injection.

FIG. 4. Human GLA expression cassettes mediated α-galactosidase A expression (FIG. 4A) and activity (FIG. 4B) in HepG2. FIG. 4A: α-Gal A concentrations for negative control, Cassette 1, Cassette 2, Cassette 3, and Cassette 4 are: 2 ng/mL, 2993 ng/mL, 2225 ng/mL, 1919 ng/mL, and 1684 ng/mL, respectively. FIG. 4B: α-Gal A activities for negative control, Cassette 1, Cassette 2, Cassette 3, and Cassette 4 are: 1.66×10⁶ nmol/mg/h, 1.87×10⁶ nmol/mg/h, 2.00×10⁶ nmol/mg/h, and 2.73×10⁶ nmol/mg/h, respectively.

FIG. 5. Human GLA expression cassettes mediated α-galactosidase A expression (FIG. 5A) and activity (FIG. 5B) in H9c2 cells. FIG. 5A: α-Gal A concentrations for negative control, Cassette 1, Cassette 2, Cassette 3, and Cassette 4 are: 2×10⁻¹ ng/mL, 306 ng/mL, 249 ng/mL, 954 ng/mL, and 1350 ng/mL, respectively. FIG. 5B: α-Gal A activities for negative control, Cassette 1, Cassette 2, Cassette 3, and Cassette 4 are: 1.14×10⁴ nmol/mg/h, 1.45×10⁴ nmol/mg/h, 1.80×10⁴ nmol/mg/h, and 2.57×10⁴ nmol/mg/h, respectively.

FIGS. 6A-6J. Characterization of Cassette 1-mediated activity and efficacy in Fabry mouse model. FIG. 6A: Serum α-Gal A activity over time. FIG. 6B: Serum α-Gal A activity at day 61 (the numbers on top of bars from left to right are: 11, 3, 1959, 25956, 80140, and 203339). FIG. 6C: The hGLA transgene mRNA level in the liver (the numbers on top of bars from left to right are: 0.3200, 2.2752, 3.9636, 9.6976, and 0.0001). FIG. 6D: The hGLA transgene mRNA level in the heart (the numbers on top of bars from left to right are: 3.3037×10-s, 0.0005, 0.0005, and 0.0008). FIG. 6E: The vector genome copies in liver (the numbers on top of bars from left to right are: 0.94, 10.88, 28.28, 79.60, and 0.02). FIG. 6F: The vector genome copies in liver (the numbers on top of bars from left to right are: 0.03, 0.24, 0.87, and 1.64). FIG. 6G: Western-blot results. FIG. 6H: α-Gal A activities in the liver, heart, and kidney. Data for liver shown from the left to right are: 2043 (2E11 vg/kg), 48242 (2E12 vg/kg), 108653 (6E12 vg/kg), 175176 (2E13 vg/kg), 12 (GLA-KO), and 35 (wild-type). Data for heart shown from the left to right are: 31 (2E11 vg/kg), 1186 (2E12 vg/kg), 1533 (6E12 vg/kg), 12153 (2e13 vg/kg), 26 (GLA-KO), and 10 (wild-type). Data for kidney shown from the left to right are: 18 (2E11 vg/kg), 217 (2E12 vg/kg), 501 (6E12 vg/kg), 1821 (2E13 vg/kg), 6 (GLA-KO), and 21 (wild-type). FIG. 6I: Serum lyso-Gb3 level (the numbers on top of bars from left to right are: 0.57, 147.22, 3.67, 0.76, 0.59, and 0.55). FIG. 6J: The lyso-Gb3 levels in the liver, heart and kidney.

FIGS. 7A-7E. Characterization of AAV-hGLA mediated activity and efficacy in Fabry mouse model. FIG. 7A: Serum α-GAL A activity over time. FIG. 7B: The vector genome copies in the liver, heart, and quadriceps. FIG. 7C: The hGLA transgene mRNA levels in the liver, heart and quadriceps. FIG. 7D: Western-blot results. FIG. 7E: Cassette 3-mediated α-Gal A activities in the liver, heart and kidney. FIG. 7F: Male 8-week-old G1α-ko mice were intravenously dosed with HC7-driven hGLAcoI (Cassette 3) packaged with capsid AVT908. The dose was 2e12 vg/kg. Eight weeks after dosing, the liver, heart and kidney were harvested and processed for immunohistochemistry (IHC) staining. The rabbit anti-human GLA antibody (Sigma-Aldrich, Cat.Nol. HPA000237) was diluted at 1:1000 and used as primary antibody. The Peroxidase AffmiPure Goat Anti-Rabbit IgG (H+L) (jacksonimmuno, 111-035-003) was diluted by 1000-fold and used as detection antibody. The DAB was used as substrate. The brown color indicates human Gal A protein positive staining.

FIGS. 8A-8B. Adult male Cynomolgus monkeys were intravenously administrated with hGLA expression cassette 3 packaged in capsid AVT917, AVT918 or AAV9 at the dose of 2E13 vg/kg (N=1/capsid). The plasma was collected before and after the administration. FIG. 8A: The Gal A activities in the plasma were measured by using fluorescence-based 4-MU method. FIG. 8A: The expressed human Gal A protein in the plasma were measured by using ELISA.

FIG. 9A. AAV vector genome biodistribution in NHP. An adult male Cynomolgus monkeys was intravenously administrated with AVT917 containing hGLA expression cassette 3 at the dose of 2E13 vg/kg. Thirteen weeks after the dosing, the tissues were collected and processed for vector genome copy analysis by using ddPCR. The RNase P gene was used as a reference.

FIG. 9B. AVT917 and HC7 promoter mediated hGLA transgene expression in NHP. An adult male Cynomolgus monkeys was intravenously administrated with AVT917 containing hGLA expression cassette 3 at the dose of 2E13 vg/kg. Thirteen weeks after the dosing, the tissues were collected and processed for transgene mRNA level analysis by using RT-qPCR. The GADPH gene was used as a reference. The data was expressed as 2^{-ΔCt}.

FIG. 9C. The −Gal A activities in the tissue of NHP treated with AVT917-hGLA. An adult male Cynomolgus monkeys was intravenously administrated with AVT917 containing hGLA expression cassette 3 at the dose of 2E13 vg/kg. Thirteen weeks after the dosing, the tissues were collected and processed for −Gal A activities analysis by using fluorescence-based 4-MU method.

FIG. 10. AVT917 mediated high hGLA expression in the Fabry mice. Adult (16-week-old) male Fabry mice (Gla-KO) were intravenously injected with vehicle (formulation buffer) or AVT919-HC7-hGLA (cassette 3) at 3 doses (n=10/group). The plasma was collected bi-weekly for α-Gal A activity measurements. Data were show as Mean with SD. **p<0.01, *** p<0.001, ****p<0.0001, two-way repeated ANOVA followed by Tukey's multiple comparisons test.

FIG. 11. AVT917 mediated hGLA mRNA expression in the tissues of Fabry mice. Adult (16-week-old) male Fabry mice (Gla-KO) were intravenously injected with vehicle (formulation buffer) or AVT919-HC7-hGLA (cassette 3) at 3 doses (n=10/group). Eight weeks after dosing, the major tissues were collected and processed for total RNA isolation. The hGLA specific mRNA transcripts were quantified by RT-qPCR1. The mouse GAPDH (MsGAPDH) was used as housekeeping gene. Data were show as Mean with SD. *p<0.05, ****p<0.0001, one-way ANOVA followed by Tukey's multiple comparisons test.

FIG. 12. Significant substrate reduction in Fabry mice treated with AVT917-HC7-hGLA. Adult (16-week-old) male Fabry mice (Gla-KO) were intravenously injected with vehicle (formulation buffer) or AVT919-HC7-hGLA (cassette 3) at 3 doses (n=10/group). Eight weeks after dosing, the major tissues were collected and processed for Lyso-Gb3 quantification by using LC-MS/MS. Data were expressed as Mean with SD. The percentage above the bar represents the relative Lyso-Gb3 level and the level in the vehicle group was set as 100%. *p<0.05, **p<0.01, *** p<0.001, **** p<0.0001, one-way ANOVA followed by Tukey's multiple comparisons test; #p<0.0001, unpaired t-test.

FIG. 13A. AVT917 mediated hGLA expression in the NHPs. Adult male Cynomolgus monkeys were intravenously administrated with AVT917-HC7-hGLA at the dose of 2E12 vg/kg (N=3) and 6E12 vg/kg (N=2). The plasma was collected before and after the administration. The α-Gal A activities in the plasma were measured by using fluorescence-based 4-MU method.

FIG. 13B. Adult male Cynomolgus monkeys were intravenously administrated with AVT917-HC7-hGLA at the dose of 2E12 vg/kg (N=3) and 6E12 vg/kg (N=2). The plasma was collected before and after the administration. The expressed human α-Gal A protein in the plasma was measured by using ELISA.

FIG. 14. Adult male Cynomolgus monkeys were intravenously administrated with AVT917 or AVT919 containing cassette 3 (SEQ ID:18) at the dose of 2E12 vg/kg (N=1/capsid). The plasma was collected for human α-Gal A protein concentration measurements by ELISA.

### DETAILED DESCRIPTION

The present disclosure describes codon-optimized nucleotide sequences encoding human α-galactosidase A, and describes data suggesting that these nucleotide sequences are particularly suitable for gene therapies for Fabry disease. The present disclosure also describes nucleotide sequences encoding a hepatocyte-specific or hepatocyte-myocyte bispecific promoter, and describes data suggesting that these nucleotide sequences are particular suitable for gene therapies, especially for lysosomal storage disorders including Fabry disease. The present disclosure further describes nucleotide sequences encoding a human α-galactosidase A expression cassette, and describes data suggesting that these nucleotide sequences are particularly suitable for gene therapies for Fabry disease. The present disclosure provides polynucleotides, promoters, expression cassettes, vectors, host cells, gene delivery systems (e.g., recombinant viral particles such as recombinant adeno-associated virus (AAV) particles and non-viral gene delivery systems) and pharmaceutical compositions related to these nucleotide sequences, as well as methods using the same.

### 5.1 Definitions

Techniques and procedures described or referenced herein include those that are generally well understood and/or commonly employed using conventional methodology by those skilled in the art, such as, for example, the widely utilized methodologies described in Green and Sambrook, Molecular Cloning: A Laboratory Manual, 4^{th} Ed., Cold Spring Harbor Laboratory (Cold Spring Harbor, N.Y. 2012); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dibel eds., 2d ed. 2010).

Unless otherwise defined herein, technical and scientific terms used in the present description have the meanings that are commonly understood by those of ordinary skill in the art. For purposes of interpreting this specification, the following description of terms will apply.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art.

“Polynucleotide” or “nucleic acid,” as used interchangeably herein, refers to polymers of nucleotides of any length and includes DNA (including cDNA) and RNA (including mRNA). The nucleotides can be deoxyribonucleotides, ribonucleotides, modified nucleotides or bases, and/or their analogs, or any substrate that can be incorporated into a polymer by DNA or RNA polymerase or by a synthetic reaction. The polynucleotide can be single-stranded or double-stranded. The polynucleotide can be linear or circular. A polynucleotide may comprise modified nucleotides, such as methylated nucleotides and their analogs. “Oligonucleotide,” as used herein, refers to short synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.” In certain embodiments, a polynucleotide described herein can be optimized by alternative or preferred codon usage for a particular host cell or delivery target cell type. Codon optimization can be performed using any suitable method known in the art (e.g., using a suitable software known in the art).

An “isolated polynucleotide” or “isolated nucleic acid” as used herein is a nucleic acid, for example, an RNA, DNA, or a mixed nucleic acids, which is usually substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a polypeptide described herein is a nucleic acid molecule that is identified and separated from at least one contaminant nucleic acid molecule with which it is ordinarily associated in the environment in which it was produced.

The term “homology” as used herein refers to the percent identity between two polynucleotide or two polypeptide moieties. Two DNA, or two polypeptide sequences are “substantially homologous” to each other when the sequences exhibit at least about 50%, at least about 75%, at least about 80%-85%, at least about 90%, at least about 95%-98% sequence identity, at least about 99%, or any percent therebetween over a defined length of the molecules. As used herein, substantially homologous also refers to sequences showing complete identity to the specified DNA or polypeptide sequence.

The term “identity” or “identical” as used herein refers to an exact nucleotide-to-nucleotide or amino acid-to-amino acid correspondence of two polynucleotides or polypeptide sequences, respectively. Methods for determining percent identity are well known in the art. For example, percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions. Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters:genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are well known in the art. Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The term “vector” as used herein refers to a substance that is used to carry or include a nucleic acid sequence, for example, in order to introduce a nucleic acid sequence into a host cell. Vectors applicable for use include, for example, expression vectors, plasmids, bacmids, cosmids, constructs, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed both nucleic acid molecules can be inserted, for example, into a single expression vector or in separate expression vectors. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. The term “vector” includes cloning and expression vehicles, as well as viral vectors. In certain embodiments, the vector provided herein is a recombinant viral vector. In specific embodiments, the vector provided herein is a recombinant AAV vector.

The term “recombinant AAV vector (rAAV vector)” as used herein refers to a polynucleotide vector comprising a nucleic acid sequence from an AAV and one or more heterologous sequences (i.e., nucleic acid sequence not of AAV origin). In some embodiments, the one or more heterologous sequences are flanked by at least one, in certain embodiments two, AAV inverted terminal repeat sequences (ITRs). In some embodiments, such rAAV vectors can be replicated and packaged into infectious viral capsid particles, e.g., when present in a host cell that has been infected with a suitable helper virus (or that is expressing suitable helper functions) and that is expressing AAV rep and cap gene products (i.e., AAV Rep and Cap proteins). An rAAV vector may be incorporated into a larger polynucleotide (e.g., in a chromosome or in another vector such as a plasmid used for cloning or transfection), and can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral capsid particle, particularly an AAV particle. An rAAV vector can be packaged into an AAV capsid to generate a “recombinant adeno-associated viral capsid particle (rAAV particle).”

The term “heterologous” as used herein in connection with nucleic acid sequences such as coding sequences and control sequences, refers to sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene).

The term “flanked” as used herein with respect to a sequence that is flanked by other elements, indicates the presence of one or more the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence (e.g., a transgene) that is “flanked” by two other elements (e.g., ITRs) indicates that one element is located 5′ to the sequence and the other is located 3′ to the sequence; however, there may be intervening sequences therebetween.

The term “inverted terminal repeat” or “ITR” sequence as used herein refers to relatively short sequences found at the termini of viral genomes which are in opposite orientation. An “AAV inverted terminal repeat (ITR)” sequence is well known in the art, and is usually an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contain several shorter regions of self-complementarity (designated A, A′, B, B′, C, C′ and D regions), allowing intrastrand base-pairing to occur within this portion of the ITR.

A “coding sequence” or a sequence which “encodes” a selected polypeptide, is a nucleic acid molecule which is transcribed (in the case of DNA) and translated (in the case of mRNA) into a polypeptide when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the terms “operably linked,” “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

As used herein the term “expression cassette” or “nucleic acid expression cassette” refers to nucleic acid molecules that include one or more transcriptional control elements (such as, but not limited to promoters, response elements, polyadenylation sequences, and introns) that direct expression of a biologic molecule, e.g., direct (trans)gene expression, in one or more desired cell types, tissues or organs. Typically, they will also contain a transgene, although it is also contemplated that an expression cassette directs expression of an endogenous gene in a cell into which the nucleic acid sequence is inserted.

The term “response element (RE)” as used herein refers to transcriptional control elements, in particular non-coding cis-acting transcriptional control elements, capable of regulating and/or controlling transcription of a gene. Response elements comprise at least one transcription factor binding site (TFBS). Response elements may be situated either upstream (e.g. in the promoter region) or downstream (e.g. in the 3′ UTR) of the gene they regulate in vivo, and may be located in the immediate vicinity of the gene or further away. Of note, response elements can be naturally occurring sequences or non-naturally occurring sequences.

The term “enhancer” as used herein refers to a nucleic acid sequence that increases the rate of transcription by increasing the activity of a promoter.

The term “promoter” as used herein in its ordinary sense refers to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters.”

The term “TATA-box” as used herein refers to a DNA sequence found in the promoter region of a gene that can be bound by TATA binding protein and transcription factor II D during DNA unwinding and binding by RNA polymerase II. A TATA box sequence typically includes a TATAAA sequence and often includes additional 3′ adenine nucleotides.

The term “transcription start site” as used herein refers to the location where transcription starts at the 5′ end of a gene sequence.

The term “ORF (open reading frame)” as used herein refers to a series ofnucleotide triplets (codons) coding for amino acids. These sequences are usually translatable into a peptide.

The term “transcription factor (TF)” as used herein refers to a protein that binds to specific DNA sequences and thereby controls the transfer (or transcription) of genetic information from DNA to RNA. TFs perform this function alone or with other proteins in a complex, by promoting (as an activator), or blocking (as a repressor) the recruitment of RNA polymerase (the enzyme that performs the transcription of genetic information from DNA to RNA) to specific genes. The specific DNA sequences to which a TF binds is known as a response element (RE) or regulatory element. Other names include cis-element and cis-acting transcriptional regulatory element. Transcription factors interact with their binding sites using a combination of electrostatic (of which hydrogen bonds are a special case) and Van der Waals forces. Due to the nature of these chemical interactions, most transcription factors bind DNA in a sequence specific manner. However, not all bases in the transcription factor-binding site may actually interact with the transcription factor. In addition, some of these interactions may be weaker than others. Thus, many transcription factors do not bind just one sequence but are capable of binding a subset of closely related sequences, each with a different strength of interaction. For example, although the consensus binding site for the TATA-binding protein (TBP) is TATAAAA; however, the TBP transcription factor can also bind similar sequences such as TATATAT or TATATAA. Transcription factors (TFs) can be classified based on many aspects. For example, the secondary, tertiary and quaternary structures of the protein structures DNA-binding sequence and properties, the interaction with the double helix of the DNA, and the metal and other binding characteristics. The JASPAR database and TRANSFAC (TRANSFAC. RTM. 7.0 Public 2005) are two web-based transcription factor databases, their experimentally-proven binding sites, and regulated genes.

The term “transgene” as used herein in a broad sense means any heterologous nucleotide sequence incorporated in a vector, such as a viral vector, e.g., for expression in a target cell and it can be associated with expression control sequences, such as promoters. It is appreciated by those of skill in the art that expression control sequences will be selected based on ability to promote expression of the transgene in the target cell. An example of a transgene is a nucleic acid encoding a therapeutic polypeptide or a detectable marker.

The term “AAV capsid” or “AAV capsid protein” or “AAV cap” as used herein refers to a protein encoded by an AAV capsid (cap) gene (e.g., VP1, VP2, and VP3) or a variant thereof. For example, the term includes but is not limited to a capsid protein derived from any AAV serotype such as AAV1, AAV2, AAV2i8, AAV3, AAV3-B, AAV4, AAV5, AAV6, AAV7, AAV8, AAVrh8, AAVrh8R, AAV9, AAV10, AAVrhl0, AAV11, AAV12, AAV13, AAV-DJ, AAV-2/1, AAV 2/6, AAV 2/7, AAV 2/8, AAV 2/9, AAV LK03, AAVrhl0, AAVrh74, AAV44-9, or a variant thereof. The term also includes a capsid protein expressed by or derived from a recombinant AAV such as a chimeric AAV.

The term “AAV capsid particle” or “AAV particle” as used herein includes at least one AAV capsid protein (e.g., a VP1 protein, a VP2 protein, a VP3 protein, or variant thereof) and optionally encapsulates a nucleic acid from an AAV genome (including an rAAV genome) or a nucleic acid derived from an AAV genome (including an rAAV genome).

The term “serotype” used with respect to vector or virus capsid is defined by a distinct immunological profile based on the capsid protein sequences and capsid structure.

The term “chimeric” as used herein means, with respect to a viral capsid or particle, that the capsid or particle includes sequences from different parvoviruses, preferably different AAV serotypes, as described in Rabinowitz et al., U.S. Pat. No. 6,491,907, the disclosure of which is incorporated in its entirety herein by reference.

The term “recombinant” means a genetic entity distinct from that generally found in nature. As applied to a polynucleotide, gene, promoter, expression cassette, or the like, this means that it is the product of various combinations of cloning, restriction, ligation and/or synthesis steps, and other procedures that result in the production of a construct that is distinct from a polynucleotide, gene, promoter, expression cassette, or the like found in nature. The term “recombinant” when used herein to describe a virus refers to a virus that has been genetically altered, e.g., by the addition or insertion of a heterologous nucleic acid construct into the particle. For example, the term “recombinant AAV particle” or “rAAV” as used herein refers to an AAV that has been genetically altered, e.g., by the deletion or other mutation of an endogenous AAV gene and/or the addition or insertion of a heterologous nucleic acid construct into the polynucleotide of the AAV particle.

As used herein, “specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

As used herein, “stringent hybridization conditions” or “stringent conditions” refers to conditions under which an oligomeric compound will hybridize to its target sequence, but to a minimal number of other sequences.

The term “transfected” or “transformed” or “transduced” as used herein refers to a process by which exogenous nucleic acid is transferred or introduced into a host cell. A “transfected” or “transformed” or “transduced” cell is one which has been transfected, transformed or transduced with exogenous nucleic acid. For example, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Green and Sambrook, Molecular Cloning: A Laboratory Manual, 4^{th} Ed., Cold Spring Harbor Laboratory (Cold Spring Harbor, N.Y. 2012), Davis et al. (1986) Basic Methods in Molecular Biology, Elsevier, and Chu et al. (1981) Gene 13:197. Such techniques can be used to introduce one or more exogenous molecules into suitable host cells. “Transduction” of a cell by a virus means that there is transfer of a nucleic acid such as DNA or RNA from the virus particle to the cell.

The term “host cell” as used herein refers to a particular cell that may be transfected with a nucleic acid molecule, vector, viral particle, or non-viral gene delivery system, and the progeny or potential progeny of such a cell. Host cells may be bacterial cells, yeast cells, insect cells or mammalian cell.

The term “purified” refers to isolation of a substance (compound, polynucleotide, protein, polypeptide, polypeptide composition) such that the substance of interest comprises the majority percent of the sample in which it resides. Typically, in a sample a substantially purified component comprises 50%, 80%-85%, 90-99%, such as at least 90%, 91%, 92%, 93%, 94%, 95%, 96%, 97%, 98%, 99% of the sample. Techniques for purifying polynucleotides and polypeptides of interest are well-known in the art and include, for example, ion-exchange chromatography, affinity chromatography and sedimentation according to density.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease. “Treatment” or “treating” includes: (1) preventing the disease, i.e., preventing the development of the disease or causing the disease to occur with less intensity in a subject that may be exposed to or predisposed to the disease but does not yet experience or display symptoms of the disease, (2) inhibiting the disease, i.e., arresting the development, preventing or retarding progression, or reversing the disease state, (3) relieving symptoms of the disease i.e., decreasing the number of symptoms experienced by the subject, and (4) reducing, preventing or retarding progression of the disease or a symptom thereof. The terms “prevent,” “preventing,” and “prevention” refer to reducing the likelihood of the onset (or recurrence) of a disease, disorder, condition, or associated symptom(s).

As used herein, “administer”, “administration”, or “administering” refers to the act of injecting or otherwise physically delivering a substance (e.g., a pharmaceutical composition provided herein) to a subject or a patient (e.g., human), such as by oral, mucosal, topical, intradermal, parenteral, intravenous, intravitreal, intraarticular, subretinal, intramuscular, intrathecal delivery and/or any other method of physical delivery described herein or known in the art. In a particular embodiment, administration is by intravenous infusion. A therapeutic (e.g., a pharmaceutical composition) provided herein may be delivered systemically or to a specific tissue.

As used herein, the terms “effective amount” or “therapeutically effective amount” refer to an amount of a therapeutic (e.g., a pharmaceutical composition provided herein) which is sufficient to treat, diagnose, prevent, delay the onset of, reduce and/or ameliorate the severity and/or duration of a given condition, disorder or disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy. In some embodiments, “effective amount” as used herein also refers to the amount of a therapeutic (e.g., a pharmaceutical composition provided herein) described herein to achieve a specified result.

As used herein, the terms “subject” and “patient” are used interchangeably and refer to a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder described herein. In another embodiment, the subject is a mammal, e.g., a human, at risk of developing a disease or disorder described herein. In a specific embodiment, the subject is human. In a specific embodiment, the subject is a human adult. In a specific embodiment, the subject is a human adolescent. In a specific embodiment, the subject is a human child.

As used herein, the terms “therapies” and “therapy” can refer to any protocol(s), method(s), compositions, formulations, and/or agent(s) that can be used in the prevention, treatment, management, or amelioration of a disease or disorder or symptom thereof (e.g., a disease or disorder described herein or one or more symptoms or condition associated therewith). In certain embodiments, the terms “therapies” and “therapy” refer to gene therapies useful in treatment, management, prevention, or amelioration of a disease or disorder or one or more symptoms thereof.

As used herein, the terms “gene therapies” and “gene therapy” refer to the introduction of a heterologous nucleic acid molecule into one or more recipient cells, wherein the heterologous nucleic acid itself or the expression of the heterologous nucleic acid in the recipient cell affects the cell's function and results in a therapeutic effect in a subject. For example, the heterologous nucleic acid molecule may encode a protein, which affects a function of the recipient cell.

As used in this disclosure and unless otherwise specified, the terms “about” and “approximately” shall be construed so as to allow normal variation as judged by a person of skill in the art, or to mean within an acceptable error range for the particular value as determined by one of ordinary skill in the art, which will depend in part on how the value is measured or determined (i.e., the limitations of the measurement system), such as, for example, a variation within 20% or 10% or 5% of the stated value, or within 1 or more than 1 standard deviations per practice in the art. In specific embodiments, the terms “about” and “approximately” encompass the exact value recited. Unless the context clearly dictates otherwise, all numerical values provided herein are modified by the term about.

As used in this disclosure, the singular forms “a”, “an” and “the” include plural referents unless the context clearly dictates otherwise. The terms “a” (or “an”), as well as the terms “one or more,” and “at least one” can be used interchangeably herein unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “or” and “and” can be used interchangeably and can be understood to mean “and/or” unless the context clearly dictates otherwise.

### 5.2 Polynucleotides, Promoters, and Expression Cassettes

The present disclosure describes codon-optimized nucleotide sequences encoding human α-galactosidase A. The human ca-galactosidase A encoded by a nucleotide sequence described in this disclosure can be a wild-type human α-galactosidase A or a mutated (e.g., truncated) human α-galactosidase A, but is preferably a functional human α-galactosidase A. In specific embodiments, the codon-optimized nucleotide sequence encoding human α-galactosidase A comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:1. In one embodiment, the codon-optimized nucleotide sequence encoding human α-galactosidase A comprises SEQ ID NO:1. In one embodiment, the codon-optimized nucleotide sequence encoding human α-galactosidase A is set forth in SEQ ID NO:1. In specific embodiments, the codon-optimized nucleotide sequence encoding human α-galactosidase A comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:2. In one embodiment, the codon-optimized nucleotide sequence encoding human α-galactosidase A comprises SEQ ID NO:2. In specific embodiments, the codon-optimized nucleotide sequence encoding human α-galactosidase A comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:20. In one embodiment, the codon-optimized nucleotide sequence encoding human α-galactosidase A comprises SEQ ID NO:20. In one embodiment, the codon-optimized nucleotide sequence encoding human α-galactosidase A is set forth in SEQ ID NO:20. Without wishing to be bound by any one theory, these nucleotide sequences are particularly suitable for gene therapies for Fabry disease.

The present disclosure also describes nucleotide sequences encoding a hepatocyte-specific or hepatocyte-myocyte bispecific promoter. In certain embodiments, the nucleotide sequence encodes a hepatocyte-specific promoter. In certain embodiments, the nucleotide sequence encodes a hepatocyte-myocyte bispecific promoter.

In specific embodiments, the nucleotide sequence encoding the hepatocyte-specific or hepatocyte-myocyte bispecific promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:3. In one embodiment, the nucleotide sequence encoding the hepatocyte-specific or hepatocyte-myocyte bispecific promoter comprises SEQ ID NO:3. In one embodiment, the nucleotide sequence encoding the hepatocyte-specific or hepatocyte-myocyte bispecific promoter is set forth in SEQ ID NO:3. Without wishing to be bound by any one theory, these nucleotide sequences are particularly suitable for directing expression of a biologic molecule, e.g., directing (trans)gene expression, in hepatocytes (i.e., in the liver) and optionally in other cell types (e.g., myocytes and/or kidney cells), and thus are particularly suitable for gene therapies, especially for lysosomal storage disorders including Fabry disease.

In specific embodiments, the nucleotide sequence encoding the hepatocyte-specific or hepatocyte-myocyte bispecific promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:4. In one embodiment, the nucleotide sequence encoding the hepatocyte-specific or hepatocyte-myocyte bispecific promoter comprises SEQ ID NO:4. In one embodiment, the nucleotide sequence encoding the hepatocyte-specific or hepatocyte-myocyte bispecific promoter is set forth in SEQ ID NO:4. In specific embodiments, the nucleotide sequence encoding the hepatocyte-specific or hepatocyte-myocyte bispecific promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:5. In one embodiment, the nucleotide sequence encoding the hepatocyte-specific or hepatocyte-myocyte bispecific promoter comprises SEQ ID NO:5. In one embodiment, the nucleotide sequence encoding the hepatocyte-specific or hepatocyte-myocyte bispecific promoter is set forth in SEQ ID NO:5. In specific embodiments, the nucleotide sequence encoding the hepatocyte-specific or hepatocyte-myocyte bispecific promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:6. In one embodiment, the nucleotide sequence encoding the hepatocyte-specific or hepatocyte-myocyte bispecific promoter comprises SEQ ID NO:6. In one embodiment, the nucleotide sequence encoding the hepatocyte-specific or hepatocyte-myocyte bispecific promoter is set forth in SEQ ID NO:6. In specific embodiments, the nucleotide sequence encoding the hepatocyte-specific or hepatocyte-myocyte bispecific promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:7. In one embodiment, the nucleotide sequence encoding the hepatocyte-specific or hepatocyte-myocyte bispecific promoter comprises SEQ ID NO:7. In one embodiment, the nucleotide sequence encoding the hepatocyte-specific or hepatocyte-myocyte bispecific promoter is set forth in SEQ ID NO:7. In specific embodiments, the nucleotide sequence encoding the hepatocyte-specific or hepatocyte-myocyte bispecific promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:8. In one embodiment, the nucleotide sequence encoding the hepatocyte-specific or hepatocyte-myocyte bispecific promoter comprises SEQ ID NO:8. In one embodiment, the nucleotide sequence encoding the hepatocyte-specific or hepatocyte-myocyte bispecific promoter is set forth in SEQ ID NO:8. In specific embodiments, the nucleotide sequence encoding the hepatocyte-specific or hepatocyte-myocyte bispecific promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:9. In one embodiment, the nucleotide sequence encoding the hepatocyte-specific or hepatocyte-myocyte bispecific promoter comprises SEQ ID NO:9. In one embodiment, the nucleotide sequence encoding the hepatocyte-specific or hepatocyte-myocyte bispecific promoter is set forth in SEQ ID NO:9. In specific embodiments, the nucleotide sequence encoding the hepatocyte-specific or hepatocyte-myocyte bispecific promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:10. In one embodiment, the nucleotide sequence encoding the hepatocyte-specific or hepatocyte-myocyte bispecific promoter comprises SEQ ID NO:10. In one embodiment, the nucleotide sequence encoding the hepatocyte-specific or hepatocyte-myocyte bispecific promoter is set forth in SEQ ID NO:10. Without wishing to be bound by any one theory, these nucleotide sequences are particularly suitable for directing expression of a biologic molecule, e.g., directing (trans)gene expression, in hepatocytes (i.e., in the liver) and in myocytes (i.e., in the muscle) (for example, in the heart muscle (e.g., heart atrium muscle, and/or heart ventricle muscle) and/or the skeletal muscle (e.g., biceps, triceps, quadriceps, tibialis anterior, gastrocnemius, rectus abdominis, diaphragm, pectoralis major)) and optionally in other cell types (e.g., kidney cells), and thus are particularly suitable for gene therapies, especially for lysosomal storage disorders including Fabry disease. In particular embodiments, muscle can function as a production factory for a biologic molecule, which is secreted from muscle cells and eventually taken up by the cells of another tissue or organ to exert its therapeutic effect.

The present disclosure further describes nucleotide sequences encoding a human α-galactosidase A expression cassette. In specific embodiments, the nucleotide sequence encoding the human α-galactosidase A expression cassette comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:16. In one embodiment, the nucleotide sequence encoding the human α-galactosidase A expression cassette comprises SEQ ID NO:16. In one embodiment, the nucleotide sequence encoding the human α-galactosidase A expression cassette is set forth in SEQ ID NO:16. In specific embodiments, the nucleotide sequence encoding the human α-galactosidase A expression cassette comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:17. In one embodiment, the nucleotide sequence encoding the human α-galactosidase A expression cassette comprises SEQ ID NO:17. In one embodiment, the nucleotide sequence encoding the human α-galactosidase A expression cassette is set forth in SEQ ID NO:17. In specific embodiments, the nucleotide sequence encoding the human α-galactosidase A expression cassette comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:18. In one embodiment, the nucleotide sequence encoding the human α-galactosidase A expression cassette comprises SEQ ID NO:18. In one embodiment, the nucleotide sequence encoding the human α-galactosidase A expression cassette is set forth in SEQ ID NO:18. In specific embodiments, the nucleotide sequence encoding the human α-galactosidase A expression cassette comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:19. In one embodiment, the nucleotide sequence encoding the human α-galactosidase A expression cassette comprises SEQ ID NO:19. In one embodiment, the nucleotide sequence encoding the human α-galactosidase A expression cassette is set forth in SEQ ID NO:19. Without wishing to be bound by any one theory, these nucleotide sequences are particularly suitable for gene therapies for Fabry disease.

The present disclosure provides, inter alia, polynucleotides, promoters, and expression cassettes related to these nucleotide sequences.

### 5.2.1 Polynucleotides

In one aspect, provided herein is a polynucleotide comprising a nucleotide sequence encoding human α-galactosidase A, wherein the nucleotide sequence encoding human α-galactosidase A comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:1, SEQ ID NO:2, or SEQ ID NO:20.

In certain embodiments, the nucleotide sequence encoding human α-galactosidase A comprises SEQ ID NO:1. In certain embodiments, the nucleotide sequence encoding human α-galactosidase A is set forth in SEQ ID NO:1.

In certain embodiments, the nucleotide sequence encoding human α-galactosidase A comprises SEQ ID NO:2. In certain embodiments, the nucleotide sequence encoding human α-galactosidase A is set forth in SEQ ID NO:2.

In certain embodiments, the nucleotide sequence encoding human α-galactosidase A comprises SEQ ID NO:20. In certain embodiments, the nucleotide sequence encoding human α-galactosidase A is set forth in SEQ ID NO:20.

In one aspect, provided herein is a polynucleotide comprising a nucleotide sequence encoding a promoter, wherein the nucleotide sequence encoding the promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In specific embodiments, the promoter is a hepatocyte-specific promoter. In specific embodiments, the promoter is a hepatocyte-myocyte bispecific promoter. In specific embodiments, the promoter is a hepatocyte-kidney cell bispecific promoter. In specific embodiments, the promoter is a hepatocyte-myocyte-kidney cell multi-specific promoter.

In certain embodiments, the nucleotide sequence encoding the promoter comprises SEQ ID NO:3. In certain embodiments, the nucleotide sequence encoding the promoter is set forth in SEQ ID NO:3.

In certain embodiments, the nucleotide sequence encoding the promoter comprises SEQ ID NO:4. In certain embodiments, the nucleotide sequence encoding the promoter is set forth in SEQ ID NO:4.

In certain embodiments, the nucleotide sequence encoding the promoter comprises SEQ ID NO:5. In certain embodiments, the nucleotide sequence encoding the promoter is set forth in SEQ ID NO:5.

In certain embodiments, the nucleotide sequence encoding the promoter comprises SEQ ID NO:6. In certain embodiments, the nucleotide sequence encoding the promoter is set forth in SEQ ID NO:6.

In certain embodiments, the nucleotide sequence encoding the promoter comprises SEQ ID NO:7. In certain embodiments, the nucleotide sequence encoding the promoter is set forth in SEQ ID NO:7.

In certain embodiments, the nucleotide sequence encoding the promoter comprises SEQ ID NO:8. In certain embodiments, the nucleotide sequence encoding the promoter is set forth in SEQ ID NO:8.

In certain embodiments, the nucleotide sequence encoding the promoter comprises SEQ ID NO:9. In certain embodiments, the nucleotide sequence encoding the promoter is set forth in SEQ ID NO:9.

In certain embodiments, the nucleotide sequence encoding the promoter comprises SEQ ID NO:10. In certain embodiments, the nucleotide sequence encoding the promoter is set forth in SEQ ID NO:10.

In one aspect, provided herein is a polynucleotide comprising a nucleotide sequence encoding human α-galactosidase A and a nucleotide sequence encoding a promoter that is operably linked to the nucleotide sequence encoding human α-galactosidase A, wherein the nucleotide sequence encoding human α-galactosidase A comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:1, SEQ ID NO:2, or SEQ ID NO:20.

In certain embodiments, the nucleotide sequence encoding human α-galactosidase A comprises SEQ ID NO:1. In certain embodiments, the nucleotide sequence encoding human α-galactosidase A is set forth in SEQ ID NO:1.

In certain embodiments, the nucleotide sequence encoding human α-galactosidase A comprises SEQ ID NO:2. In certain embodiments, the nucleotide sequence encoding human α-galactosidase A is set forth in SEQ ID NO:2.

In certain embodiments, the nucleotide sequence encoding human α-galactosidase A comprises SEQ ID NO:20. In certain embodiments, the nucleotide sequence encoding human α-galactosidase A is set forth in SEQ ID NO:20.

In specific embodiments, the promoter is a hepatocyte-specific promoter. In specific embodiments, the promoter is a hepatocyte-myocyte bispecific promoter. In specific embodiments, the promoter is a hepatocyte-kidney cell bispecific promoter. In specific embodiments, the promoter is a hepatocyte-myocyte-kidney cell multi-specific promoter.

In various embodiments, the nucleotide sequence encoding the promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In certain embodiments, the nucleotide sequence encoding the promoter comprises SEQ ID NO:3. In certain embodiments, the nucleotide sequence encoding the promoter is set forth in SEQ ID NO:3.

In certain embodiments, the nucleotide sequence encoding the promoter comprises SEQ ID NO:4. In certain embodiments, the nucleotide sequence encoding the promoter is set forth in SEQ ID NO:4.

In certain embodiments, the nucleotide sequence encoding the promoter comprises SEQ ID NO:5. In certain embodiments, the nucleotide sequence encoding the promoter is set forth in SEQ ID NO:5.

In certain embodiments, the nucleotide sequence encoding the promoter comprises SEQ ID NO:6. In certain embodiments, the nucleotide sequence encoding the promoter is set forth in SEQ ID NO:6.

In certain embodiments, the nucleotide sequence encoding the promoter comprises SEQ ID NO:7. In certain embodiments, the nucleotide sequence encoding the promoter is set forth in SEQ ID NO:7.

In certain embodiments, the nucleotide sequence encoding the promoter comprises SEQ ID NO:8. In certain embodiments, the nucleotide sequence encoding the promoter is set forth in SEQ ID NO:8.

In certain embodiments, the nucleotide sequence encoding the promoter comprises SEQ ID NO:9. In certain embodiments, the nucleotide sequence encoding the promoter is set forth in SEQ ID NO:9.

In certain embodiments, the nucleotide sequence encoding the promoter comprises SEQ ID NO:10. In certain embodiments, the nucleotide sequence encoding the promoter is set forth in SEQ ID NO:10.

In various embodiments, the polynucleotide further comprises one or more regulatory control elements (see Section 5.2.5). In a specific embodiment, the polynucleotide further comprises a nucleotide sequence encoding a polyA signal.

In one aspect, provided herein is a polynucleotide comprising a nucleotide sequence encoding a biologic molecule and a nucleotide sequence encoding a promoter that is operably linked to the nucleotide sequence encoding the biologic molecule, wherein the nucleotide sequence encoding the promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In specific embodiments, the promoter is a hepatocyte-specific promoter. In specific embodiments, the promoter is a hepatocyte-myocyte bispecific promoter. In specific embodiments, the promoter is a hepatocyte-kidney cell bispecific promoter. In specific embodiments, the promoter is a hepatocyte-myocyte-kidney cell multi-specific promoter.

In certain embodiments, the nucleotide sequence encoding the promoter comprises SEQ ID NO:3. In certain embodiments, the nucleotide sequence encoding the promoter is set forth in SEQ ID NO:3.

In certain embodiments, the nucleotide sequence encoding the promoter comprises SEQ ID NO:4. In certain embodiments, the nucleotide sequence encoding the promoter is set forth in SEQ ID NO:4.

In certain embodiments, the nucleotide sequence encoding the promoter comprises SEQ ID NO:5. In certain embodiments, the nucleotide sequence encoding the promoter is set forth in SEQ ID NO:5.

In certain embodiments, the nucleotide sequence encoding the promoter comprises SEQ ID NO:6. In certain embodiments, the nucleotide sequence encoding the promoter is set forth in SEQ ID NO:6.

In certain embodiments, the nucleotide sequence encoding the promoter comprises SEQ ID NO:7. In certain embodiments, the nucleotide sequence encoding the promoter is set forth in SEQ ID NO:7.

In certain embodiments, the nucleotide sequence encoding the promoter comprises SEQ ID NO:8. In certain embodiments, the nucleotide sequence encoding the promoter is set forth in SEQ ID NO:8.

In certain embodiments, the nucleotide sequence encoding the promoter comprises SEQ ID NO:9. In certain embodiments, the nucleotide sequence encoding the promoter is set forth in SEQ ID NO:9.

In certain embodiments, the nucleotide sequence encoding the promoter comprises SEQ ID NO:10. In certain embodiments, the nucleotide sequence encoding the promoter is set forth in SEQ ID NO:10.

More descriptions of biologic molecules are provided in Section 5.2.4. In a specific embodiment, the biologic molecule is human α-galactosidase A (preferably a functional human α-galactosidase A).

In various embodiments, the polynucleotide further comprises one or more regulatory control elements (see Section 5.2.5). In a specific embodiment, the polynucleotide further comprises a nucleotide sequence encoding a polyA signal.

In one aspect, provided herein is a polynucleotide comprising a nucleotide sequence encoding an expression cassette, wherein the nucleotide sequence encoding the expression cassette comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:16, SEQ ID NO:17, SEQ ID NO:18, or SEQ ID NO:19.

In certain embodiments, the nucleotide sequence encoding the expression cassette comprises SEQ ID NO:16. In certain embodiments, the nucleotide sequence encoding the expression cassette is set forth in SEQ ID NO:16.

In certain embodiments, the nucleotide sequence encoding the expression cassette comprises SEQ ID NO:17. In certain embodiments, the nucleotide sequence encoding the expression cassette is set forth in SEQ ID NO:17.

In certain embodiments, the nucleotide sequence encoding the expression cassette comprises SEQ ID NO:18. In certain embodiments, the nucleotide sequence encoding the expression cassette is set forth in SEQ ID NO:18.

In certain embodiments, the nucleotide sequence encoding the expression cassette comprises SEQ ID NO:19. In certain embodiments, the nucleotide sequence encoding the expression cassette is set forth in SEQ ID NO:19.

In one aspect, provided herein is a polynucleotide comprising a nucleotide sequence encoding an expression cassette described in Section 5.2.3.

In various aspects and embodiments, the polynucleotide described herein is purified. In various aspects and embodiments, the polynucleotide described herein is isolated. In various aspects and embodiments, the polynucleotide described herein is a recombinant polynucleotide.

In certain embodiments, the polynucleotide described herein is a DNA polynucleotide. In a specific embodiment, the polynucleotide described herein is a double-stranded DNA. In a specific embodiment, the polynucleotide described herein is a single-stranded DNA.

More descriptions of polynucleotides are provided in Section 5.1

### 5.2.2 Promoters

In one aspect, provided herein is a promoter, which is encoded by a nucleotide sequence comprising a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In specific embodiments, the promoter is a hepatocyte-specific promoter. In specific embodiments, the promoter is a hepatocyte-myocyte bispecific promoter. In specific embodiments, the promoter is a hepatocyte-kidney cell bispecific promoter. In specific embodiments, the promoter is a hepatocyte-myocyte-kidney cell multi-specific promoter.

In certain embodiments, the nucleotide sequence encoding the promoter comprises SEQ ID NO:3. In certain embodiments, the nucleotide sequence encoding the promoter is set forth in SEQ ID NO:3.

In certain embodiments, the nucleotide sequence encoding the promoter comprises SEQ ID NO:4. In certain embodiments, the nucleotide sequence encoding the promoter is set forth in SEQ ID NO:4.

In certain embodiments, the nucleotide sequence encoding the promoter comprises SEQ ID NO:5. In certain embodiments, the nucleotide sequence encoding the promoter is set forth in SEQ ID NO:5.

In certain embodiments, the nucleotide sequence encoding the promoter comprises SEQ ID NO:6. In certain embodiments, the nucleotide sequence encoding the promoter is set forth in SEQ ID NO:6.

In certain embodiments, the nucleotide sequence encoding the promoter comprises SEQ ID NO:7. In certain embodiments, the nucleotide sequence encoding the promoter is set forth in SEQ ID NO:7.

In certain embodiments, the nucleotide sequence encoding the promoter comprises SEQ ID NO:8. In certain embodiments, the nucleotide sequence encoding the promoter is set forth in SEQ ID NO:8.

In certain embodiments, the nucleotide sequence encoding the promoter comprises SEQ ID NO:9. In certain embodiments, the nucleotide sequence encoding the promoter is set forth in SEQ ID NO:9.

In certain embodiments, the nucleotide sequence encoding the promoter comprises SEQ ID NO:10. In certain embodiments, the nucleotide sequence encoding the promoter is set forth in SEQ ID NO:10.

In various aspects and embodiments, the promoter described herein is a recombinant promoter.

More descriptions of promoters are provided in Section 5.1 and Section 5.2.5.

### 5.2.3 Expression Cassettes

In one aspect, provided herein is an expression cassette comprising a nucleotide sequence encoding human α-galactosidase A and a nucleotide sequence encoding a promoter that is operably linked to the nucleotide sequence encoding human α-galactosidase A, wherein the nucleotide sequence encoding human α-galactosidase A comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:1, SEQ ID NO:2, or SEQ ID NO:20.

In certain embodiments, the nucleotide sequence encoding human α-galactosidase A comprises SEQ ID NO:1. In certain embodiments, the nucleotide sequence encoding human α-galactosidase A is set forth in SEQ ID NO:1.

In certain embodiments, the nucleotide sequence encoding human α-galactosidase A comprises SEQ ID NO:2. In certain embodiments, the nucleotide sequence encoding human α-galactosidase A is set forth in SEQ ID NO:2.

In certain embodiments, the nucleotide sequence encoding human α-galactosidase A comprises SEQ ID NO:20. In certain embodiments, the nucleotide sequence encoding human α-galactosidase A is set forth in SEQ ID NO:20.

In specific embodiments, the promoter is a hepatocyte-specific promoter. In specific embodiments, the promoter is a hepatocyte-myocyte bispecific promoter. In specific embodiments, the promoter is a hepatocyte-kidney cell bispecific promoter. In specific embodiments, the promoter is a hepatocyte-myocyte-kidney cell multi-specific promoter.

In various embodiments, the nucleotide sequence encoding the promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In certain embodiments, the nucleotide sequence encoding the promoter comprises SEQ ID NO:3. In certain embodiments, the nucleotide sequence encoding the promoter is set forth in SEQ ID NO:3.

In certain embodiments, the nucleotide sequence encoding the promoter comprises SEQ ID NO:4. In certain embodiments, the nucleotide sequence encoding the promoter is set forth in SEQ ID NO:4.

In certain embodiments, the nucleotide sequence encoding the promoter comprises SEQ ID NO:5. In certain embodiments, the nucleotide sequence encoding the promoter is set forth in SEQ ID NO:5.

In certain embodiments, the nucleotide sequence encoding the promoter comprises SEQ ID NO:6. In certain embodiments, the nucleotide sequence encoding the promoter is set forth in SEQ ID NO:6.

In certain embodiments, the nucleotide sequence encoding the promoter comprises SEQ ID NO:7. In certain embodiments, the nucleotide sequence encoding the promoter is set forth in SEQ ID NO:7.

In certain embodiments, the nucleotide sequence encoding the promoter comprises SEQ ID NO:8. In certain embodiments, the nucleotide sequence encoding the promoter is set forth in SEQ ID NO:8.

In certain embodiments, the nucleotide sequence encoding the promoter comprises SEQ ID NO:9. In certain embodiments, the nucleotide sequence encoding the promoter is set forth in SEQ ID NO:9.

In certain embodiments, the nucleotide sequence encoding the promoter comprises SEQ ID NO:10. In certain embodiments, the nucleotide sequence encoding the promoter is set forth in SEQ ID NO:10.

In various embodiments, the expression cassette further comprises one or more regulatory control elements (see Section 5.2.5). In a specific embodiment, the expression cassette further comprises a nucleotide sequence encoding a polyA signal.

In one aspect, provided herein is an expression cassette comprising a nucleotide sequence encoding a biologic molecule and a nucleotide sequence encoding a promoter that is operably linked to the nucleotide sequence encoding the biologic molecule, wherein the nucleotide sequence encoding the promoter comprises a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In specific embodiments, the promoter is a hepatocyte-specific promoter. In specific embodiments, the promoter is a hepatocyte-myocyte bispecific promoter. In specific embodiments, the promoter is a hepatocyte-kidney cell bispecific promoter. In specific embodiments, the promoter is a hepatocyte-myocyte-kidney cell multi-specific promoter.

In certain embodiments, the nucleotide sequence encoding the promoter comprises SEQ ID NO:3. In certain embodiments, the nucleotide sequence encoding the promoter is set forth in SEQ ID NO:3.

In certain embodiments, the nucleotide sequence encoding the promoter comprises SEQ ID NO:4. In certain embodiments, the nucleotide sequence encoding the promoter is set forth in SEQ ID NO:4.

In certain embodiments, the nucleotide sequence encoding the promoter comprises SEQ ID NO:5. In certain embodiments, the nucleotide sequence encoding the promoter is set forth in SEQ ID NO:5.

In certain embodiments, the nucleotide sequence encoding the promoter comprises SEQ ID NO:6. In certain embodiments, the nucleotide sequence encoding the promoter is set forth in SEQ ID NO:6.

In certain embodiments, the nucleotide sequence encoding the promoter comprises SEQ ID NO:7. In certain embodiments, the nucleotide sequence encoding the promoter is set forth in SEQ ID NO:7.

In certain embodiments, the nucleotide sequence encoding the promoter comprises SEQ ID NO:8. In certain embodiments, the nucleotide sequence encoding the promoter is set forth in SEQ ID NO:8.

In certain embodiments, the nucleotide sequence encoding the promoter comprises SEQ ID NO:9. In certain embodiments, the nucleotide sequence encoding the promoter is set forth in SEQ ID NO:9.

In certain embodiments, the nucleotide sequence encoding the promoter comprises SEQ ID NO:10. In certain embodiments, the nucleotide sequence encoding the promoter is set forth in SEQ ID NO:10.

More descriptions of biologic molecules are provided in Section 5.2.4. In a specific embodiment, the biologic molecule is human α-galactosidase A (preferably a functional human α-galactosidase A).

In various embodiments, the expression cassette further comprises one or more regulatory control elements (see Section 5.2.5). In a specific embodiment, the expression cassette further comprises a nucleotide sequence encoding a polyA signal.

In one aspect, provided herein is an expression cassette which is encoded by a nucleotide sequence comprising a nucleotide sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, or 99.8% or 100% identical to SEQ ID NO:16, SEQ ID NO:17, SEQ ID NO:18, or SEQ ID NO:19.

In certain embodiments, the nucleotide sequence encoding the expression cassette comprises SEQ ID NO:16. In certain embodiments, the nucleotide sequence encoding the expression cassette is set forth in SEQ ID NO:16.

In certain embodiments, the nucleotide sequence encoding the expression cassette comprises SEQ ID NO:17. In certain embodiments, the nucleotide sequence encoding the expression cassette is set forth in SEQ ID NO:17.

In certain embodiments, the nucleotide sequence encoding the expression cassette comprises SEQ ID NO:18. In certain embodiments, the nucleotide sequence encoding the expression cassette is set forth in SEQ ID NO:18.

In certain embodiments, the nucleotide sequence encoding the expression cassette comprises SEQ ID NO:19. In certain embodiments, the nucleotide sequence encoding the expression cassette is set forth in SEQ ID NO:19.

In various aspects and embodiments, the expression cassette described herein is a recombinant expression cassette.

More descriptions of expression cassettes are provided in Section 5.1.

### 5.2.4 Biologic Molecules

The biologic molecule described in this disclosure can be, for example but not limited to, a polypeptide, a protein, a nucleic acid (e.g., DNA or RNA), or an oligonucleotide (e.g., siRNA, shRNA, miRNA or aptamer). In specific embodiments, the biologic molecule is a polypeptide or a protein. In preferred embodiments, the biologic molecule is a human polypeptide or a human protein. The biologic molecule can be a reporter molecule, such as a reporter protein (for example, a fluorescent protein (e.g., green fluorescent protein (GFP)), luciferase (e.g., firefly luciferase), β-lactamase, or β-galactosidase (LacZ)). The biologic molecule can also be a therapeutic molecule, such as a therapeutic protein. A therapeutic molecule may be used to correct or ameliorate gene deficiencies associated with a disease or disorder. Exemplary therapeutic molecules may include, without limitation, enzymes, cytokines, growth factors, kinases, dominant negative mutant proteins, antibodies and antigen-binding fragments thereof, interleukins, hormones, and differentiation factors, .

In certain embodiments, the biologic molecule is a therapeutic protein. Therapeutic proteins described herein include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor alpha superfamily, including TGFalpha, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other therapeutic proteins described herein include those that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors alpha and beta, interferons alpha, beta, and gamma, stem cell factor, flk-2/fit3 ligand. Proteins produced by the immune system are also useful. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful proteins also include complement regulatory proteins such as complement regulatory proteins, membrane cofactor protein (MCP), decay accelerating factor (DAF), CR1, CF2 and CD59.

Other therapeutic proteins described herein are receptors for the hormones, growth factors, cytokines, lymphokines, regulatory proteins and immune system proteins. The disclosure encompasses receptors for cholesterol regulation and/or lipid modulation, including the low-density lipoprotein (LDL) receptor, high density lipoprotein (HDL) receptor, the very low density lipoprotein (VLDL) receptor, and scavenger receptors. The disclosure also encompasses proteins such as members of the steroid hormone receptor superfamily including glucocorticoid receptors and estrogen receptors, Vitamin D receptors and other nuclear receptors. In addition, useful proteins include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZFS, NFAT, CREB, HNF-4, C/EBP, SPI, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful biologic molecules described herein include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence. Still other useful proteins include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding beta-glucuronidase (GUSB)).

Other useful biologic molecules described herein also include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Other suitable biologic molecules described herein include those which may be useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

The biologic molecule may be used to reduce and/or modulate expression of a protein of interest for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene ber/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

In certain embodiments, the biologic molecule is RNA. These RNA molecules include but are not limited to molecules that exert their function through RNA interference (shRNA, RNAi), micro-RNA regulation (miR), catalytic RNA, antisense RNA, RNA aptamers, etc.

The biologic molecule may be used to induce an immune response to a selected antigen. For example, in order to promote an immune response, the antigen may be expressed from a promoter disclosed herein, the vector can be adjuvanted as described herein, and/or the vector can be put into degenerating tissue.

Examples of suitable immunogenic antigens include those selected from a variety of viral families. Example of desirable viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picomavirus family of viruses, target antigens include the VP1, VP2, VP3, VP4, and VPG. Other viral families include the astroviruses and the calcivirus family. The calcivirus family encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinatin encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis, and which include the putative cause of sudden acute respiratory syndrome (SARS). Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the arterivirus family and the rhabdovirus family. The rhabdovirus family includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus may be a suitable source of antigens. The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. Another source of antigens is the bornavirus family. The reovirus family includes the genera reovirus, rotavirus (which causes acute gastroenteritis in children), orbiviruses, and cultivirus (Colorado Tick fever, Lebombo (humans), equine encephalosis, blue tongue). The retrovirus family includes the sub-family oncorivirinal which encompasses such human and veterinary diseases as feline leukemia virus, HTLVI and HTLVII, lentivirinal (which includes HIV, simian immunodeficiency virus, feline immunodeficiency virus, equine infectious anemia virus, and spumavirinal). The papovavirus family includes the sub-family polyomaviruses (BKU and JCU viruses) and the sub-family papillomavirus (associated with cancers or malignant progression of papilloma). The adenovirus family includes viruses (EX, AD7, ARD, O. B. ) which cause respiratory disease and/or enteritis. The parvovirus family feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (HCMV, muromegalovirus) and the sub-family gamma herpesvirinae, which includes the genera lymphocryptovirus, EBV (Burkitts lymphoma), human herpesviruses 6A, 6B and 7, Kaposi's sarcoma-associated herpesvirus and cercopithecine herpesvirus (B virus), infectious rhinotracheitis, Marek's disease virus, and rhadinovirus. The poxvirus family includes the sub-family chordopoxvirinae, which encompasses the genera orthopoxvirus (Variola major (Smallpox) and Vaccinia (Cowpox)), parapoxvirus, avipoxvirus, capripoxvirus, leporipoxvirus, suipoxvirus, and the sub-family entomopoxvirinae. The hepadnavirus family includes the Hepatitis B virus. One unclassified virus which may be suitable source of antigens is the Hepatitis delta virus, Hepatitis E virus, and prions. Another virus which is a source of antigens is Nipan Virus. Still other viral sources may include avian infectious bursal disease virus and porcine respiratory and reproductive syndrome virus. The alphavirus family includes equine arteritis virus and various Encephalitis viruses.

The present disclosure may also encompass protein based immunogens as biologic molecules which are useful to immunize a human or non-human animal against other pathogens including bacteria, fungi, parasitic microorganisms or multicellular parasites which infect human and non-human vertebrates, or from a cancer cell or tumor cell. Examples of bacterial pathogens include pathogenic gram-positive cocci include pneumococci; staphylococci (and the toxins produced thereby, e.g., enterotoxin B); and streptococci. Pathogenic gram-negative cocci include meningococcus; gonococcus. Pathogenic enteric gram-negative bacilli include enterobacteriaceae; pseudomonas, acinetobacteria and eikenella; melioidosis; salmonella; shigella; haemophilus; moraxella; H. ducreyi (which causes chancroid); brucella species (brucellosis); Francisella tularensis (which causes tularemia); Yersinia pestis (plague) and other Yersinia (pasteurella); streptobacillus moniliformis and spirillum; Gram-positive bacilli include Listeria monocytogenes; erysipelothrix rhusiopathiae; Corynebacterium diphtheria (diphtheria); cholera; B. anthracia (anthrax); donovanosis (granuloma inguinale); and bartonellosis. Diseases caused by pathogenic anaerobic bacteria include tetanus; botulism (Clostridum botulinum and its toxin); Clostridium perfringens and its epsilon toxin; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include glanders (Burkholderia mallei); actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever (Coxiella burnetti), and Rickettsialpox. Examples of mycoplasma and chlamydial infections include: Mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis; trypanosomiasis; toxoplasmosis; Pneumocystis carinii; Trichans; Toxoplasma gondii; babesiosis; giardiasis; trichinosis; filariasis; schistosomiasis; nematodes; trematodes or flukes; and cestode (tapeworm) infections.

Many of these organisms and/or the toxins produced thereby have been identified by the Centers for Disease Control [(CDC), Department of Health and Human Services, USA], as agents which have potential for use in biological attacks. For example, some of these biological agents, include, Bacillus anthracia (anthrax), Clostridium botulinum and its toxin (botulism), Yersinia pestis (plague), variola major (smallpox), Francisella tularensis (tularemia), and viral hemorrhagic fevers [filoviruses (e.g., Ebola, Marburg], and arenaviruses [e.g., Lassa, Machupo]), all of which are currently classified as Category A agents; Coxiella burnetti (Q fever); Brucella species (brucellosis), Burkholderia mallei (glanders), Burkholderia pseudomallei (meloidosis), Ricinus communis and its toxin (ricin toxin), Clostridium perfringens and its toxin (epsilon toxin), Staphylococcus species and their toxins (enterotoxin B), Chlamydia psittaci (psittacosis), water safety threats (e.g., Vibrio cholerae, Crytosporidium parvum), Typhus fever (Richettsia powazekii), and viral encephalitis (alphaviruses, e.g., Venezuelan equine encephalitis; eastern equine encephalitis; western equine encephalitis); all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to deliver antigens from these organisms, viruses, their toxins or other by-products, which will prevent and/or treat infection or other adverse reactions with these biological agents.

In certain embodiments, the biologic molecule is a protein that is a segment of a variable region of T cells eliciting an immune response, i.e., to eliminate cytotoxic T cells. In rheumatoid arthritis (RA), several specific variable regions of TCRs which are involved in the disease have been characterized. These TCRs include V-3, V-14, V-17 and V-17. Thus, delivery of a nucleic acid sequence that encodes at least one of these polypeptides will elicit an immune response that will target T cells involved in RA. In multiple sclerosis (MS), several specific variable regions of TCRs which are involved in the disease have been characterized. These TCRs include V-7 and V-10. Thus, delivery of a transgene that encodes at least one of these polypeptides will elicit an immune response that will target T cells involved in MS. In scleroderma, several specific variable regions of TCRs which are involved in the disease have been characterized. These TCRs include V-6, V-8, V-14 and V-16, V-3C, V-7, V-14, V-15, V-16, V-28 and V-12. Thus, delivery of a nucleic acid molecule that encodes at least one of these polypeptides will elicit an immune response that will target T cells involved in scleroderma.

In specific embodiments, the biologic molecule is normally expressed in hepatocytes (e.g., normally expressed at a higher level in hepatocytes relative to other cell types). In specific embodiments, the biologic molecule is normally expressed in myocytes (e.g., normally expressed at a higher level in myocytes relative to other cell types). In specific embodiments, the biologic molecule is normally expressed in kidney cells (e.g., normally expressed at a higher level in kidney cells relative to other cell types). In specific embodiments, the biologic molecule is normally expressed in both hepatocytes and myocytes (e.g., expressed at higher level in both hepatocytes and myocytes relative to other cell types). In specific embodiments, the biologic molecule is normally expressed in both hepatocytes and kidney cells (e.g., expressed at higher level in both hepatocytes and kidney cells relative to other cell types). In specific embodiments, the biologic molecule is normally expressed in hepatocytes, myocytes, and kidney cells (e.g., expressed at higher level in hepatocytes, myocytes, and kidney cells relative to other cell types). In specific embodiments, the biologic molecule functions in hepatocytes (e.g., is required or important for the normal function of hepatocytes). In specific embodiments, the biologic molecule functions in myocytes (e.g., is required or important for the normal function of myocytes). In specific embodiments, the biologic molecule functions in kidney cells (e.g., is required or important for the normal function of kidney cells). In specific embodiments, the biologic molecule functions in both hepatocytes and myocytes (e.g., is required or important for the normal function of both hepatocytes and myocytes). In specific embodiments, the biologic molecule functions in both hepatocytes and kidney cells (e.g., is required or important for the normal function of both hepatocytes and kidney cells). In specific embodiments, the biologic molecule functions in hepatocytes, myocytes, and kidney cells (e.g., is required or important for the normal function of hepatocytes, myocytes, and kidney cells). In specific embodiments, the myocytes described herein are skeletal muscle cells (e.g., biceps, triceps, quadriceps, tibialis anterior, gastroenemius, rectus abdominis, diaphragm and/or pectoralis major cells) and/or heart muscle cells (e.g., heart atrium muscle and/or heart ventricle muscle cells). In particular embodiments, muscle can function as a production factory for a biologic molecule, which is secreted from muscle cells and eventually taken up by the cells of another tissue or organ to exert its therapeutic effect.

In specific embodiments, the biologic molecule described herein is associated with a lysosomal storage disorder. In specific embodiments, the deficiency in a biologic molecule described herein is at least a partial cause for a lysosomal storage disorder. In specific embodiments, the abnormal low or lack of expression of a biologic molecule described herein is at least a partial cause for a lysosomal storage disorder.

In a specific embodiment, the biologic molecule is a protein that is α-galactosidase A (GLA) (preferably human GLA). In a specific embodiment, the biologic molecule is a protein that is Scavenger Receptor Class B Member 2 (SCARB2) (preferably human SCARB2). In a specific embodiment, the biologic molecule is a protein that is aspartylglucosaminidase (AGA) (preferably human AGA). In a specific embodiment, the biologic molecule is a protein that is palmitoyl-protein thioesterase 1 (PPT1) (preferably human PPTI). In a specific embodiment, the biologic molecule is a protein that is tripeptidyl peptidase 1 (TPP1) (preferably human TPP1). In a specific embodiment, the biologic molecule is a protein that is CLN3 lysosomal/endosomal transmembrane protein, battenin (CLN3) (preferably human CLN3). In a specific embodiment, the biologic molecule is a protein that is DnaJ heat shock protein family (Hsp40) member C5 (DNAJC5) (preferably human DNAJC5). In a specific embodiment, the biologic molecule is a protein that is CLN5 intracellular trafficking protein (CLN5) (preferably human CLN5). In a specific embodiment, the biologic molecule is a protein that is CLN6 transmembrane ER protein (CLN6) (preferably human CLN6). In a specific embodiment, the biologic molecule is a protein that is Major Facilitator Superfamily Domain Containing 8 (MFSD8) (preferably human MFSD8). In a specific embodiment, the biologic molecule is a protein that is CLN8 transmembrane ER and ERGIC protein (CLN8) (preferably human CLN8). In a specific embodiment, the biologic molecule is a protein that is cathepsin D (CTSD) (preferably human CTSD). In a specific embodiment, the biologic molecule is a protein that is granulin precursor (GRN) (preferably human GRN). In a specific embodiment, the biologic molecule is a protein that is ATPase cation transporting 13A2 (ATP13A2) (preferably human ATP13A2). In a specific embodiment, the biologic molecule is a protein that is cathepsin F (CTSF) (preferably human CTSF). In a specific embodiment, the biologic molecule is a protein that is Potassium Channel Tetramerization Domain Containing 7 (KCTD7) (preferably human KCTD7). In a specific embodiment, the biologic molecule is a protein that is cystinosin, lysosomal cystine transporter (CTNS) (preferably human CTNS). In a specific embodiment, the biologic molecule is a protein that is lysosomal associated membrane protein 2 (LAMP2) (preferably human LAMP2). In a specific embodiment, the biologic molecule is a protein that is N-acylsphingosine amidohydrolase 1 (ASAH1) (preferably human ASAH1). In a specific embodiment, the biologic molecule is a protein that is alpha-L-fucosidase 1 (FUCA1) (preferably human FUCAI). In a specific embodiment, the biologic molecule is a protein that is cathepsin A (CTSA) (preferably human CTSA). In a specific embodiment, the biologic molecule is a protein that is beta-glucocerebrosidase (GBA) (preferably human GBA). In a specific embodiment, the biologic molecule is a protein that is acid alpha-glucosidase (GAA) (preferably human GAA). In a specific embodiment, the biologic molecule is a protein that is myosin VA (MYO5A) (preferably human MYO5A). In a specific embodiment, the biologic molecule is a protein that is RAB27A, Member RAS Oncogene Family (RAB27A) (preferably human RAB27A). In a specific embodiment, the biologic molecule is a protein that is Lysosomal Trafficking Regulator (LYST) (preferably human LYST). In a specific embodiment, the biologic molecule is a protein that is galactosidase beta 1 (GLB1) (preferably human GLB1). In a specific embodiment, the biologic molecule is a protein that is hexosaminidase subunit alpha (HEXA) (preferably human HEXA). In a specific embodiment, the biologic molecule is a protein that is hexosaminidase subunit beta (HEXB) (preferably human HEXB). In a specific embodiment, the biologic molecule is a protein that is ganglioside GM2 activator (GM2A) (preferably human GM2A). In a specific embodiment, the biologic molecule is a protein that is HPS1 biogenesis of lysosomal organelles complex 3 subunit 1 (HPS1) (preferably human HPS1). In a specific embodiment, the biologic molecule is a protein that is adaptor related protein complex 3 subunit beta 1 (AP3B1 or HPS2) (preferably human HPS2). In a specific embodiment, the biologic molecule is a protein that is HPS3 biogenesis of lysosomal organelles complex 2 subunit 1 (HPS3) (preferably human HPS3). In a specific embodiment, the biologic molecule is a protein that is HPS4 biogenesis of lysosomal organelles complex 3 subunit 2 (HPS4) (preferably human HPS4). In a specific embodiment, the biologic molecule is a protein that is HPS5 biogenesis of lysosomal organelles complex 2 subunit 2 (HPS5) (preferably human HPS5). In a specific embodiment, the biologic molecule is a protein that is HPS6 biogenesis of lysosomal organelles complex 2 subunit 3 (HPS6) (preferably human HPS6). In a specific embodiment, the biologic molecule is a protein that is dystrobrevin binding protein 1 (DTNBP1 or HPS7) (preferably human HPS7). In a specific embodiment, the biologic molecule is a protein that is biogenesis of lysosomal organelles complex 1 subunit 3 (BLOC1S3 or HPS8) (preferably human HPS8). In a specific embodiment, the biologic molecule is a protein that is biogenesis of lysosomal organelles complex 1 subunit 6 (BLOC1 S6 or HPS9) (preferably human HPS9). In a specific embodiment, the biologic molecule is a protein that is galactosylceramidase (GALC) (preferably human GALC). In a specific embodiment, the biologic molecule is a protein that is mannosidase alpha class 2B member 1 (MAN2B1) (preferably human MAN2B1). In a specific embodiment, the biologic molecule is a protein that is mannosidase beta (MANBA) (preferably human MANBA). In a specific embodiment, the biologic molecule is a protein that is arylsulfatase A (ARSA) (preferably human ARSA). In a specific embodiment, the biologic molecule is a protein that is N-acetylglucosamine-1-phosphate transferase subunits alpha and beta (GNPTAB) (preferably human GNPTAB). In a specific embodiment, the biologic molecule is a protein that is N-acetylglucosamine-1-phosphate transferase subunits alpha and beta (GNPTAB) (preferably human GNPTAB). In a specific embodiment, the biologic molecule is a protein that is N-acetylglucosamine-1-phosphate transferase subunit gamma (GNPTG) (preferably human GNPTG). In a specific embodiment, the biologic molecule is a protein that is mucolipin TRP cation channel 1 (MCOLN1) (preferably human MCOLN1). In a specific embodiment, the biologic molecule is a protein that is alpha-L-iduronidase (IDUA) (preferably human IDUA). In a specific embodiment, the biologic molecule is a protein that is iduronate 2-sulfatase (IDS) (preferably human IDS). In a specific embodiment, the biologic molecule is a protein that is N-sulfoglucosamine sulfohydrolase (SGSH) (preferably human SGSH). In a specific embodiment, the biologic molecule is a protein that is N-acetyl-alpha-glucosaminidase (NAGLU) (preferably human NAGLU). In a specific embodiment, the biologic molecule is a protein that is heparan-alpha-glucosaminide N-acetyltransferase (HGSNAT) (preferably human HGSNAT). In a specific embodiment, the biologic molecule is a protein that is glucosamine (N-acetyl)-6-sulfatase (GNS) (preferably human GNS). In a specific embodiment, the biologic molecule is a protein that is galactosamine (N-acetyl)-6-sulfatase (GALNS) (preferably human GALNS). In a specific embodiment, the biologic molecule is a protein that is galactosidase beta 1 (GLB1) (preferably human GLB1). In a specific embodiment, the biologic molecule is a protein that is arylsulfatase B (ARSB) (preferably human ARSB). In a specific embodiment, the biologic molecule is a protein that is beta-glucuronidase (GUSB) (preferably human GUSB). In a specific embodiment, the biologic molecule is a protein that is hyaluronidase 1 (HYAL1) (preferably human HYAL1). In a specific embodiment, the biologic molecule is a protein that is Sulfatase Modifying Factor I (SUMF1) (preferably human SUMF1). In a specific embodiment, the biologic molecule is a protein that is sphingomyelin phosphodiesterase 1 (SMPD1) (preferably human SMPD1). In a specific embodiment, the biologic molecule is a protein that is NPC Intracellular Cholesterol Transporter 1 (NPC1) (preferably human NPC1). In a specific embodiment, the biologic molecule is a protein that is NPC Intracellular Cholesterol Transporter 2 (NPC2) (preferably human NPC2). In a specific embodiment, the biologic molecule is a protein that is Solute Carrier Family 17 Member 5 (SLC17A5) (preferably human SLC17A5). In a specific embodiment, the biologic molecule is a protein that is alpha-N-acetylgalactosaminidase (NAGA) (preferably human NAGA). In a specific embodiment, the biologic molecule is a protein that is neuraminidase 1 (NEU1) (preferably human NEU1). In a specific embodiment, the biologic molecule is a protein that is lipase A, lysosomal acid type (LIPA) (preferably human LIPA). In a specific embodiment, the biologic molecule is a protein that is a Survival Motor Neuron (SMN) protein (preferably human SMN protein). In a specific embodiment, the biologic molecule is a protein that is microdystrophin (preferably human microdystrophin). In a specific embodiment, the biologic molecule is a protein that is phenylalanine hydroxylase (PAH) (preferably human PAH). In a specific embodiment, the biologic molecule is a protein that is Coagulation Factor VIII (FVIII) (preferably human FVIII). In a specific embodiment, the biologic molecule is a protein that is Coagulation Factor IX (FIX) (preferably human FIX). In preferred embodiments, the biologic molecule is a functional protein (e.g., a wild-type protein).

### 5.2.5 Regulatory Control Elements

Regulatory control elements include both expression control elements that are contiguous with the biologic molecule-encoding nucleotide sequence of interest and expression control elements that act in trans or act in cis but at a distance to control the expression of the biologic molecule of interest.

Expression control elements include, without limitation, appropriate transcription initiation, termination, promoter and enhancer sequences; polyadenylation (polyA) signals; sequences that stabilize cytoplasmic mRNA; sequences that enhance translation efficiency (i.e., Kozak consensus sequence); sequences that enhance protein stability; secretion signals; and nuclear localization sequences.

In various embodiments, the regulatory control element is a promoter. In certain embodiments, the promoter is a constitutive promoter. In certain embodiments, the promoter is an inducible promoter. In certain embodiments, the promoter is a native promoter of the biologic molecule-encoding nucleotide sequence. In certain embodiments, the promoter is a tissue-specific promoter. In preferred embodiments, the tissue-specific promoter is a hepatocyte (i.e., liver)-specific promoter. In a specific embodiment, the tissue-specific promoter is a hepatocyte (i.e., liver)-myocyte (i.e., muscle) bispecific promoter. In a specific embodiment, the tissue-specific promoter is a hepatocyte (i.e., liver)-kidney bispecific promoter. In a specific embodiment, the tissue-specific promoter is a hepatocyte (i.e., liver)-myocyte (i.e., muscle)-kidney multi-specific promoter. The muscle can be heart muscle (e.g., heart atrium muscle, and/or heart ventricle muscle) and/or skeletal muscle (e.g., biceps, triceps, quadriceps, tibialis anterior, gastroenemius, rectus abdominis, diaphragm, pectoralis major).

More descriptions of regulatory control elements are provided in Section 5.1 and Section 5.3.

5.3 Vectors, Gene Delivery Systems, and Viral particles

The present disclosure also provides vectors and gene delivery systems related to the nucleotide sequences, polynucleotides, promoters, and/or expression cassettes described above.

In one aspect, provided herein is a vector comprising a polynucleotide described herein (e.g., a polynucleotide described in Section 5.2.1).

In one aspect, provided herein is a vector comprising a promoter described herein (e.g., a promoter described in Section 5.2.2).

In one aspect, provided herein is a vector comprising an expression cassette described herein (e.g., an expression cassette described in Section 5.2.3).

In various embodiments, a vector described herein is a plasmid. In various embodiments, a vector described herein is an expression vector.

In various embodiments, a vector described herein is a recombinant viral vector. In various embodiments, a vector described herein is a recombinant AAV vector.

More descriptions of vectors are provided below in this section and in Section 5.1.

Gene delivery systems provided herein include recombinant viral particles such as recombinant AAV particles and non-viral gene delivery systems.

A viral particle consists of a viral genome enclosed in a coat of protein referred to as a capsid. An “AAV particle” refers to an AAV virus composed of at least one AAV capsid protein and an encapsidated AAV genome. The AAV genome is a linear, single-stranded DNA molecule that contains inverted terminal repeats (ITRs) at the 5′ and 3′ termini of the viral genome. The ITRs function in cis as origins of DNA replication and as packaging signals for the viral genome.

In one aspect, provided herein is a recombinant viral particle comprising a polynucleotide described herein (e.g., a polynucleotide described in Section 5.2.1).

In one aspect, provided herein is a recombinant viral particle comprising a recombinant viral genome comprising an expression cassette described herein (e.g., an expression cassette described in Section 5.2.3).

In one aspect, provided herein is a recombinant viral particle comprising a vector described herein (e.g., a vector described in this section).

In one aspect, provided herein is a recombinant AAV particle comprising: (a) an AAV capsid; and (b) a recombinant AAV genome comprising an expression cassette described herein (e.g., an expression cassette described in Section 5.2.3) flanked by AAV-inverted terminal repeats (ITRs).

A number of viral based systems have been developed for gene transfer into mammalian cells. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Green and Sambrook, Molecular Cloning: A Laboratory Manual, 41^{th} Ed., Cold Spring Harbor Laboratory (Cold Spring Harbor, N.Y. 2012), and in other virology and molecular biology manuals.

In certain embodiments, the viral vector or viral particle provided herein is derived from an adenovirus. Exemplary vectors are based on or derived from HAd5, ChAd3, HAd26, HAd6, AdCH3NSmut, HAd35, ChAd63, HAd4, rcAd26. Recombinant adenovirus vectors can be constructed according to known methods in the art. See, e.g., O'Connor et al., Virology, 217(1):11-22 (1996); Hardy et al., Journal of Virology, 73(9):7835-7841 (1999); Hardy et al., Journal of Virology, 71(3):1842-1849 (1997). In some embodiments, third-generation adenoviral vectors (also called “high capacity adenoviral vectors” (HCAds), helper-dependent or “gutless” adenoviral vectors) can be used herein to deliver longer sequences. In some embodiments, the nucleic acid construct of interest is cloned into an adenoviral vector that only contains the ITRs and a packaging signal. A helper adenoviral vector may be co-transfected into HEK cells to generate the adenoviral particle. See Lee et al., Genes and Diseases, 4(2):43-63 (2007).

In certain embodiments, the viral vector or viral particle provided herein is derived from a lentivirus. Exemplary vectors are based on or derived from HIV-1, HIV-2, SIVSM, SIVAGM, EIAV, FIV, VNV, CAEV, or BIV. Lentiviral vectors can be produced according to the known methods in the art, e.g., as described in Cribbs et al., BMC Biotechnology, 13:98 (2003); Merten et al., Mol Ther Methods Clin Dev.,13 (3):16017 (2016); Durand and Cimarelli, Viruses, 3:132-159 (2011). In some embodiments, third-generation self-inactivating lentiviral vectors are used herein.

In certain embodiments, the viral vector or viral particle provided herein is derived from a herpes simplex virus (HSV). In some embodiments, the herpes simplex virus is a herpes simplex type 1 virus (HSV-1), a herpes simplex type 2 virus (HSV-2), or any derivative thereof. Exemplary vectors are based on or derived from HSV-1, HSV-2, CMV, VZV, EBV, and KSHV. HSV-based vectors can be constructed according the methods known in the art, e.g., as described in U.S. Pat. Nos. 7,078,029, 6,261,552, 5,998,174, 5,879,934, 5,849,572, 5,849,571, 5,837,532, 5,804,413, and 5,658,724, and International Patent Applications WO 91/02788, WO 96/04394, WO 98/15637, and WO 99/06583, which are incorporated herein by reference in their entireties.

In some embodiments, the HSV-based vector provided herein is an amplicon vector. In other embodiments, the HSV-based vector provided herein is a replication-defective vector. In yet other embodiments, the HSV-based vector provided herein is a replication-competent vector.

The amplicons are plasmid-derived vectors engineered to contain both the origin of HSV DNA replication (ori) and HSV cleavage-packaging recognition sequences (pac). When amplicons are transfected into mammalian cells with HSV helper functions, they are replicated, form head-to-tail linked concatamers and are then packaged into viral particles. There are two major methods currently used for producing amplicon particles, one based on infection with defective helper HSVs and the other based on transfection of HSV-1 genes, such as a set of pac-deleted overlapping cosmids or a pac-deleted and ICP27-deleted BAC-HSV-1. In some embodiments, amplicons used herein can accommodate large fragments of foreign DNA (e.g., up to 152 kb), including multiple copies of the transgene (e.g., up to 15 copies), and are non-toxic.

In some embodiments, an HSV-based vector used herein is deficient in at least one essential HSV gene, and the HSV-based vector may also comprise one or more deletions of non-essential genes. In some embodiments, the HSV-based vector is replication-deficient. Most replication-deficient HSV-based vectors contain a deletion to remove one or more intermediate-early, early, or late HSV genes to prevent replication. In other embodiments, the HSV-based vector is deficient in an immediate early gene selected from the group consisting of ICPO, ICP4, ICP22, ICP27, ICP47, and a combination thereof. In a specific embodiment, the HSV-based vector is deficient for all of ICPO. ICP4, ICP22, ICP27, and ICP47. Exemplary replication-competent vectors include NV-1020 (HSV-1), RAV9395 (HSV-2), AD-472 (HSV-2), NS-gEnull (HSV-1), and ImmunoVEX (HSV2). Exemplary replication-defective vectors include d15-29 (HSV-2), d15-29-41L (HSV-1), DISC-dH (HSV-1 and HSV-2), CJ9gD(HSV-1), TOH-OVA (HSV-1), d106 (HSV-1), d81(HSV-1), HSV-SIV d106(HSV-1), and d106 (HSV-1).

Replication-deficient HSV-based vectors are typically produced in complementing cell lines that provide gene functions not present in the replication-deficient HSV-based vectors, but required for viral propagation, at appropriate levels in order to generate high titers of viral vector stock. An exemplary cell line complements for at least one and, in some embodiments, all replication-essential gene functions not present in a replication-deficient HSV-based vector. For example, a HSV-based vector deficient in ICPO, ICP4, ICP22, ICP27, and ICP47 can be complemented by the human osteosarcoma line U2OS. The cell line can also complement non-essential genes that, when missing, reduce growth or replication efficiency (e.g., UL55). The complementing cell line can complement for a deficiency in at least one replication-essential gene function encoded by the early regions, immediate-early regions, late regions, viral packaging regions, virus-associated regions, or combinations thereof, including all HSV functions (e.g., to enable propagation of HSV amplicons, which comprise minimal HSV sequences, such as only inverted terminal repeats and the packaging signal or only ITRs and an HSV promoter). In some embodiments, the cell line is further characterized in that it contains the complementing genes in a non-overlapping fashion with the HSV-based vector, which minimizes, and practically eliminates, the possibility of the HSV-based vector genome recombining with the cellular DNA. Accordingly, the presence of replication competent HSV is minimized, if not avoided in the vector stock, which, therefore, is suitable for certain therapeutic purposes, especially gene therapy purposes. The construction of complementing cell lines involves standard molecular biology and cell culture techniques well known in the art.

In certain embodiments, the viral vector or viral particle provided herein is derived from an adeno-associated virus (AAV). More detailed descriptions related to AAV is provided in Sections 5.3.1 below.

The nucleotide sequences, promoters, expression cassettes described herein can be cloned into a viral vector using any known molecular cloning methods in the art, including, for example, using restriction endonuclease sites and one or more selectable markers.

In one embodiment, the viral vector genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety) such as an intron, or a synthetic intron with modified sequences from mammalian genomes. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps), and hybrid adenovirus splice donor/IgG splice acceptor (230 bps). In one embodiment, the intron may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260,270, 280, 290, 300, 310, 320, 330, 340, 350, 360, 370, 380, 390, 400, 410, 420, 430, 440, 450, 460, 470, 480, 490 or 500.

In some embodiments, the viral vector also contains a selectable marker gene or a reporter gene to select cells expressing the protein from the population of host cells transfected through viral vectors. Both selectable markers and reporter genes may be flanked by appropriate regulatory sequences to enable expression in the host cells. For example, the viral vector may contain transcription and translation terminators, initiation sequences, and promoters useful for regulation of the expression of the nucleic acid sequences.

Non-viral gene delivery systems provided herein include, without limitation, lipid-based gene delivery systems (e.g., liposome-based gene delivery systems), peptide-based gene delivery systems, inorganic gene delivery systems, and polymeric delivery systems. Non-limiting exemplary non-viral gene delivery systems are described in, e.g., Sung and Kim, 2019, Biomater Res 23:8; Zu and Gao, 2021, AAPS J 23:78; and Lan et al., 2022, Mol Cancer 21:71; each of which is incorporated herein in its entirety.

In one aspect, provided herein is a non-viral gene delivery system comprising a polynucleotide described herein (e.g., a polynucleotide described in Section 5.2.1).

In one aspect, provided herein is a non-viral gene delivery system comprising a polynucleotide that comprises a nucleotide sequence encoding an expression cassette described herein (e.g., an expression cassette described in Section 5.2.3).

In one aspect, provided herein is a non-viral gene delivery system comprising a vector described herein (e.g., a vector described in this section).

### 5.3.1 AAV Vectors and AAV Particles

Any AAV serotypes or variants thereof can be used in the present disclosure. AAV serotypes may include, but not limited to, AAVI (Genbank Accession No. NC_002077.1; HC000057.1), AAV2 (Genbank Accession No. NC_001401.2, JC527779.1), AAV2i8 (Asokan, A., 2010, Discov. Med. 9:399), AAV3 (Genbank Accession No. NC_001729.1), AAV3-B (Genbank Accession No. AF028705.1), AAV4 (Genbank Accession No. NC_001829.1), AAV5 (Genbank Accession No. NC_006152.1; JC527780.1), AAV6 (Genbank Accession No. AF028704.1; JC527781.1), AAV7 (Genbank Accession No. NC_006260.1; JC527782.1), AAV8 (Genbank Accession No. NC_006261.1; JC527783.1), AAV9 (Genbank Accession No AX753250.1; JC527784.1), AAV10 (Genbank Accession No AY631965.1), AAVrh10 (Genbank Accession No. AY243015.1), AAV11 (Genbank Accession No AY631966.1), AAV12 (Genbank Accession No DQ813647.1), AAV13 (Genbank Accession No EU285562.1), AAV LK03, AAVrh74, AAV DJ (Wu Z, et al., J Virol. 80:11393-7 (2006)), AAVAnc81, Anc82, Anc83, Anc84, Anc10, And113, Ancl26, or Anc127 (Zin, E. et al., Cell. Rep. 12:1056 (2016)), AAV_go.l (Arbetum, A. E. et al., J. Virol. 79:15238 (2005)), AAVhu.37, AAVrh8, AAVrh8R, and AAV rh.8 (Wang et al., Mol. Ther. 18:119-125 (2010), or variants thereof.

AAV variants include, but not limited to, AAV1 variants (e.g., AAV comprising AAV1 variant capsid proteins), AAV2 variants (e.g., AAV comprising AAV2 variant capsid proteins), AAV3 variants (e.g., AAV comprising AAV3 variant capsid proteins), AAV3-B variants (e.g., AAV comprising AAV3-B variant capsid proteins), AAV4 variants (e.g., AAV comprising AAV4 variant capsid proteins), AAV5 variants (e.g., AAV comprising AAV5 variant capsid proteins), AAV6 variants (e.g., AAV comprising AAV6 variant capsid proteins), AAV7 variants (e.g., AAV comprising AAV7 variant capsid proteins), AAV8 variants (e.g., AAV comprising AAV8 variant capsid proteins), AAVrh8, AAVrh8R (e.g., AAV comprising AAVrh8 or AAVrh8R variant capsid proteins), AAV9 variants (e.g., AAV comprising AAV9 variant capsid proteins), AAV10 variants (e.g., AAV comprising AAV10 variant capsid proteins), AAVrh0 variants (e.g., AAV comprising AAVrh10 variant capsid proteins), AAV11 variants (e.g., AAV comprising AAV11 variant capsid proteins), AAV12 variants (e.g., AAV comprising AAV12 variant capsid proteins), AAV13 variants (e.g., AAV comprising AAV13 variant capsid proteins), AAV LK03 variants (e.g., AAV comprising AAV LK03 variant capsid proteins), or AAVrh74 variants (e.g., AAV comprising AAVrh74 variant capsid proteins).

Recombinant AAV (rAAV) vectors used in the present disclosure can be constructed according to known techniques. In some embodiments, the rAAV vector is constructed to include operatively linked components in the direction of transcription. Control elements can include the recombinant promoters provided herein, response elements, a transcriptional initiation region, and a transcriptional termination region. The control elements can be selected based on the cell of interest. In some embodiments, the resulting rAAV vector construct comprising the operatively linked components is franked (5′ and 3′) with functional AAV ITR sequences. In some embodiments, the resulting r AAV vector can have a genome with a structure of: (5′AAV ITR)-(promoter)-(transgene of interest)-(3′AAV ITR).

In certain embodiments, a promoter described herein is operatively linked to at least one additional regulatory sequence. In certain embodiments, regulatory sequences may, for example, include enhancer sequences, e.g., upstream enhancer sequences (USEs), RNA processing signals, e.g., splicing signals, polyadenylation signal sequences, sequences that stabilize cytoplasmic mRNA, post-transcriptional regulatory elements (PREs) and/or microRNA (miRNA) target sequences. In certain embodiments, regulatory sequences may include sequences that enhance translation efficiency (e.g., Kozak sequences), sequences that enhance protein stability, and/or sequences that enhance protein processing and/or secretion. In some embodiments, the regulatory sequences included promote the transcription or expression of the transgene operably linked to a promoter described herein in the subject in vivo. The regulatory sequences can comprise control sequences normally associated with the transgene of interest or alternatively heterologous control sequences.

In certain embodiments, a regulatory sequence comprises a regulatory control element. In certain embodiments, the regulatory control element is 5′ of the transgene (that is, is present in 5′ untranslated regions; 5′ UTRs). In other embodiments, the regulatory control element is 3′ of the transgene (that is, is present in 3′ untranslated regions; 3′ UTRs). In certain embodiments, the regulatory sequence comprises more than one regulatory control element, for example may comprise two, three, four or five control elements. In instances wherein the regulatory sequence comprises more than one control element, each control element may independently be 5′ of, 3′ of, flank, or within a promoter described herein.

In certain embodiments, the viral vector may comprise at least one polyadenylation (polyA) signal sequence, which are well known in the art. In instances where a polyadenylation sequence is present, it is generally located between the 3′ end of the transgene and the 5′ end of the 3′ ITR. In certain embodiments, the viral vector further comprises a polyA upstream enhancer sequence 5′ of the polyA signal sequence. In certain instances, the regulatory sequence is a sequence that increases translation efficiency, e.g., a Kozak sequence.

In certain embodiments, the viral vector comprises an intron. In certain embodiments, the intron is present within the transgene. In certain embodiments, the intron is 5′ or 3′ of the transgene. In certain embodiments, the intron flanks the 5′ or 3′ terminus of the transgene. In certain embodiments, the nucleic acid construct comprises two introns. In some embodiments, one intron is 5′ of and one intron is 3′ of the transgene. In certain embodiments, one intron flanks the 5′ terminus of the transgene and the second intron flanks the 3′ terminus of the transgene. In certain embodiments, the intron is an SV40 intron, e.g., a 5′ UTR SV40 intron.

The sequences of AAV ITR known in the art can be used in the present rAAV vector. In some embodiments, the AAV ITR used in the present vectors has a wild-type nucleotide sequence. In other embodiments, the AAV ITR sequence used in the present vectors is not wild-type sequence, and instead it comprises, e.g., the insertion, deletion or substitution of nucleotides. AAV ITRs provided herein may be derived from any AAV serotypes, including but not limited to, AAVI, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a variant thereof.

In some embodiments, the rAAV vector comprises an ITR from AAVI. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B. In some embodiments, the rAAV vector comprises an ITR from AAV4. In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12. In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some embodiments, the 5′ and 3′ ITRs which flank a nucleotide sequence in a rAAV vector provided herein are identical and derived from the same AAV serotype. In other embodiments, the 5′ and 3′ ITRs which flank a nucleotide sequence in a rAAV vector provided herein are different and/or derived from different AAV serotypes.

In some embodiments, the rAAV vector comprising the expression cassette of interest flanked by AAV ITRs can be constructed by directly inserting the cassette of interest into an AAV genome, e.g., into an excised AAV open reading frames, and certain portions of the AAV genome can optionally be deleted, as described in, e.g., WO 1993/003769; Kotin (1994) Human Gene Therapy 5:793-801; Shelling and Smith (1994) Gene Therapy 1:165-169; and Zhou et al. (1994) J. Exp. Med. 179:1867-1875.

In other embodiments, AAV ITRs are excised from an AAV genome or from an AAV vector containing such ITRs, and then are fused to 5′ and 3′ of the expression cassette of interest that is present in another vector using standard ligation techniques.

In certain embodiments, the rAAV vector provided herein comprises a recombinant self-complementing genome. A rAAV comprising a self-complementing genome can usually quickly form a double stranded DNA molecule by its partially complementing sequences (e.g., complementing coding and non-coding strands of a transgene). More specifically, in some embodiments, an rAAV vector provided herein comprises an rAAV genome that comprises a first heterologous polynucleotide sequence (e.g., a therapeutic transgene coding strand) and a second heterologous polynucleotide sequence (e.g., the noncoding or antisense strand of the therapeutic transgene), and the first heterologous polynucleotide sequence can form intrastrand base pairs with the second polynucleotide sequence. In some embodiments, the first heterologous polynucleotide sequence and a second heterologous polynucleotide sequence are linked by a sequence that facilitates intrastrand base-pairing, e.g., a hairpin DNA structure. In some embodiments, the first heterologous polynucleotide sequence and a second heterologous polynucleotide sequence are linked by a mutated ITR, so that the rep proteins do not cleave the viral genome at the mutated ITR. rAAV vectors comprising self-complementing genomes can be made using the methods known in the art, e.g., as described in U.S. Pat. Nos. 7,125,717; 7,785,888; 7,790,154; 7,846,729; 8,093,054; and 8,361,457.

In some embodiments, the recombinant viral genomes in the rAAV vectors provided herein is less than about 5 kilobases (kb) in size. In some embodiments, the recombinant viral genomes in the rAAV vectors provided herein is less than about 4.5 kb in size. In some embodiments, the recombinant viral genomes in the rAAV vectors provided herein is less than about 4.0 kb in size. In some embodiments, the recombinant viral genomes in the rAAV vectors provided herein is less than about 3.5 kb in size. In some embodiments, the recombinant viral genomes in the rAAV vectors provided herein is less than about 3.0 kb in size. In some embodiments, the recombinant viral genomes in the rAAV vectors provided herein is less than about 2.5 kb in size.

The capsid protein may be derived from the same serotype as the ITRs, or a derivative thereof. The capsid may also be of a different serotype than the ITR. For example, in certain embodiments, an AAV particle comprises AAV2 ITRs and an AAV6 capsid (AAV 2/6), AAV2 ITRs and an AAV7 capsid (AAV 2/7), AAV2 ITRs and an AAV8 capsid (AAV 2/8), or AAV2 ITRs and an AAV9 capsid (AAV 2/9).

Naturally occurring AAV capsids comprise AAV VP1, VP2 and VP3 capsid proteins, which are each encoded by splice variants of the AAV cap gene. In general, an AAV particle comprises three proteins, VP1, VP2 and VP3, with VP2 and VP3 being truncated version of VP1 so having sequences that are also comprised by VP1. Generally, the amino acid sequence of VP1 defines the serotype of the capsid. Thus, for example, if the VP1 capsid protein encodes for an AAV2 VP1 protein, AAV will be of the AAV2 serotype, whereas if the VP1 capsid protein encodes an AAV9 VP1 protein, the AAV will be of the AAV9 serotype.

Typically, AAV capsid proteins comprise a number of variable regions (VRs) and constant regions, which are located between the variable regions. The “GH loop” refers to a loop sequence flanked by β-strand G and β-strand H within the internal β-barrel of an AAV capsid protein, and comprises variable region VR IV through VR VIII.

The sequences of the VP2 and VP3 capsid proteins and the locations of various domains such as the variable regions and the GH loop can be readily and routinely determined by a skilled person from a given VP1 sequence by a suitable method known in the art, for example, by comparing or aligning the VP1 sequence with an annotated VP1 sequence (preferably, an annotated VP1 sequence from a closely related AAV species) using a suitable algorithm. Preferably, such an annotated VP1 sequence is an annotated wild-type AAV VP1 sequence.

In some embodiments, an AAV capsid protein (e.g., VP1, VP2 and/or VP3) in the present rAAV particle is not a naturally occurring capsid protein. In some embodiments, an AAV capsid protein (e.g., VP1, VP2 and/or VP3) is derived from a naturally occurring capsid protein.

In some embodiments, the AAV capsid protein is a VP1 capsid protein. In other embodiments, the AAV capsid protein is a VP2 capsid protein. In other embodiments, the AAV capsid protein is a VP3 capsid protein. In some embodiments, the rAAV particle comprises a VP1 capsid protein, a VP2 capsid protein and/or a VP3 capsid protein. In other embodiments, the rAAV particle comprises a VP1 capsid protein, a VP2 capsid protein and a VP3 capsid protein. In some embodiments, the rAAV particle comprises a VP1 capsid protein, a VP2 capsid protein and/or a VP3 capsid protein, wherein the capsid proteins of the rAAV particle are of the same serotype. In other embodiments, the rAAV particle comprises a VP1 capsid protein, a VP2 capsid protein and a VP3 capsid protein, wherein the capsid proteins of the AAV particle are of the same serotype.

In certain aspects, the capsid protein is a variant capsid protein. A variant capsid protein may comprise one or more mutations, e.g. amino acid substitutions, amino acid deletions, and heterologous peptide insertions, compared to a corresponding reference capsid protein such as the naturally occurring parental capsid protein, i.e. the capsid protein from which it was derived. In some embodiments the amino acid sequence of the AAV capsid protein is identical to the amino acid sequence of the wild type, or reference, or parent AAV capsid protein except for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues, e.g., except for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residue substitutions. In some embodiments, the capsid protein or AAV particle described herein may be a chimeric capsid protein or AAV particle, respectively, comprising a protein sequence of two or more AAV serotype capsid proteins or particles, respectively.

In various embodiments, a variant AAV capsid protein described herein is a variant AAV VP1 capsid protein. Also described herein are variant AAV capsid proteins that are the VP2 and VP3 counterparts of the VP1 capsid proteins described herein. Further described herein are fragments of such variant AAV capsid proteins (e.g., fragments of VP1, VP2 or VP3 capsid proteins), including, without limitation, variable regions (e.g., VR IV and VR VIII), GH loop and functional fragments that essentially retain the biological activity of the corresponding capsid proteins and their associated tissue tropism. In certain embodiments, fragments of a VP1, VP2 or VP3 capsid protein described herein are at least 7 amino acids in length. In certain embodiments, fragments of a VP1, VP2 or VP3 capsid protein described herein are at least 8 amino acids in length. In certain embodiments, fragments of a VP1, VP2 or VP3 capsid protein described herein are at least 9 amino acids in length. In certain embodiments, fragments of a VP1, VP2 or VP3 capsid protein described herein are at least 10 amino acids in length. In certain embodiments, fragments of a VP1, VP2 or VP3 capsid protein described herein are at least 20 amino acids in length. In certain embodiments, fragments of a VP1, VP2 or VP3 capsid protein described herein are at least 30 amino acids in length. In certain embodiments, fragments of a VP1, VP2 or VP3 capsid protein described herein are at least 40 amino acids in length. In certain embodiments, fragments of a VP1, VP2 or VP3 capsid protein described herein are at least 50 amino acids in length. In certain embodiments, fragments of a VP1, VP2 or VP3 capsid protein described herein are at least 100 amino acids in length. In certain embodiments, fragments of a VP1, VP2 or VP3 capsid protein described herein are at least 200 amino acids in length. In certain embodiments, fragments of a VP1, VP2 or VP3 capsid protein described herein are at least 300 amino acids in length. In certain embodiments, fragments of a VP1, VP2 or VP3 capsid protein described herein are at least 400 amino acids in length. In certain embodiments, fragments of a VP1, VP2 or VP3 capsid protein described herein are at least 500 amino acids in length. In certain embodiments, fragments of a VP1, VP2 or VP3 capsid protein described herein are 7-10 amino acids in length. In certain embodiments, fragments of a VP1, VP2 or VP3 capsid protein described herein are 10-50 amino acids in length. In certain embodiments, fragments of a VP1, VP2 or VP3 capsid protein described herein are 50-100 amino acids in length. In certain embodiments, fragments of a VP1, VP2 or VP3 capsid protein described herein are 100-200 amino acids in length. In certain embodiments, fragments of a VP1, VP2 or VP3 capsid protein described herein are 200-300 amino acids in length. In certain embodiments, fragments of a VP1, VP2 or VP3 capsid protein described herein are 300-400 amino acids in length. In certain embodiments, fragments of a VP1, VP2 or VP3 capsid protein described herein are 400-500 amino acids in length.

In some embodiments, the capsid protein in the rAAV particle provided herein is derived from an AAV1, AAV2, AAV2i8, AAV3, AAV3-B, AAV4, AAV5, AAV6, AAV7, AAV8, AAVrh8, AAVrh8R, AAV9, AAV10, AAVrhl0, AAV11, AAV12, AAV13, AAV-DJ, AAV LK03, AAVrh74, AAV44-9 capsid protein. In specific embodiments, the capsid protein in the rAAV particle provided herein has an amino acid sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% identical to the amino acid sequence of an AAV1, AAV2, AAV2i8, AAV3, AAV3-B, AAV4, AAV5, AAV6, AAV7, AAV8, AAVrh8, AAVrh8R, AAV9, AAV1O, AAVrh10, AAV11, AAV12, AAV13, AAV-DJ, AAV LK03, AAVrh74, AAV44-9 capsid protein.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV1.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV2.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV2i8.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV3.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV3-B.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV4.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV5.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV6.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV7.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV8.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrh8.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrh8R.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV9.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV10.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrhl0.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV111.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV12.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV13.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV-DJ.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV LK03.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrh74.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV44-9.

In some specific embodiments, the rAAV particle provided herein comprises an AAV VP1 capsid protein comprising an amino acid sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity to SEQ ID NO:21, SEQ ID NO:136, SEQ ID NO:137, SEQ ID NO:138, SEQ ID NO:139, SEQ ID NO:140 or SEQ ID NO:141, to its VP2 capsid protein counterpart, and/or to its VP3 capsid protein counterpart.

In some specific embodiments, the rAAV particle provided herein comprises an AAV VP1 capsid protein comprising an amino acid sequence of SEQ ID NO:21, SEQ ID NO:136, SEQ ID NO:137, SEQ ID NO:138, SEQ ID NO:139, SEQ ID NO:140 or SEQ ID NO:141, its VP2 capsid protein counterpart, and/or its VP3 capsid protein counterpart.

In specific embodiments, the rAAV particle provided herein is a recombinant AAV serotype 9 (rAAV9) particle. In specific embodiments, the rAAV particle provided herein comprises an AAV9 capsid protein. In particular embodiments, the AAV capsid of an rAAV particle provided herein comprises a variant AAV9 capsid protein.

In various embodiments, the variant AAV9 capsid protein comprises the amino acid sequence of SEQ ID NO:25, 27, 29, 31, 33, 35, 36, 38, 40, 42, 46, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, or 135.

In various embodiments, the variant AAV9 capsid protein comprises the amino acid sequence of SEQ ID NO:24, 26, 28, 30, 32, 34, 37, 39, 41, 43, 45, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112,114,116,118,120,122,124, 126, 128, 130, 132, or 134.

In various embodiments, the variant AAV9 capsid protein comprises the amino acid sequence of SEQ ID NO:31, 38, 49, 51, 53, 55, 57, 59 or 61.

In various embodiments, the variant AAV9 capsid protein comprises the amino acid sequence of SEQ ID NO:30, 37, 48, 50, 52, 54, 56, 58 or 60.

In certain embodiments, a variant AAV9 capsid protein provided herein comprises the amino acid sequence of SEQ ID NO:25. In specific embodiments, a variant AAV9 capsid protein provided herein comprises the amino acid sequence of SEQ ID NO: 24.

In certain embodiments, a variant AAV9 capsid protein provided herein comprises the amino acid sequence of SEQ ID NO:27. In specific embodiments, a variant AAV9 capsid protein provided herein comprises the amino acid sequence of SEQ ID NO:26.

In certain embodiments, a variant AAV9 capsid protein provided herein comprises the amino acid sequence of SEQ ID NO:29. In specific embodiments, a variant AAV9 capsid protein provided herein comprises the amino acid sequence of SEQ ID NO:28.

In certain embodiments, a variant AAV9 capsid protein provided herein comprises the amino acid sequence of SEQ ID NO:31. In specific embodiments, a variant AAV9 capsid protein provided herein comprises the amino acid sequence of SEQ ID NO:30.

In certain embodiments, a variant AAV9 capsid protein provided herein comprises the amino acid sequence of SEQ ID NO:33. In specific embodiments, a variant AAV9 capsid protein provided herein comprises the amino acid sequence of SEQ ID NO:32.

In certain embodiments, a variant AAV9 capsid protein provided herein comprises the amino acid sequence of SEQ ID NO:35. In certain embodiments, a variant AAV9 capsid protein provided herein comprises the amino acid sequence of SEQ ID NO: 36. In certain embodiments, a variant AAV9 capsid protein provided herein comprises the amino acid sequence of SEQ ID NO:35 and comprises the amino acid sequence of SEQ ID NO:36. In specific embodiments, a variant AAV9 capsid protein provided herein comprises the amino acid sequence of SEQ ID NO:34.

In certain embodiments, a variant AAV9 capsid protein provided herein comprises the amino acid sequence of SEQ ID NO:38. In specific embodiments, a variant AAV9 capsid protein provided herein comprises the amino acid sequence of SEQ ID NO:37.

In certain embodiments, a variant AAV9 capsid protein provided herein comprises the amino acid sequence of SEQ ID NO:40. In specific embodiments, a variant AAV9 capsid protein provided herein comprises the amino acid sequence of SEQ ID NO:39.

In certain embodiments, a variant AAV9 capsid protein provided herein comprises the amino acid sequence of SEQ ID NO:42. In specific embodiments, a variant AAV9 capsid protein provided herein comprises the amino acid sequence of SEQ ID NO:41.

In certain embodiments, a variant AAV9 capsid protein provided herein comprises the amino acid sequence of SEQ ID NO:44. In specific embodiments, a variant AAV9 capsid protein provided herein comprises the amino acid sequence of SEQ ID NO:43.

In certain embodiments, a variant AAV9 capsid protein provided herein comprises the amino acid sequence of SEQ ID NO:46. In certain embodiments, a variant AAV9 capsid protein provided herein comprises the amino acid sequence of SEQ ID NO:47. In certain embodiments, a variant AAV9 capsid protein provided herein comprises the amino acid sequence of SEQ ID NO:46 and comprises the amino acid sequence of SEQ ID NO:47. In specific embodiments, a variant AAV9 capsid protein provided herein comprises the amino acid sequence of SEQ ID NO:45.

In certain embodiments, a variant AAV9 capsid protein provided herein comprises the amino acid sequence of SEQ ID NO:49. In specific embodiments, a variant AAV9 capsid protein provided herein comprises the amino acid sequence of SEQ ID NO:48.

In certain embodiments, a variant AAV9 capsid protein provided herein comprises the amino acid sequence of SEQ ID NO:51. In specific embodiments, a variant AAV9 capsid protein provided herein comprises the amino acid sequence of SEQ ID NO:50.

In certain embodiments, a variant AAV9 capsid protein provided herein comprises the amino acid sequence of SEQ ID NO:53. In specific embodiments, a variant AAV9 capsid protein provided herein comprises the amino acid sequence of SEQ ID NO:52.

In certain embodiments, a variant AAV9 capsid protein provided herein comprises the amino acid sequence of SEQ ID NO:55. In specific embodiments, a variant AAV9 capsid protein provided herein comprises the amino acid sequence of SEQ ID NO:54.

In certain embodiments, a variant AAV9 capsid protein provided herein comprises the amino acid sequence of SEQ ID NO:57. In specific embodiments, a variant AAV9 capsid protein provided herein comprises the amino acid sequence of SEQ ID NO:56.

In certain embodiments, a variant AAV9 capsid protein provided herein comprises the amino acid sequence of SEQ ID NO:59. In specific embodiments, a variant AAV9 capsid protein provided herein comprises the amino acid sequence of SEQ ID NO:58.

In certain embodiments, a variant AAV9 capsid protein provided herein comprises the amino acid sequence of SEQ ID NO:61. In specific embodiments, a variant AAV9 capsid protein provided herein comprises the amino acid sequence of SEQ ID NO:60.

In certain embodiments, a variant AAV9 capsid protein provided herein comprises the amino acid sequence of SEQ ID NO:63. In specific embodiments, a variant AAV9 capsid protein provided herein comprises the amino acid sequence of SEQ ID NO:62.

In certain embodiments, a variant AAV9 capsid protein provided herein comprises the amino acid sequence of SEQ ID NO:65. In specific embodiments, a variant AAV9 capsid protein provided herein comprises the amino acid sequence of SEQ ID NO:64.

In certain embodiments, a variant AAV9 capsid protein provided herein comprises the amino acid sequence of SEQ ID NO:67. In specific embodiments, a variant AAV9 capsid protein provided herein comprises the amino acid sequence of SEQ ID NO:66.

In certain embodiments, a variant AAV9 capsid protein provided herein comprises the amino acid sequence of SEQ ID NO:69. In specific embodiments, a variant AAV9 capsid protein provided herein comprises the amino acid sequence of SEQ ID NO:68.

In certain embodiments, a variant AAV9 capsid protein provided herein comprises the amino acid sequence of SEQ ID NO:71. In specific embodiments, a variant AAV9 capsid protein provided herein comprises the amino acid sequence of SEQ ID NO:70.

In certain embodiments, a variant AAV9 capsid protein provided herein comprises the amino acid sequence of SEQ ID NO:73. In specific embodiments, a variant AAV9 capsid protein provided herein comprises the amino acid sequence of SEQ ID NO:72.

In certain embodiments, a variant AAV9 capsid protein provided herein comprises the amino acid sequence of SEQ ID NO:75. In specific embodiments, a variant AAV9 capsid protein provided herein comprises the amino acid sequence of SEQ ID NO:74.

In certain embodiments, a variant AAV9 capsid protein provided herein comprises the amino acid sequence of SEQ ID NO:77. In specific embodiments, a variant AAV9 capsid protein provided herein comprises the amino acid sequence of SEQ ID NO:76.

In certain embodiments, a variant AAV9 capsid protein provided herein comprises the amino acid sequence of SEQ ID NO:79. In specific embodiments, a variant AAV9 capsid protein provided herein comprises the amino acid sequence of SEQ ID NO:78.

In certain embodiments, a variant AAV9 capsid protein provided herein comprises the amino acid sequence of SEQ ID NO:81. In specific embodiments, a variant AAV9 capsid protein provided herein comprises the amino acid sequence of SEQ ID NO:80.

In certain embodiments, a variant AAV9 capsid protein provided herein comprises the amino acid sequence of SEQ ID NO:83. In specific embodiments, a variant AAV9 capsid protein provided herein comprises the amino acid sequence of SEQ ID NO:82.

In certain embodiments, a variant AAV9 capsid protein provided herein comprises the amino acid sequence of SEQ ID NO:85. In specific embodiments, a variant AAV9 capsid protein provided herein comprises the amino acid sequence of SEQ ID NO:84.

In certain embodiments, a variant AAV9 capsid protein provided herein comprises the amino acid sequence of SEQ ID NO:87. In specific embodiments, a variant AAV9 capsid protein provided herein comprises the amino acid sequence of SEQ ID NO:86.

In certain embodiments, a variant AAV9 capsid protein provided herein comprises the amino acid sequence of SEQ ID NO:89. In specific embodiments, a variant AAV9 capsid protein provided herein comprises the amino acid sequence of SEQ ID NO:88.

In certain embodiments, a variant AAV9 capsid protein provided herein comprises the amino acid sequence of SEQ ID NO:91. In specific embodiments, a variant AAV9 capsid protein provided herein comprises the amino acid sequence of SEQ ID NO:90.

In certain embodiments, a variant AAV9 capsid protein provided herein comprises the amino acid sequence of SEQ ID NO:93. In specific embodiments, a variant AAV9 capsid protein provided herein comprises the amino acid sequence of SEQ ID NO:92.

In certain embodiments, a variant AAV9 capsid protein provided herein comprises the amino acid sequence of SEQ ID NO:95. In specific embodiments, a variant AAV9 capsid protein provided herein comprises the amino acid sequence of SEQ ID NO:94.

In certain embodiments, a variant AAV9 capsid protein provided herein comprises the amino acid sequence of SEQ ID NO:97. In specific embodiments, a variant AAV9 capsid protein provided herein comprises the amino acid sequence of SEQ ID NO:96.

In certain embodiments, a variant AAV9 capsid protein provided herein comprises the amino acid sequence of SEQ ID NO:99. In specific embodiments, a variant AAV9 capsid protein provided herein comprises the amino acid sequence of SEQ ID NO:98.

In certain embodiments, a variant AAV9 capsid protein provided herein comprises the amino acid sequence of SEQ ID NO:101. In specific embodiments, a variant AAV9 capsid protein provided herein comprises the amino acid sequence of SEQ ID NO:100.

In certain embodiments, a variant AAV9 capsid protein provided herein comprises the amino acid sequence of SEQ ID NO:103. In specific embodiments, a variant AAV9 capsid protein provided herein comprises the amino acid sequence of SEQ ID NO:102.

In certain embodiments, a variant AAV9 capsid protein provided herein comprises the amino acid sequence of SEQ ID NO:105. In specific embodiments, a variant AAV9 capsid protein provided herein comprises the amino acid sequence of SEQ ID NO:104.

In certain embodiments, a variant AAV9 capsid protein provided herein comprises the amino acid sequence of SEQ ID NO:107. In specific embodiments, a variant AAV9 capsid protein provided herein comprises the amino acid sequence of SEQ ID NO:106.

In certain embodiments, a variant AAV9 capsid protein provided herein comprises the amino acid sequence of SEQ ID NO:109. In specific embodiments, a variant AAV9 capsid protein provided herein comprises the amino acid sequence of SEQ ID NO:108.

In certain embodiments, a variant AAV9 capsid protein provided herein comprises the amino acid sequence of SEQ ID NO:111. In specific embodiments, a variant AAV9 capsid protein provided herein comprises the amino acid sequence of SEQ ID NO:110.

In certain embodiments, a variant AAV9 capsid protein provided herein comprises the amino acid sequence of SEQ ID NO:113. In specific embodiments, a variant AAV9 capsid protein provided herein comprises the amino acid sequence of SEQ ID NO:112.

In certain embodiments, a variant AAV9 capsid protein provided herein comprises the amino acid sequence of SEQ ID NO:115. In specific embodiments, a variant AAV9 capsid protein provided herein comprises the amino acid sequence of SEQ ID NO:114.

In certain embodiments, a variant AAV9 capsid protein provided herein comprises the amino acid sequence of SEQ ID NO:117. In specific embodiments, a variant AAV9 capsid protein provided herein comprises the amino acid sequence of SEQ ID NO: 116.

In certain embodiments, a variant AAV9 capsid protein provided herein comprises the amino acid sequence of SEQ ID NO:119. In specific embodiments, a variant AAV9 capsid protein provided herein comprises the amino acid sequence of SEQ ID NO: 118.

In certain embodiments, a variant AAV9 capsid protein provided herein comprises the amino acid sequence of SEQ ID NO:121. In specific embodiments, a variant AAV9 capsid protein provided herein comprises the amino acid sequence of SEQ ID NO:120.

In certain embodiments, a variant AAV9 capsid protein provided herein comprises the amino acid sequence of SEQ ID NO:123. In specific embodiments, a variant AAV9 capsid protein provided herein comprises the amino acid sequence of SEQ ID NO:122.

In certain embodiments, a variant AAV9 capsid protein provided herein comprises the amino acid sequence of SEQ ID NO:125. In specific embodiments, a variant AAV9 capsid protein provided herein comprises the amino acid sequence of SEQ ID NO:124.

In certain embodiments, a variant AAV9 capsid protein provided herein comprises the amino acid sequence of SEQ ID NO:127. In specific embodiments, a variant AAV9 capsid protein provided herein comprises the amino acid sequence of SEQ ID NO:126.

In certain embodiments, a variant AAV9 capsid protein provided herein comprises the amino acid sequence of SEQ ID NO:129. In specific embodiments, a variant AAV9 capsid protein provided herein comprises the amino acid sequence of SEQ ID NO:128.

In certain embodiments, a variant AAV9 capsid protein provided herein comprises the amino acid sequence of SEQ ID NO:131. In specific embodiments, a variant AAV9 capsid protein provided herein comprises the amino acid sequence of SEQ ID NO:130.

In certain embodiments, a variant AAV9 capsid protein provided herein comprises the amino acid sequence of SEQ ID NO:133. In specific embodiments, a variant AAV9 capsid protein provided herein comprises the amino acid sequence of SEQ ID NO:132.

In certain embodiments, a variant AAV9 capsid protein provided herein comprises the amino acid sequence of SEQ ID NO:135. In specific embodiments, a variant AAV9 capsid protein provided herein comprises the amino acid sequence of SEQ ID NO:134.

In certain embodiments, a variant AAV9 capsid protein provided herein comprises the VP2 capsid protein counterpart of a VP1 capsid protein descried herein.

In certain embodiments, a variant AAV9 capsid protein provided herein comprises the VP3 capsid protein counterpart of a VP1 capsid protein descried herein.

In certain embodiments, a variant AAV9 capsid protein provided herein comprises a fragment (e.g., a functional fragment) of a VP1 capsid protein descried herein.

In various embodiments, a recombinant AAV particle described herein has an increased tropism for one or more tissues relative to a wild-type AAV particle. The one or more tissues can be, for example, liver, muscle (e.g., skeletal muscle (for example, biceps, triceps, quadriceps, tibialis anterior, gastroenemius, rectus abdominis, diaphragm and/or pectoralis major) and/or heart muscle (for example, heart atrium muscle and/or heart ventricle muscle)), kidney, brain (e.g., middle front brain, middle rear brain, front brain and/or rear brain), lung, and/or spleen.

In certain embodiments, a recombinant AAV particle described herein has an increased tropism for liver relative to a wild-type AAV particle.

In certain embodiments, a recombinant AAV particle described herein has an increased tropism for muscle (e.g., skeletal muscle (for example, biceps, triceps, quadriceps, tibialis anterior, gastroenemius, rectus abdominis, diaphragm and/or pectoralis major) and/or heart muscle (for example, heart atrium muscle and/or heart ventricle muscle)) relative to a wild-type AAV particle.

In certain embodiments, a recombinant AAV particle described herein has an increased tropism for kidney relative to a wild-type AAV particle.

In certain embodiments, a recombinant AAV particle described herein has an increased tropism for both liver and muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)) relative to a wild-type AAV particle.

In certain embodiments, a recombinant AAV particle described herein has an increased tropism for both liver and kidney relative to a wild-type AAV particle.

In certain embodiments, a recombinant AAV particle described herein has an increased tropism for liver, muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)) and kidney relative to a wild-type AAV particle.

In various embodiments, a recombinant AAV particle described herein has a decreased tropism for one or more tissues relative to a wild-type AAV particle. The one or more tissues can be, for example, muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)), kidney, brain (e.g., middle front brain, middle rear brain, front brain and/or rear brain), lung, and/or spleen.

In various embodiments, a recombinant AAV particle described herein has an increased tropism for one or more tissues as described above and a decreased tropism for another one or more tissues as described above.

In various embodiments, the tropism for a tissue described herein is measured after a subject is administered systemically with the recombinant AAV particle. In certain embodiments, the tropism for a tissue described herein is measured after a subject is administered intravenously with the recombinant AAV particle.

In preferred embodiments, a recombinant AAV particle described herein is a non-naturally occurring AAV particle. In certain embodiments, a recombinant AAV particle described herein is isolated. In certain embodiments, a recombinant AAV particle described herein is purified. 5.4 Cells

In one aspect, provided herein is a host cell comprising a polynucleotide described herein (e.g., a polynucleotide described in Section 5.2.1).

In one aspect, provided herein is a host cell comprising a vector described herein (e.g., a vector described in Section 5.3).

In certain embodiments, the host cell further comprises an AAV rep gene-encoding nucleotide sequence, in a polynucleotide or vector that is the same as or different from a polynucleotide or vector described herein (e.g., a polynucleotide described in Section 5.2.1 or a vector described in Section 5.3). In certain embodiments, the host cell further comprises an AAV cap gene-encoding nucleotide sequence, in a polynucleotide or vector that is the same as or different from a polynucleotide or vector described herein (e.g., a polynucleotide described in Section 5.2.1 or a vector described in Section 5.3). In certain embodiments, the host cell further comprises helper functions, such as one or more helper plasmid and/or one or more helper virus (see Section 5.5 regarding helper functions).

In one aspect, provided herein is a host cell comprising a recombinant viral particle described herein (e.g., a recombinant viral particle described in Section 5.3).

In aspect, provided herein is a host cell producing a recombinant viral particle described herein (e.g., a recombinant viral particle described in Section 5.3).

In one aspect, provided herein is a host cell comprising a recombinant AAV particle described herein (e.g., a recombinant AAV particle described in Section 5.3).

In aspect, provided herein is a host cell producing a recombinant AAV particle described herein (e.g., a recombinant AAV particle described in Section 5.3).

In one aspect, provided herein is a host cell comprising a non-viral delivery system described herein (e.g., a non-viral delivery system described in Section 5.3).

In aspect, provided herein is a host cell producing a non-viral delivery system described herein (e.g., a non-viral delivery system described in Section 5.3).

In some embodiments, the host cell is an ex vivo host cell. In some embodiments, the host cell is an in vitro host cell. In some embodiments, the host cell is an in vivo host cell.

As used herein, the term “host” refers to cells (e.g., cells from insects, animals (including humans and non-human animals), yeast, and bacteria, etc.) which harbor a polynucleotide, vector, viral particle (e.g., rAAV particle), or non-viral gene delivery system. It is not intended that the present disclosure be limited to any particular type of host cell. Indeed, it is contemplated that any suitable cell will find use herein as a host. A host cell may be in the form of or be derived from a single cell, a population, a culture (such as a liquid culture or a culture on a solid substrate), a cell line, an organism or part thereof.

The host cell may be used to expand, replicate, or produce a polynucleotide, vector, recombinant viral particle (e.g., rAAV particle), non-viral gene delivery system, or biologic molecule described herein. A host cell suitable for this purpose can be, for example, a bacterial cell, a yeast cell, an insect cell, or a mammalian cell (such as a human cell or a non-human mammalian cell). Non-limiting exemplary insect cells that can be used as host cells include Ao38, High Five, Sf9, Se301, SeIZD2109, SeUCRI, Sf900+, Sf21, BTI-TN-5B1-4, MG-1, Tn368, HzAml, BM-N, Ha2302 and Hz2E5. Non-limiting exemplary mammalian cells that can be used as host cells include HEK293, HEK293-T, HeLa, WEHI, 1OT1/2, MDCK, W138, Jurkat, 2V6.11, Saos, C2C12, L, HT1080, HepG2, COS1, BSC 1, BSC 40, BMT 10, CHO, NSO, SP2/0, PER.C6, Vero, RD, BHK, HT 1080, A549, Cos-7, ARPE-19, MRC-5, primary fibroblast, hepatocyte, and myoblast cells. In certain embodiments, the host cell is a human cell. In specific embodiments, the human cell is autologous to a subject (e.g., a human patient) to be treated. In specific embodiments, the human cell is allogeneic to a subject (e.g., a human patient) to be treated.

The host cell may also be used as a therapeutic per se. A host cell suitable for this purpose is preferably derived from the same species of the subject. Thus, if the subject to be treated is a human patient a host cell suitable for use as a therapeutic per se is preferably a human cell. In specific embodiments, the human cell is autologous to the subject (e.g., a human patient) to be treated by the human cell. In specific embodiments, the human cell is allogeneic to the subject (e.g., a human patient) to be treated by the human cell.

In one aspect, provided herein is a population of host cells stably transduced by a recombinant viral particle (e.g., recombinant AAV particle) described herein. In one aspect, provided herein is a population of host cells stably transduced by a non-viral gene delivery system described herein. In specific embodiments, the population of host cells are an ex vivo population of host cells. In specific embodiments, the population of host cells are an in vitro population of host cells. In specific embodiments, the population of host cells are an in vivo population of host cells. In specific embodiments, the population of host cells are a population of human host cells. In specific embodiments, the population of host cells are an ex vivo population of human host cells. In specific embodiments, the population of host cells are an in vitro population of human host cells.

In certain embodiments, such a population of host cells may be used to generate a population of recombinant viral particles (e.g., recombinant AAV particles) that are used as a therapeutic and administered to a subject (e.g., a human patient) in need thereof.

In certain embodiments, such a population of host cells may be used to generate a population of non-viral gene delivery system molecules that are used as a therapeutic and administered to a subject (e.g., a human patient) in need thereof.

In certain embodiments, such a population of host cells may be used to generate a population of biologic molecules as described in Section 5.2.4, which population of biologic molecules is used as a therapeutic and administered to a subject (e.g., a human patient) in need thereof.

In certain embodiments, such a population of host cells may be used as a therapeutic per se and be administered to a subject (e.g., a human patient) in need thereof. In specific embodiments, the population of host cells are autologous to the subject (e.g., a human patient) to be treated by the population of host cells. In specific embodiments, the population of host cells are allogeneic to the subject (e.g., a human patient) to be treated by the population of host cells.

### 5.5 Methods of Production

In another aspect, provided herein is a method of producing a recombinant viral particle described herein, such as a recombinant AAV particle described herein (e.g., a recombinant viral particle, such as a recombinant AAV particle, described in Section 5.3).

In another aspect, provided herein is a method of producing a non-viral gene delivery system described herein (e.g., a non-viral gene delivery system described in Section 5.3).

Also described herein are methods of producing a polynucleotide described herein (e.g., a polynucleotide described in Section 5.2.1), a promoter described herein (e.g., a promoter described in Section 5.2.2), an expression cassette described herein (e.g., an expression cassette described in Section 5.2.3), a vector described herein (e.g., a vector described in Section 5.3), or a host cell described herein (e.g., a host cell described in Section 5.4).

The polynucleotides, promoters, expression cassettes, vectors, host cells, recombinant viral particles (such as recombinant AAV particles), and non-viral gene delivery systems of the disclosure may be produced by any suitable means known in the art, including recombinant production, genetic engineering, molecular cloning, chemical synthesis, and other synthetic means. Such production methods are within the knowledge of those of skill in the art and are not a limitation of the present invention.

Generation of a polynucleotide, promoter, expression cassette, and/or vector of the disclosure may be made using any suitable genetic engineering and protein production techniques known in the art, including, without limitation, cloning, restriction endonuclease digestion, ligation, transformation, plasmid purification, DNA sequencing, chemical synthesis, in vitro translation and in vivo expression, for example as described in Green and Sambrook, Molecular Cloning: A Laboratory Manual, 4^{th} Ed., Cold Spring Harbor Laboratory (Cold Spring Harbor, N.Y. 2012).

A host cell may be made to carry a polynucleotide, vector, recombinant viral particle (such as a recombinant AAV particle), or non-viral gene delivery system of the disclosure using any suitable method, including, without limitation, transfection, electroporation, transduction, liposome delivery, membrane fusion techniques, high velocity DNA-coated pellets, viral infection and protoplast fusion.

Recombinant viral particles (such as recombinant AAV particles) may be produced by host cells that allow for the production and replication of the recombinant viral particles (such as recombinant AAV particles). Methods of producing recombinant viral particles including recombinant AAV particles are well known in the art and are described in, for example, Adeno-Associated Virus: Methods and Protocols (Methods In Molecular Biology, 280), ed. Snyder and Moullier, Humana Press, NJ (2011); Viral Vectors for Gene Therapy: Methods and Protocols (Methods in Molecular Biology, 1937); ed. Manfredsson and Benskey, Humana Press, NJ (2019); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); U.S. Pat. Nos. U.S. Pat. Nos. 5,064,764, 5,756,283, 6,194,191, 6,204,059, 6,258,595, 6,261,551, 6,270,996, 6,281,010, 6,365,394, 6,475,769, 6,482,634, 6,485,966, 6,566,118, 6,943,019, 6,953,690, 7,022,519, 7,238,526, 7,291,498, 7,491,508 and 8,137,948; and International Patent Application Publication Nos. WO1996039530, WO1998010088, WO1999014354, WO1999015685, WO1999047691, WO2000055342, WO2000075353, WO2001023597, WO2015191508, WO2018022608, WO2019217513, WO2019222132, WO2019222136 and WO2020232044; the disclosures of each of which are incorporated herein by reference in their entirety.

The rAAV particles described herein may be produced using any suitable method known in the art. For example, a host cell (e.g., a mammalian cell) may be engineered to stably express the necessary components for AAV particle production. This can be achieved by integrating a plasmid (or multiple plasmids) comprising AAV rep and cap genes, and a selectable marker, such as an antibiotic (e.g., neomycin or ampicillin) resistance gene into the genome of the cell. The cell can be, e.g., an insect or mammalian cell which can then be co-infected with a helper virus (e.g., adenovirus or baculovirus providing the helper functions) and the rAAV vector comprising the 5′ and 3′ AAV ITR. The use of a selectable marker allows for large-scale production of the rAAV. As another non-limiting example, adenovirus or baculovirus rather than plasmids can be used to introduce rep and cap genes into packaging cells. As yet another non-limiting example, both the viral vector containing the 5′ and 3′ AAV ITRs and the rep and cap genes can be stably integrated into the DNA of producer cells, and the helper functions can be provided by a wild-type adenovirus to produce the rAAV.

A helper virus for AAV refers to a virus that allows AAV to be replicated and packaged by a host cell. A helper virus provides helper functions that allow for the replication of AAV. A number of such helper viruses have been identified, including adenoviruses, herpesviruses and poxviruses such as vaccinia. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include EIA functions, EIB functions, E2A functions, VA functions and E4orf6 functions.

A preparation of AAV is said to be substantially free of helper virus if the ratio of infectious AAV particles to infectious helper virus particles is at least about 102:1; at least about 104:1, at least about 106:1; or at least about 108:1. Preparations can also be free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

In certain embodiments, host cells containing the rAAV vectors described above is rendered capable of providing AAV helper functions to replicate and encapsulate the transgene encoding the protein of interest flanked by the AAV ITRs to produce rAAV particles. AAV helper functions are generally AAV-derived coding sequences which can be expressed to provide AAV gene products that, in turn, function in trans for productive AAV replication. AAV helper functions are used herein to complement necessary AAV functions that are missing from the rAAV vectors. In some embodiments, AAV helper functions include one, or both of the major AAV ORFs, namely the rep and cap coding regions, or functional homologues thereof.

AAV helper functions can be introduced into the host cell by transfecting the host cell with an AAV helper construct either prior to, or concurrently with, the transfection of the rAAV vector. For example, AAV helper constructs can be used to provide at least transient expression of AAV rep and/or cap genes to complement missing AAV functions that are necessary for productive AAV infection. Typically, AAV helper constructs lack AAV ITRs and can neither replicate nor package themselves. The AAV helper constructs can be in the form of, e.g., a plasmid, phage, transposon, cosmid, virus, or virion.

In certain embodiments, the host cell is also capable of providing or is provided with non AAV-derived functions or “accessory functions” to produce rAAV particles. Accessory functions are non AAV-derived viral and/or cellular functions upon which AAV is dependent for its replication, such as non AAV proteins and RNAs that are required in AAV replication, including those involved in activation of AAV gene transcription, stage specific AAV mRNA splicing, AAV DNA replication, synthesis of Cap expression products and AAV capsid assembly. In some embodiments, viral-based accessory functions can be derived from a known helper virus.

In some embodiments, as a result of the infection of the host cell with a helper virus and/or an accessory function vector, a recombinant AAV particle is produced, and the produced rAAV particle is infectious, replication-defective virus, and includes an AAV protein shell that encapsulates a heterologous nucleotide sequence of interest flanked on both sides by AAV ITRs.

rAAV particles can be purified from the host cell using a purification method known in the art, such as chromatography, CsCl gradients, and other methods as described, for example, in U.S. Pat. Nos. 6,989,264 and 8,137,948 and WO 2010/148143. In some embodiments, residual helper virus can be inactivated using known methods, e.g., by heating.

### 5.6 Pharmaceutical Compositions

In one aspect, provided herein is a pharmaceutical composition comprising a recombinant viral particle described herein, such as a recombinant AAV particle described herein (e.g., a recombinant viral particle, such as a recombinant AAV particle described in Section 5.3) and a pharmaceutically acceptable carrier.

In one aspect, provided herein is a pharmaceutical composition comprising a non-viral gene delivery system described herein (e.g., a non-viral gene delivery system described in Section 5.3) and a pharmaceutically acceptable carrier.

In one aspect, provided herein is a pharmaceutical composition comprising a population of host cells described herein that are stably transduced by a recombinant viral particle described herein, such as a recombinant AAV particle described herein (e.g., a population of host cells stably transduced by a recombinant viral particle, such as a recombinant AAV particle, as described in Section 5.4) and a pharmaceutically acceptable carrier.

In one aspect, provided herein is a pharmaceutical composition comprising a population of host cells described herein that are stably transduced by a non-viral gene delivery system described herein (e.g., a population of host cells stably transduced by a non-viral gene delivery system, as described in Section 5.4) and a pharmaceutically acceptable carrier.

In certain embodiments, the concentration of a recombinant viral particle (e.g., a recombinant AAV particle) in a pharmaceutical composition described herein may range from 1×10⁸ vg/ml to 1×10²⁰ vg/ml. In specific embodiments, the concentration of a recombinant viral particle (e.g., a recombinant AAV particle) in a pharmaceutical composition described herein may range from 1×10⁹ vg/ml to 1×10′⁹ vg/ml. In specific embodiments, the concentration of a recombinant viral particle (e.g., a recombinant AAV particle) in a pharmaceutical composition described herein may range from 1×10¹¹ vg/ml to 1×10¹⁸ vg/ml. In specific embodiments, the concentration of a recombinant viral particle (e.g., a recombinant AAV particle) in a pharmaceutical composition described herein may range from 1×10¹⁰ vg/ml to 1×10¹⁷ vg/ml. In specific embodiments, the concentration of a recombinant viral particle (e.g., a recombinant AAV particle) in a pharmaceutical composition described herein may range from 1×10¹² vg/ml to 1×10¹⁶ vg/ml. In specific embodiments, the concentration of a recombinant viral particle (e.g., a recombinant AAV particle) in a pharmaceutical composition described herein may range from 1×10¹³ vg/ml to 1×10¹⁵ vg/ml. In certain embodiments, the concentration of a recombinant viral particle (e.g., a recombinant AAV particle) in a pharmaceutical composition described herein may range from 1×10⁸ vg/ml to 1×10¹⁵ vg/ml.

In certain embodiments, the concentration of host cells in a pharmaceutical composition described herein may range from 1×10² cells/ml to 1×10¹² cells/ml. In specific embodiments, the concentration of host cells in a pharmaceutical composition described herein may range from 1×10³ cells/ml to 1×10¹¹ cells/ml. In specific embodiments, the concentration of host cells in a pharmaceutical composition described herein may range from 1×10⁴ cells/ml to 1×10¹⁰ cells/ml. In specific embodiments, the concentration of host cells in a pharmaceutical composition described herein may range from 1×10⁵ cells/ml to 1×10⁹ cells/ml. In specific embodiments, the concentration of host cells in a pharmaceutical composition described herein may range from 1×10⁶ cells/ml to 1×10⁸ cells/ml.

In certain embodiments, the pharmaceutical composition described herein is injected in a volume between about 0.1 ml and about 20 ml. In certain embodiments, the pharmaceutical composition described herein is injected in a volume between about 1 ml and about 10 ml.

Typically, an agent (e.g., an excipient or carrier) is pharmaceutically acceptable when it is safe, non-toxic, and is not biologically or otherwise undesirable, and is acceptable for veterinary use as well as human pharmaceutical use.

In certain embodiments, a pharmaceutical composition described herein comprises one or more pharmaceutically acceptable excipients to provide the composition with advantageous properties for storage and/or administration to subjects for the treatment of a disease or disorder. In certain embodiments, a pharmaceutical composition described herein comprises one or more buffering agents, such as sodium phosphate dibasic and/or sodium phosphate monobasic monohydrate. In certain embodiments, a pharmaceutical composition described herein comprises one or more isotonicity agents, such as sodium chloride. In certain embodiments, a pharmaceutical composition described herein comprises one or more bulking agents, such as mannitol, sucrose, dextran, lactose, trehalose, and/or povidone (PVP K24). In certain embodiments, a pharmaceutical composition described herein comprises one or more surfactants, such as polysorbate 80, polysorbate 20, sodium dodecyl sulfate, sodium stearate, ammonium lauryl sulfate, TRITON AG 98 (Rhone-Poulenc), poloxamer 407, and/or poloxamer 188.

Preferably, the pharmaceutical compositions described herein are stable and can be stored for extended periods of time without an unacceptable change in quality, potency, or purity, for example, at below −60° C., at about −20° C., at about 2° C. to 8° C., and/or at room temperature.

Preferably, the pharmaceutical compositions described herein are sterile and stable under the conditions of manufacture and storage. Pharmaceutical compositions described herein may be formulated as a solution, microemulsion, liposome, lyophilized composition, or other ordered structure suitable to accommodate high drug concentration.

In certain embodiments, a pharmaceutical composition described herein is formulated for a route of administration to a subject. Non-limiting examples of routes of administration that can be used include direct delivery to the target organ, oral, inhalation, intravenous, intramuscular, subcutaneous, intradermal, intranasal, intrathecal, intrapancreatic, intraperitoneal, intratumoral, and other parental routes of administration. In specific embodiments, a pharmaceutical composition described herein is formulated for systemic administration to a subject. In a specific embodiment, a pharmaceutical composition described herein is formulated for intravenous administration to a subject.

In some embodiments, the pharmaceutical composition provided herein contains a viral particle described herein, a non-viral gene delivery system described herein, or a population of host cells described herein in amounts effective to treat or prevent a disease or disorder, such as a therapeutically effective or prophylactically effective amount. Therapeutic or prophylactic efficacy in some embodiments is monitored by periodic assessment of treated subjects.

### 5.7 Methods and Uses

In one aspect, provided herein is a method of treating a disease or disorder in a subject in need thereof, comprising administering to the subject, preferably a therapeutically effective amount of, a recombinant viral particle described herein, such as a recombinant AAV particle described herein (e.g., a recombinant viral particle, such as a recombinant AAV particle, described in Section 5.3).

In one aspect, provided herein is a method of treating a disease or disorder in a subject in need thereof, comprising administering to the subject, preferably a therapeutically effective amount of, a non-viral gene delivery system described herein (e.g., a non-viral gene delivery system described in Section 5.3).

In one aspect, provided herein is a method of treating a disease or disorder in a subject in need thereof, comprising administering to the subject, preferably a therapeutically effective amount of, a pharmaceutical composition described herein (e.g., a pharmaceutical composition described in Section 5.6).

In one aspect, provided herein is a method of delivering a biologic molecule to one or more ex vivo target cells, comprising transducing the one or more target cells with a recombinant viral particle described herein, such as a recombinant AAV particle described herein (e.g., a recombinant viral particle, such as a recombinant AAV particle, described in Section 5.3).

In one aspect, provided herein is a method of delivering a biologic molecule to one or more ex vivo target cells, comprising transducing the one or more target cells with a non-viral gene delivery system described herein (e.g., a non-viral gene delivery system described in Section 5.3).

In one aspect, provided herein is a method of delivering a biologic molecule to one or more in vitro target cells, comprising transducing the one or more target cells with a recombinant viral particle described herein, such as a recombinant AAV particle described herein (e.g., a recombinant viral particle, such as a recombinant AAV particle, described in Section 5.3).

In one aspect, provided herein is a method of delivering a biologic molecule to one or more in vitro target cells, comprising transducing the one or more target cells with a non-viral gene delivery system described herein (e.g., a non-viral gene delivery system described in Section 5.3).

In one aspect, provided herein is a method of delivering a biologic molecule to one or more in vivo target cells in a subject, comprising administering to the subject a recombinant viral particle described herein, such as a recombinant AAV particle described herein (e.g., a recombinant viral particle, such as a recombinant AAV particle, described in Section 5.3).

In one aspect, provided herein is a method of delivering a biologic molecule to one or more in vivo target cells in a subject, comprising administering to the subject a non-viral gene delivery system described herein (e.g., a non-viral gene delivery system described in Section 5.3).

In one aspect, provided herein is a method of delivering a biologic molecule to one or more in vivo target cells in a subject, comprising administering to the subject a pharmaceutical composition described herein (e.g., a pharmaceutical composition described in Section 5.6).

More description of biologic molecules are provided in Section 5.2.4.

In certain embodiments, the one or more target cells are one or more liver cells. In certain embodiments, the one or more target cells are one or more muscle cells. In certain embodiments, the one or more target cells are one or more kidney cells. In certain embodiments, the one or more target cells are one or more liver cells and one or more muscle cells. In certain embodiments, the one or more target cells are one or more liver cells and one or more kidney cells. In certain embodiments, the one or more target cells are one or more liver cells, one or more muscle cells, and one or more kidney cells. In specific embodiments, the one or more muscle cells described herein are one or more skeletal muscle cells (e.g., biceps, triceps, quadriceps, tibialis anterior, gastrocnemius, rectus abdominis, diaphragm and/or pectoralis major cell(s)) and/or one or more heart muscle cells (e.g., heart atrium muscle and/or heart ventricle muscle cell(s)). In particular embodiments, muscle can function as a production factory for a biologic molecule, which is secreted from muscle cells and eventually taken up by the cells of another tissue or organ to exert its therapeutic effect.

The route of administration or delivery and the amount of the recombinant viral particle (e.g., recombinant AAV particle), non-viral gene delivery system, or pharmaceutical composition to be administered to a subject can be determined based on the nature of the disease or disorder, condition of the subject and the knowledge of the physician. Non-limiting examples of routes of administration or delivery that can be used include direct delivery to the target organ, oral, inhalation, intravenous, intramuscular, subcutaneous, intradermal, intranasal, intrathecal, intrapancreatic, intraperitoneal, intratumoral, and other parental routes of administration. In a specific embodiment, a recombinant viral particle (e.g., recombinant AAV particle), non-viral gene delivery system, or a pharmaceutical composition described herein is administered or delivered systemically. In a specific embodiment, a recombinant viral particle (e.g., recombinant AAV particle), non-viral gene delivery system, or a pharmaceutical composition described herein is administered or delivered intravenously.

In certain embodiments, the method administers a recombinant viral particle (e.g., a recombinant AAV particle) described herein to a subject at a dose of 1×10⁸ to 1×10¹⁷ vector genomes (vg). In certain embodiments, the method administers a recombinant viral particle (e.g., a recombinant AAV particle) described herein to a subject at a dose of 1×10⁹ to 1×10¹⁶ vector genomes (vg). In certain embodiments, the method administers a recombinant viral particle (e.g., a recombinant AAV particle) described herein to a subject at a dose of 1×10¹⁰ to 1×10¹⁵ vector genomes (vg). In certain embodiments, the method administers a recombinant viral particle (e.g., a recombinant AAV particle) described herein to a subject at a dose of 1×10¹¹ to 1×10¹⁴ vector genomes (vg).

In certain embodiments, the method administers a recombinant viral particle (e.g., a recombinant AAV particle) described herein to a subject at a dose of 1×10⁸ to 1×10¹⁷ vector genomes/kg (vg/kg). In certain embodiments, the method administers a recombinant viral particle (e.g., a recombinant AAV particle) described herein to a subject at a dose of 1×10⁹ to 1×10¹⁶ vector genomes/kg (vg/kg). In certain embodiments, the method administers a recombinant viral particle (e.g., a recombinant AAV particle) described herein to a subject at a dose of 1×10¹⁰ to 1×10¹⁵ vector genomes/kg (vg/kg). In certain embodiments, the method administers a recombinant viral particle (e.g., a recombinant AAV particle) described herein to a subject at a dose of 1×10¹¹ to 1×10¹⁴ vector genomes/kg (vg/kg).

The methods of delivery and methods of treatment described herein can be used to treat various diseases or disorders, including, without limitation, genetic disorders (such as lysosomal storage disorders), cancers (such as carcinoma, sarcoma, leukemia, lymphoma, germ cell tumors, and blastoma), autoimmune diseases, and infectious diseases.

In specific embodiments, the disease or disorder is caused at least by dysfunction of the liver. In specific embodiments, the disease or disorder is caused at least by dysfunction of the muscle (e.g., skeletal muscle (e.g., biceps, triceps, quadriceps, tibialis anterior, gastroenemius, rectus abdominis, diaphragm and/or pectoralis major) and/or heart muscle (e.g., heart atrium muscle and/or heart ventricle muscle)). In specific embodiments, the disease or disorder is caused at least by dysfunction of the kidney. In specific embodiments, the disease or disorder is caused at least by dysfunction of the liver and dysfunction of the muscle (e.g., skeletal muscle (e.g., biceps, triceps, quadriceps, tibialis anterior, gastroenemius, rectus abdominis, diaphragm and/or pectoralis major). In specific embodiments, the disease or disorder is caused at least by dysfunction of the liver and dysfunction of the kidney. In specific embodiments, the disease or disorder is caused at least by dysfunction of the liver, dysfunction of the muscle (e.g., skeletal muscle (e.g., biceps, triceps, quadriceps, tibialis anterior, gastrocnemius, rectus abdominis, diaphragm and/or pectoralis major), and dysfunction of the kidney.

In specific embodiments, the disease or disorder is associated with the liver cells. In specific embodiments, the disease or disorder is associated with the muscle cells (e.g., skeletal muscle cells (e.g., biceps, triceps, quadriceps, tibialis anterior, gastrocnemius, rectus abdominis, diaphragm and/or pectoralis major cells) and/or heart muscle cells (e.g., heart atrium muscle and/or heart ventricle muscle cells)). In specific embodiments, the disease or disorder is associated with the kidney cells. In specific embodiments, the disease or disorder is associated with the liver cells and the muscle cells (e.g., skeletal muscle cells (e.g., biceps, triceps, quadriceps, tibialis anterior, gastrocnemius, rectus abdominis, diaphragm and/or pectoralis major cells) and/or heart muscle cells (e.g., heart atrium muscle and/or heart ventricle muscle cells)). In specific embodiments, the disease or disorder is associated with the liver cells and the kidney cells. In specific embodiments, the disease or disorder is associated with the liver cells, the muscle cells (e.g., skeletal muscle (e.g., biceps, triceps, quadriceps, tibialis anterior, gastrocnemius, rectus abdominis, diaphragm and/or pectoralis major cells) and/or heart muscle cells (e.g., heart atrium muscle and/or heart ventricle muscle cells)), and the kidney cells.

In certain embodiments, the disease or disorder is a lysosomal storage disorder.

In a specific embodiment, the disease or disorder is Fabry disease. In a specific embodiment, the disease or disorder is action myoclonus-renal failure syndrome. In a specific embodiment, the disease or disorder is Aspartylglucos-aminuria. In a specific embodiment, the disease or disorder is CLN1 disease or neuronal ceroid lipofuscinosis 1. In a specific embodiment, the disease or disorder is CLN2 disease or Jansky-Bielschowsky disease. In a specific embodiment, the disease or disorder is CLN3 disease. In a specific embodiment, the disease or disorder is CLN4 disease. In a specific embodiment, the disease or disorder is CLN5 disease. In a specific embodiment, the disease or disorder is CLN6 disease. In a specific embodiment, the disease or disorder is CLN7 disease. In a specific embodiment, the disease or disorder is CLN8 disease. In a specific embodiment, the disease or disorder is CLN10 disease. In a specific embodiment, the disease or disorder is CLN11 disease. In a specific embodiment, the disease or disorder is CLN12 disease. In a specific embodiment, the disease or disorder is CLN13 disease. In a specific embodiment, the disease or disorder is CLN14 disease. In a specific embodiment, the disease or disorder is cystinosis. In a specific embodiment, the disease or disorder is Danon disease. In a specific embodiment, the disease or disorder is Farber lipogranulomatosis. In a specific embodiment, the disease or disorder is fucosidosis. In a specific embodiment, the disease or disorder is galactosialidosis. In a specific embodiment, the disease or disorder is Gaucher disease. In a specific embodiment, the disease or disorder is glycogen storage disease type 2 (GSD II) or Pompe disease. In a specific embodiment, the disease or disorder is Griscelli syndrome 1 or Elejalde syndrome. In a specific embodiment, the disease or disorder is Griscelli syndrome 2. In a specific embodiment, the disease or disorder is Chéliak-Higashi disease. In a specific embodiment, the disease or disorder is GM1 gangliosidosis. In a specific embodiment, the disease or disorder is GM2 gangliosidosis Tay-Sachs disease. In a specific embodiment, the disease or disorder is GM2 gangliosidosis Sandhoff disease. In a specific embodiment, the disease or disorder is GM2 gangliosidosis GM2 activator deficiency. In a specific embodiment, the disease or disorder is Hermansky-Pudlak disease type 1. In a specific embodiment, the disease or disorder is Hermansky-Pudlak disease type 2. In a specific embodiment, the disease or disorder is Hermansky-Pudlak disease type 3. n a specific embodiment, the disease or disorder is Hermansky-Pudlak disease type 4. In a specific embodiment, the disease or disorder is Hermansky-Pudlak disease type 5. In a specific embodiment, the disease or disorder is Hermansky-Pudlak disease type 6. In a specific embodiment, the disease or disorder is Hermansky-Pudlak disease type 7. In a specific embodiment, the disease or disorder is Hermansky-Pudlak disease type 8. In a specific embodiment, the disease or disorder is Hermansky-Pudlak disease type 9. In a specific embodiment, the disease or disorder is Krabbe disease. In a specific embodiment, the disease or disorder is α-mannosidosisb. In a specific embodiment, the disease or disorder is β-mannosidosisb. In a specific embodiment, the disease or disorder is metachromatic leukodystrophy. In a specific embodiment, the disease or disorder is mucolipidosis II α/β or I-cell disease. In a specific embodiment, the disease or disorder is mucolipodosis III α/β or pseudo-Hurler polydystrophy. In a specific embodiment, the disease or disorder is mucolipidosis III y, variant pseudo-Hurler polydystrophy. In a specific embodiment, the disease or disorder is mucolipidosis IV. In a specific embodiment, the disease or disorder is mucopolysaccharidosis type I (MPS I) or Hurler syndrome. In a specific embodiment, the disease or disorder is mucopolysaccharidosis type II (MPS II) or Hunter syndrome. In a specific embodiment, the disease or disorder is mucopolysaccharidosis type IIIA (MPS IIIA) or Sanfilippo syndrome A. In a specific embodiment, the disease or disorder is mucopolysaccharidosis type IIB (MPS IIIB) or Sanfilippo syndrome B. In a specific embodiment, the disease or disorder is mucopolysaccharidosis type IIIC (MPS IIIC) or Sanfilippo syndrome C. In a specific embodiment, the disease or disorder is mucopolysaccharidosis type IIID (MPS IIID) or Sanfilippo syndrome D. In a specific embodiment, the disease or disorder is mucopolysaccharidosis type IVA (MPS IVA) or Morquio syndrome A. In a specific embodiment, the disease or disorder is mucopolysaccharidosis type IVB (MPS IVB) or Morquio syndrome B. In a specific embodiment, the disease or disorder is mucopolysaccharidosis type VI (MPS VI) or Maroteaux-Lamy syndrome. In a specific embodiment, the disease or disorder is mucopolysaccharidosis type VII (MPS VII) or Sly disease. In a specific embodiment, the disease or disorder is mucopolysaccharidosis type IX (MPS IX). In a specific embodiment, the disease or disorder is multiple sulfatase deficiency. In a specific embodiment, the disease or disorder is Niemann-Pick disease types A and B. In a specific embodiment, the disease or disorder is Niemann-Pick disease types C1. In a specific embodiment, the disease or disorder is Niemann-Pick disease types C2. In a specific embodiment, the disease or disorder is Salla disease. In a specific embodiment, the disease or disorder is Schindler disease or Kanzaki disease. In a specific embodiment, the disease or disorder is sialidosis type I or cherry-red spot myoclonus syndrome. In a specific embodiment, the disease or disorder is Wolman disease and cholesterol ester storage disease. In a specific embodiment, the disease or disorder is spinal muscular atrophy. In a specific embodiment, the disease or disorder is Duchenne muscular dystrophy (DMD). In a specific embodiment, the disease or disorder is phenylalanine hydroxylase deficiency. In a specific embodiment, the disease or disorder is hemophilia A. In a specific embodiment, the disease or disorder is hemophilia B.

In a specific embodiment, the disease or disorder is Fabry disease, and the biologic molecule is a protein that is α-galactosidase A (GLA) (preferably human GLA). In a specific embodiment, the disease or disorder is action myoclonus-renal failure syndrome, and the biologic molecule is a protein that is Scavenger Receptor Class B Member 2 (SCARB2) (preferably human SCARB2). In a specific embodiment, the disease or disorder is Aspartylglucos-aminuria, and the biologic molecule is a protein that is aspartylglucosaminidase (AGA) (preferably human AGA). In a specific embodiment, the disease or disorder is CLN1 disease or neuronal ceroid lipofuscinosis 1, and the biologic molecule is aprotein that is palmitoyl-protein thioesterase 1 (PPTI) (preferably human PPT1). In a specific embodiment, the disease or disorder is CLN2 disease or Jansky-Bielschowsky disease, and the biologic molecule is a protein that is tripeptidyl peptidase 1 (TPP1) (preferably human TPP1). In a specific embodiment, the disease or disorder is CLN3 disease, and the biologic molecule is a protein that is CLN3 lysosomal/endosomal transmembrane protein, battenin (CLN3) (preferably human CLN3). In a specific embodiment, the disease or disorder is CLN4 disease, and the biologic molecule is a protein that is DnaJ heat shock protein family (Hsp40) member C5 (DNAJC5) (preferably human DNAJC5). In a specific embodiment, the disease or disorder is CLN5 disease, and the biologic molecule is a protein that is CLN5 intracellular trafficking protein (CLN5) (preferably human CLN5). In a specific embodiment, the disease or disorder is CLN6 disease, and the biologic molecule is a protein that is CLN6 transmembrane ER protein (CLN6) (preferably human CLN6). In a specific embodiment, the disease or disorder is CLN7 disease, and the biologic molecule is a protein that is Major Facilitator Superfamily Domain Containing 8 (MFSD8) (preferably human MFSD8). In a specific embodiment, the disease or disorder is CLN8 disease, and the biologic molecule is a protein that is CLN8 transmembrane ER and ERGIC protein (CLN8) (preferably human CLN8). In a specific embodiment, the disease or disorder is CLN10 disease, and the biologic molecule is a protein that is cathepsin D (CTSD) (preferably human CTSD). In a specific embodiment, the disease or disorder is CLN11 disease, and the biologic molecule is a protein that is granulin precursor (GRN) (preferably human GRN). In a specific embodiment, the disease or disorder is CLN12 disease, and the biologic molecule is a protein that is ATPase cation transporting 13A2 (ATP13A2) (preferably human ATP13A2). In a specific embodiment, the disease or disorder is CLN13 disease, and the biologic molecule is a protein that is cathepsin F (CTSF) (preferably human CTSF). In a specific embodiment, the disease or disorder is CLN14 disease, and the biologic molecule is a protein that is Potassium Channel Tetramerization Domain Containing 7 (KCTD7) (preferably human KCTD7). In a specific embodiment, the disease or disorder is cystinosis, and the biologic molecule is a protein that is cystinosin, lysosomal cystine transporter (CTNS) (preferably human CTNS). In a specific embodiment, the disease or disorder is Danon disease, and the biologic molecule is a protein that is lysosomal associated membrane protein 2 (LAMP2) (preferably human LAMP2). In a specific embodiment, the disease or disorder is Farber lipogranulomatosis, and the biologic molecule is a protein that is N-acylsphingosine amidohydrolase 1 (ASAH1) (preferably human ASAH1). In a specific embodiment, the disease or disorder is fucosidosis, and the biologic molecule is a protein that is alpha-L-fucosidase 1 (FUCA1) (preferably human FUCA1). In a specific embodiment, the disease or disorder is galactosialidosis, and the biologic molecule is a protein that is cathepsin A (CTSA) (preferably human CTSA). In a specific embodiment, the disease or disorder is Gaucher disease, and the biologic molecule is a protein that is beta-glucocerebrosidase (GBA) (preferably human GBA). In a specific embodiment, the disease or disorder is glycogen storage disease type 2 (GSD II) or Pompe disease, and the biologic molecule is a protein that is acid alpha-glucosidase (GAA) (preferably human GAA). In a specific embodiment, the disease or disorder is Griscelli syndrome 1 or Elejalde syndrome, and the biologic molecule is a protein that is myosin VA (MYO5A) (preferably human MYO5A). In a specific embodiment, the disease or disorder is Griscelli syndrome 2, and the biologic molecule is a protein that is RAB27A, Member RAS Oncogene Family (RAB27A) (preferably human RAB27A). In a specific embodiment, the disease or disorder is Chdliak-Higashi disease, and the biologic molecule is aprotein that is Lysosomal Trafficking Regulator (LYST) (preferably human LYST). In a specific embodiment, the disease or disorder is GM1 gangliosidosis, and the biologic molecule is a protein that is galactosidase beta 1 (GLB1) (preferably human GLB1). In a specific embodiment, the disease or disorder is GM2 gangliosidosis Tay-Sachs disease, and the biologic molecule is a protein that is hexosaminidase subunit alpha (HEXA) (preferably human HEXA). In a specific embodiment, the disease or disorder is GM2 gangliosidosis Sandhoff disease, and the biologic molecule is a protein that is hexosaminidase subunit beta (HEXB) (preferably human HEXB). In a specific embodiment, the disease or disorder is GM2 gangliosidosis GM2 activator deficiency, and the biologic molecule is a protein that is ganglioside GM2 activator (GM2A) (preferably human GM2A). In a specific embodiment, the disease or disorder is Hermansky-Pudlak disease type 1, and the biologic molecule is a protein that is HPS1 biogenesis of lysosomal organelles complex 3 subunit 1 (HPS1) (preferably human HPS1). In a specific embodiment, the disease or disorder is Hermansky-Pudlak disease type 2, and the biologic molecule is a protein that is adaptor related protein complex 3 subunit beta 1 (AP3Bl or HPS2) (preferably human HPS2). In a specific embodiment, the disease or disorder is Hermansky-Pudlak disease type 3, and the biologic molecule is a protein that is HPS3 biogenesis of lysosomal organelles complex 2 subunit 1 (HPS3) (preferably human HPS3). In a specific embodiment, the disease or disorder is Hermansky-Pudlak disease type 4, and the biologic molecule is a protein that is HPS4 biogenesis of lysosomal organelles complex 3 subunit 2 (HPS4) (preferably human HPS4). In a specific embodiment, the disease or disorder is Hermansky-Pudlak disease type 5, and the biologic molecule is a protein that is HPS5 biogenesis of lysosomal organelles complex 2 subunit 2 (HPS5) (preferably human HPS5). In a specific embodiment, the disease or disorder is Hermansky-Pudlak disease type 6, and the biologic molecule is a protein that is HPS6 biogenesis of lysosomal organelles complex 2 subunit 3 (HPS6) (preferably human HPS6). In a specific embodiment, the disease or disorder is Hermansky-Pudlak disease type 7, and the biologic molecule is a protein that is dystrobrevin binding protein 1 (DTNBP1 or HPS7) (preferably human HPS7). In a specific embodiment, the disease or disorder is Hermansky-Pudlak disease type 8, and the biologic molecule is a protein that is biogenesis of lysosomal organelles complex 1 subunit 3 (BLOC1S3 or HPS8) (preferably human HPS8). In a specific embodiment, the disease or disorder is Hermansky-Pudlak disease type 9, and the biologic molecule is a protein that is biogenesis of lysosomal organelles complex 1 subunit 6 (BLOC1S6 or HPS9) (preferably human HPS9). In a specific embodiment, the disease or disorder is Krabbe disease, and the biologic molecule is a protein that is galactosylceramidase (GALC) (preferably human GALC). In a specific embodiment, the disease or disorder is α-mannosidosisb, and the biologic molecule is a protein that is mannosidase alpha class 2B member 1 (MAN2B1) (preferably human MAN2B1). In a specific embodiment, the disease or disorder is β-mannosidosisb, and the biologic molecule is a protein that is mannosidase beta (MANBA) (preferably human MANBA). In a specific embodiment, the disease or disorder is metachromatic leukodystrophy, and the biologic molecule is a protein that is arylsulfatase A (ARSA) (preferably human ARSA). In a specific embodiment, the disease or disorder is mucolipidosis II α/β or I-cell disease, and the biologic molecule is a protein that is N-acetylglucosamine-1-phosphate transferase subunits alpha and beta (GNPTAB) (preferably human GNPTAB). In a specific embodiment, the disease or disorder is mucolipodosis III a/B or pseudo-Hurler polydystrophy, and the biologic molecule is a protein that is N-acetylglucosamine-1-phosphate transferase subunits alpha and beta (GNPTAB) (preferably human GNPTAB). In a specific embodiment, the disease or disorder is mucolipidosis III y, variant pseudo-Hurler polydystrophy, and the biologic molecule is a protein that is N-acetylglucosamine-1-phosphate transferase subunit gamma (GNPTG) (preferably human GNPTG). In a specific embodiment, the disease or disorder is mucolipidosis IV, and the biologic molecule is a protein that is mucolipin TRP cation channel 1 (MCOLN1) (preferably human MCOLN1). In a specific embodiment, the disease or disorder is mucopolysaccharidosis type I (MPS I) or Hurler syndrome, and the biologic molecule is a protein that is alpha-L-iduronidase (IDUA) (preferably human IDUA). In a specific embodiment, the disease or disorder is mucopolysaccharidosis type II (MPS II) or Hunter syndrome, and the biologic molecule is a protein that is iduronate 2-sulfatase (IDS) (preferably human IDS). In a specific embodiment, the disease or disorder is mucopolysaccharidosis type IIIA (MPS IIIA) or Sanfilippo syndrome A, and the biologic molecule is a protein that is N-sulfoglucosamine sulfohydrolase (SGSH) (preferably human SGSH). In a specific embodiment, the disease or disorder is mucopolysaccharidosis type IIIB (MPS IIIB) or Sanfilippo syndrome B, and the biologic molecule is a protein that is N-acetyl-alpha-glucosaminidase (NAGLU) (preferably human NAGLU). In a specific embodiment, the disease or disorder is mucopolysaccharidosis type IIIC (MPS IIIC) or Sanfilippo syndrome C, and the biologic molecule is a protein that is heparan-alpha-glucosaminide N-acetyltransferase (HGSNAT) (preferably human HGSNAT). In a specific embodiment, the disease or disorder is mucopolysaccharidosis type IIID (MPS HID) or Sanfilippo syndrome D, and the biologic molecule is a protein that is glucosamine (N-acetyl)-6-sulfatase (GNS) (preferably human GNS). In a specific embodiment, the disease or disorder is mucopolysaccharidosis type IVA (MPS IVA) or Morquio syndrome A, and the biologic molecule is a protein that is galactosamine (N-acetyl)-6-sulfatase (GALNS) (preferably human GALNS). In a specific embodiment, the disease or disorder is mucopolysaccharidosis type IVB (MPS IVB) or Morquio syndrome B, and the biologic molecule is a protein that is galactosidase beta 1 (GLB1) (preferably human GLB1). In a specific embodiment, the disease or disorder is mucopolysaccharidosis type VI (MPS VI) or Maroteaux-Lamy syndrome, and the biologic molecule is a protein that is arylsulfatase B (ARSB) (preferably human ARSB). In a specific embodiment, the disease or disorder is mucopolysaccharidosis type VII (MPS VII) or Sly disease, and the biologic molecule is a protein that is beta-glucuronidase (GUSB) (preferably human GUSB). In a specific embodiment, the disease or disorder is mucopolysaccharidosis type IX (MPS IX), and the biologic molecule is a protein that is hyaluronidase 1 (HYAL1) (preferably human HYAL1). In a specific embodiment, the disease or disorder is multiple sulfatase deficiency, and the biologic molecule is a protein that is Sulfatase Modifying Factor 1 (SUMF1) (preferably human SUMF1). In a specific embodiment, the disease or disorder is Niemann-Pick disease types A and B, and the biologic molecule is a protein that is sphingomyelin phosphodiesterase 1 (SMPD1) (preferably human SMPD1). In a specific embodiment, the disease or disorder is Niemann-Pick disease types C1, and the biologic molecule is a protein that is NPC Intracellular Cholesterol Transporter 1 (NPC1) (preferably human NPC1). In a specific embodiment, the disease or disorder is Niemann-Pick disease types C2, and the biologic molecule is a protein that is NPC Intracellular Cholesterol Transporter 2 (NPC2) (preferably human NPC2). In a specific embodiment, the disease or disorder is Salla disease, and the biologic molecule is a protein that is Solute Carrier Family 17 Member 5 (SLC17A5) (preferably human SLC17A5). In a specific embodiment, the disease or disorder is Schindler disease or Kanzaki disease, and the biologic molecule is a protein that is alpha-N-acetylgalactosaminidase (NAGA) (preferably human NAGA). In a specific embodiment, the disease or disorder is sialidosis type I or cherry-red spot myoclonus syndrome, and the biologic molecule is a protein that is neuraminidase 1 (NEU1) (preferably human NEU1). In a specific embodiment, the disease or disorder is Wolman disease and cholesterol ester storage disease, and the biologic molecule is a protein that is lipase A, lysosomal acid type (LIPA) (preferably human LIPA). In a specific embodiment, the disease or disorder is spinal muscular atrophy, and the biologic molecule is a protein that is a Survival Motor Neuron (SMN) protein (preferably human SMN protein). In a specific embodiment, the disease or disorder is Duchenne muscular dystrophy (DMD), and the biologic molecule is a protein that is microdystrophin (preferably human microdystrophin). In a specific embodiment, the disease or disorder is phenylalanine hydroxylase deficiency, and the biologic molecule is a protein that is phenylalanine hydroxylase (PAH) (preferably human PAH). In a specific embodiment, the disease or disorder is hemophilia A, and the biologic molecule is a protein that is Coagulation Factor VIII (FVIII) (preferably human FVIII). In a specific embodiment, the disease or disorder is hemophilia B, and the biologic molecule is a protein that is Coagulation Factor IX (FIX) (preferably human FIX). In preferred embodiments, the biologic molecule is a functional protein (e.g., a wild-type protein).

In certain embodiments, the method is for the treatment of hereditary hemochromatosis (HH), a major disorder of iron overload, Wilson's disease, a genetic disorder of copper overload, or alpha1-antitrypsin deficiency. In certain embodiments, the biologic molecule is human Alpha1-antitrypsin (Accession: P01009. 3), HFE protein (Accession NP_000401. 1 or Q30201), or hepatic protein ATP7B (Accession P35670. 4) or variants with greater than 50, 60, 70, 80, 90, 95, or 95 sequence identity or similarity.

In certain embodiments, the method is for the treatment of hypercholesterolaemia. In certain embodiments, the biologic molecule is human phenylalanine hydroxylase (Accession: P00439. 1) or variants with greater than 50, 60, 70, 80, 90, 95, or 95 sequence identity or similarity.

In certain embodiments, the method is for the treatment of Type 1 tyrosinemia. In certain embodiments, the biologic molecule is human fumarylacetoacetate hydrolase (Accession: P16930. 2) or variants with greater than 50, 60, 70, 80, 90, 95, or 95 sequence identity or similarity.

In certain embodiments, the method is for the treatment of Type 2 tyrosinemia. In certain embodiments, the biologic molecule is human tyrosine aminotransferase (Accession: P17735. 1) or variants with greater than 50, 60, 70, 80, 90, 95, or 95 sequence identity or similarity.

In certain embodiments, the method is for the treatment of homocystinuria and hyperhomocysteinemia. In certain embodiments, the biologic molecule is human methylenetetrahydrofolate reductase (Accession: P42898. 3) or variants with greater than 50, 60, 70, 80, 90, 95, or 95 sequence identity or similarity.

In certain embodiments, the method is for the treatment of hyperlipidemia and hypercholesterolemia. In certain embodiments, the biologic molecule is human medium chain acyl-CoA dehydrogenase (Accession: P11310. 1) or variants with greater than 50, 60, 70, 80, 90, 95, or 95 sequence identity or similarity.

In certain embodiments, the method is for the treatment of Galactosemia. In certain embodiments, the biologic molecule is human galactose-1-phosphate uridyl transferase (Accession: P07902. 3) or variants with greater than 50, 60, 70, 80, 90, 95, or 95 sequence identity or similarity.

In certain embodiments, the method is for the treatment of Lesch-Nyhan syndrome. In certain embodiments, the biologic molecule is human hypoxanthine phosphoribosyl-transferase (Accession: P00492. 2) or variants with greater than 50, 60, 70, 80, 90, 95, or 95 sequence identity or similarity.

In certain embodiments, the method is for the treatment of Gaucher disease. In certain embodiments, the biologic molecule is human cerebrosidase (Accession: P07602. 2, Accession: P04062. 3) or variants with greater than 50, 60, 70, 80, 90, 95, or 95 sequence identity or similarity.

In certain embodiments, the method is for the treatment of Tay-Sachs disease. In certain embodiments, the biologic molecule is human beta-hexosaminidase A (Accession: P06865. 2) or variants with greater than 50, 60, 70, 80, 90, 95, or 95 sequence identity or similarity.

In certain embodiments, the method is for the treatment of Fabry disease. In certain embodiments, the biologic molecule is human alpha-galactosidase (Accession: P06280. 1) or variants with greater than 50, 60, 70, 80, 90, 95, or 95 sequence identity or similarity.

In certain embodiments, the method is for the treatment of Hunter syndrome. In certain embodiments, the biologic molecule is human iduronate sulphatase (Accession: P22304. 1) or variants with greater than 50, 60, 70, 80, 90, 95, or 95 sequence identity or similarity.

In certain embodiments, the method is for the treatment of glycogen storage disease type Ta. In certain embodiments, the biologic molecule is human glucose-6-phosphatase (Accession: P35575. 2) or variants with greater than 50, 60, 70, 80, 90, 95, or 95 sequence identity or similarity.

In certain embodiments, the method is for the treatment of ammonia metabolism. In certain embodiments, the biologic molecule is human ornithine transcarbamylase (Accession: P00480. 3) or variants with greater than 50, 60, 70, 80, 90, 95, or 95 sequence identity or similarity.

In certain embodiments, the method is for the treatment of phenylketonuria. In certain embodiments, the biologic molecule is human low-density lipoprotein receptor (Accession: P01130. 1) or variants with greater than 50, 60, 70, 80, 90, 95, or 95 sequence identity or similarity.

In certain embodiments, the method is for the treatment of propionic acidemia. In certain embodiments, the biologic molecule is human propionyl-coenzyme A carboxylase, either PCCA and/or PCCB (Accession: P05166. 3 beta, NP_000273. 2 alpha, NP_001121164. 1 alpha) or variants with greater than 50, 60, 70, 80, 90, 95, or 95 sequence identity or similarity.

### 5.8 Assays

5.8.1 Analysis of mRNA Levels

The change of levels or expression of a gene can be assayed in a variety of ways known in the art.

For example, several methods of detecting or quantitating mRNA levels are known in the art. Exemplary methods include, but are not limited to, northern blots, ribonuclease protection assays, PCR-based methods, and the like. The mRNA sequence of a gene can be used to prepare a probe that is at least partially complementary to the mRNA sequence. The probe can then be used to detect the mRNA in a sample, using any suitable assay, such as PCR based methods, northern blotting, a dipstick assay, and the like.

The assay method can be varied depending on the type of mRNA information desired. Exemplary methods include but are not limited to northern blots and PCR-based methods (e.g., qRT-PCR). Methods such as qRT-PCR can also accurately quantitate the amount of the mRNA in a sample.

Any suitable assay platform can be used to determine the presence of mRNA in a sample. For example, an assay may be in the form of a dipstick, a membrane, a chip, a disk, a test strip, a filter, a microsphere, a slide, a multi-well plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film, a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.). See, e.g., Ausubel et al., Short Protocols in Molecular Biology (Wiley & Sons, 3rd ed. 1995); Sambrook et al., Molecular Cloning: A Laboratory Manual (Cold Spring Harbor, N.Y., 3rd ed. 2001). In some embodiments, the sample is labeled with fluorescent label. Exemplary fluorescent dyes include, but are not limited to, xanthene dyes, fluorescein dyes (e.g., fluorescein isothiocyanate (FITC), 6-carboxyfluorescein (FAM), 6 carboxy-2′,4′,7′,4,7-hexachlorofluorescein (HEX), 6-carboxy-4′,5′-dichloro-2′,7′-dimethoxyfluorescein (JOE)), rhodamine dyes (e.g., rhodamine 110 (R110), N,N,N′,N′-tetramethyl-6-carboxyrhodamine (TAMRA), 6-carboxy-X-rhodamine (ROX), 5 carboxyrhodamine 6G (R6G5 or G5), 6-carboxyrhodamine 6G (R6G6 or G6)), cyanine dyes (e.g., Cy3, Cy5 and Cy7), Alexa dyes (e.g., Alexa-fluor-555), coumarin, Diethylaminocoumarin, umbelliferone, benzimide dyes (e.g., Hoechst 33258), phenanthridine dyes (e.g., Texas Red), ethidium dyes, acridine dyes, carbazole dyes, phenoxazine dyes, porphyrin dyes, polymethine dyes, BODIPY dyes, quinoline dyes, Pyrene, Fluorescein Chlorotriazinyl, eosin dyes, Tetramethylrhodamine, Lissamine, Napthofluorescein, and the like.

A typical mRNA assay method can contain the steps of 1) obtaining surface-bound subject probes; 2) hybridizing a population of mRNAs to the surface-bound probes under conditions sufficient to provide for specific binding; (3) post-hybridization washing to remove nucleic acids not specifically bound to the surface-bound probes; and (4) detecting the hybridized mRNAs. The reagents used in each of these steps and their conditions for use may vary depending on the particular application.

Hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions may be employed.

Hybridization is typically performed under stringent hybridization conditions. Standard hybridization techniques (e.g., under conditions sufficient to provide for specific binding of target mRNAs in the sample to the probes) are described in Kallioniemi et al., Science, 258:818-821 (1992) and International Patent Application Publication No. WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al., Meth. Enzymol. 1981, 21:470-480; Angerer et al., Genetic Engineering: Principles and Methods, Vol 7, pgs 43-65 (Plenum Press, New York, Setlow and Hollaender, eds. 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art.

Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

Other methods, such as PCR-based methods, can also be used to detect the expression of a gene. Examples of PCR methods can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, quantitative Reverse Transcription-PCR (qRT-PCR) can be used for both the detection and quantification of RNA targets (Bustin et al., Clin. Sci. 2005, 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663, which is incorporated by reference herein in its entirety.

In contrast to regular reverse transcriptase-PCR and analysis by agarose gels, qRT PCR gives quantitative results. An additional advantage of qRT-PCR is the relative ease and convenience of use. Instruments for qRT-PCR, such as the Applied Biosystems 7500, are available commercially, so are the reagents, such as TaqMan® Sequence Detection Chemistry. For example, TaqMan® Gene Expression Assays can be used, following the manufacturer's instructions. These kits are pre-formulated gene expression assays for rapid, reliable detection and quantification of human, mouse, and rat mRNA transcripts. To determine the cycle number at which the fluorescence signal associated with a particular amplicon accumulation crosses the threshold (referred to as the CT), the data can be analyzed, for example, using 7500 Real-Time PCR System Sequence Detection software vs. using the comparative CT relative quantification calculation method. Using this method, the output is expressed as a fold-change of expression levels. In some embodiments, the threshold level can be selected to be automatically determined by the software. In some embodiments, the threshold level is set to be above the baseline but sufficiently low to be within the exponential growth region of an amplification curve.

In other embodiments, a target RNA can be detected or quantified by Next Generation Sequencing (NGS).

### 5.8.2 Analysis of Protein Levels

The change of protein expression level can be assessed by measuring levels of protein of interest. Protein levels can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS), LC-MS (Liquid-chromatography-MassSpec), and other methods. Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies useful for the detection of mouse, rat, monkey, and human protein of interest are commercially available. In case of MassSpec, protein levels can be measure using labeled or unlabeled methods.

### 5.8.3 In Vivo Analysis

In vivo assays can be used to assess the treatment efficacy of a recombinant viral particle, including a recombinant AAV particle, a non-viral gene delivery system, or a pharmaceutical composition of the present disclosure.

In some embodiments, motor function is measured by righting, open field performance in the animal. In certain embodiments, respiration is measured by whole body plethysmograph, invasive resistance, and compliance measurements in the animal.

In some embodiments, overall survival (OS) and disease free survival (DFS) is measured by body weight, health status observation twice daily in the alive animal.

Testing may be performed in normal animals, or in experimental disease models. For administration to animals, oligonucleotides can be formulated in a pharmaceutically acceptable diluent, such as phosphate-buffered saline. Administration includes parenteral routes of administration, such as intraperitoneal, intravenous, and subcutaneous. Calculation of the dosage and dosing frequency of a recombinant viral particle, including a recombinant AAV particle, a non-viral gene delivery system, or a pharmaceutical composition of the present disclosure is within the abilities of those skilled in the art, and depends upon factors such as route of administration and animal body weight. Following a period of treatment with a recombinant viral particle, including a recombinant AAV particle, a non-viral gene delivery system, or a pharmaceutical composition of the present disclosure, RNA or protein can be isolated from tissues of interest, including liver, muscle, kidney, spleen, CNS tissue or CSF and changes in the expression of the protein of interest are measured, e.g., using NGS.

### 5.9 Kits and Articles of Manufacture

Further provided are kits, unit dosages, and articles of manufacture comprising any of the compositions described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### Sequences

TABLE 1
Table of Sequences.

| SEQ | | |
|---|---|---|
| ID | | |
| NO. | Description | SEQUENCE |
| 1 | Codon-optimized human α- | ATGCAGCTGAGGAACCCTGAGCTGCACCTGGGCTGTG |
| | galactosidase A nucleotide | CCCTGGCCCTGAGGTTCCTGGCCCTGGTGAGCTGGGA |
| | sequence #1 (hGLAco1) | CATCCCCGGGGCTAGAGCCCTGGACAATGGCCTGGCT |
| | | AGGACCCCCACCATGGGCTGGCTGCACTGGGAGAGG |
| | | TTCATGTGCAACCTGGACTGCCAAGAGGAGCCTGACA |
| | | GCTGCATCAGTGAGAAGCTGTTCATGGAGATGGCTGA |
| | | GCTGATGGTGTCAGAGGGCTGGAAGGATGCCGGCTAT |
| | | GAGTACCTGTGCATTGATGACTGCTGGATGGCCCCTC |
| | | AGAGAGACTCAGAAGGCAGACTGCAAGCTGACCCTC |
| | | AGAGGTTCCCCCATGGCATCAGGCAGCTGGCCAACTA |
| | | TGTGCACAGCAAGGGCCTGAAGCTGGGCATCTATGCA |
| | | GATGTGGGCAACAAGACCTGTGCCGGCTTCCCCGGCA |
| | | GCTTTGGCTACTATGACATTGATGCCCAGACCTTTGCT |
| | | GACTGGGGAGTGGACCTGCTGAAGTTTGATGGCTGCT |
| | | ACTGTGACAGCCTGGAGAACCTGGCAGATGGCTACA |
| | | AGCACATGAGCCTGGCCCTGAACAGGACCGGCAGAA |
| | | GCATTGTGTACAGCTGTGAGTGGCCCCTGTACATGTG |
| | | GCCCTTTCAGAAGCCCAACTACACAGAGATCAGACAG |
| | | TACTGCAACCACTGGAGGAACTTTGCTGACATTGATG |
| | | ACAGCTGGAAGAGCATCAAGAGCATCCTGGACTGGA |
| | | CAAGCTTCAACCAAGAGAGGATTGTGGATGTGGCCG |
| | | GCCCCGGCGGCTGGAATGACCCTGACATGCTGGTGAT |
| | | TGGCAACTTTGGCCTGAGCTGGAATCAGCAAGTGACA |
| | | CAGATGGCCCTGTGGGCCATCATGGCTGCCCCCCTGT |
| | | TCATGAGCAATGACCTGAGGCACATCAGCCCCCAAGC |
| | | CAAGGCCCTGCTGCAAGACAAGGATGTGATTGCCATC |
| | | AACCAAGACCCCCTGGGCAAGCAAGGCTATCAGCTG |
| | | AGGCAAGGAGACAACTTTGAAGTGTGGGAGAGACCC |
| | | CTGAGCGGCCTGGCCTGGGCTGTGGCCATGATCAACA |
| | | GGCAAGAGATTGGCGGCCCTAGAAGCTACACCATTGC |
| | | TGTGGCTAGCCTGGGCAAGGGAGTGGCCTGCAACCCT |
| | | GCCTGCTTCATCACACAGCTGCTGCCTGTGAAGAGGA |
| | | AGCTGGGCTTCTATGAGTGGACAAGCAGGCTGAGATC |
| | | ACACATCAACCCCACCGGCACTGTGCTGCTGCAGCTG |
| | | GAGAACACCATGCAGATGAGCCTGAAGGACCTGCTG |
| | | TGA |
| 2 | Codon-optimized human α- | ATGCAGCTGAGGAACCCAGAGCTGCACCTGGGCTGTG |
| | galactosidase A nucleotide | CCCTGGCCCTCAGGTTTCTGGCCCTGGTGTCCTGGGA |
| | sequence #2 (hGLAco2) | CATCCCTGGGGCCAGAGCCCTGGACAATGGCCTGGCT |
| | | AGAACCCCTACCATGGGCTGGCTGCACTGGGAGCGGT |
| | | TCATGTGCAACCTGGACTGCCAGGAGGAACCAGACTC |
| | | CTGCATCTCTGAGAAGCTGTTCATGGAGATGGCAGAG |
| | | CTGATGGTCTCTGAGGGATGGAAGGATGCCGGCTATG |
| | | AGTACCTGTGCATTGATGACTGCTGGATGGCCCCTCA |
| | | GCGGGACTCTGAAGGCAGACTGCAGGCAGACCCTCA |
| | | GCGGTTCCCTCATGGCATCCGGCAGCTGGCTAACTAT |
| | | GTGCACAGCAAGGGCCTGAAGCTGGGAATCTATGCA |
| | | GATGTGGGCAACAAGACCTGTGCCGGATTCCCTGGCT |
| | | CTTTTGGATATTATGACATTGATGCCCAGACCTTTGCT |
| | | GATTGGGGAGTGGACCTGCTGAAGTTTGATGGCTGCT |
| | | ACTGTGATAGCCTGGAAAACCTGGCTGATGGCTACAA |
| | | GCACATGAGCCTGGCCCTGAACCGGACAGGCAGAAG |
| | | CATTGTGTACAGCTGTGAGTGGCCACTGTACATGTGG |
| | | CCATTCCAGAAGCCCAACTACACAGAGATCAGACAGT |
| | | ACTGCAACCACTGGCGGAATTTTGCAGATATTGATGA |
| | | CAGCTGGAAGTCCATCAAGAGCATCCTGGACTGGACC |
| | | AGCTTCAACCAAGAAAGAATTGTGGATGTGGCCGGCC |
| | | CTGGCGGCTGGAATGATCCAGATATGCTGGTCATTGG |
| | | CAACTTTGGCCTGAGCTGGAACCAGCAGGTGACCCAG |
| | | ATGGCCCTGTGGGCCATCATGGCTGCCCCTCTGTTTAT |
| | | GAGCAATGACCTGAGACACATCAGCCCTCAGGCCAA |
| | | GGCCCTGCTGCAGGACAAGGATGTGATTGCAATCAAC |
| | | CAGGACCCTCTGGGCAAACAGGGCTATCAGCTGAGA |
| | | CAAGGGGACAACTTTGAGGTGTGGGAAAGACCTCTG |
| | | AGCGGCCTGGCTTGGGCTGTGGCCATGATCAATAGAC |
| | | AGGAGATTGGCGGACCTAGATCCTACACCATTGCTGT |
| | | GGCCAGCCTGGGCAAGGGAGTGGCTTGTAATCCTGCC |
| | | TGTTTTATCACCCAGCTGCTCCCTGTTAAGAGAAAGC |
| | | TGGGCTTCTATGAGTGGACCAGCCGGCTGAGATCTCA |
| | | CATCAACCCTACAGGCACAGTGCTGCTGCAGCTGGAA |
| | | AACACCATGCAGATGAGCCTGAAAGACCTGCTGTGA |
| 3 | Short hepatocyte specific | GGCAAAGTCCATATTGACTTAGGTTAATCATTAACTC |
| | promoter (sH) nucleotide | TTTGTTGACTAAGTCAATAATC |
| | sequence | |
| 4 | Hepatocyte-cardiomyocyte bi- | GGCAAAGTCCATATTGACTTAGGTTAATCATTAACTC |
| | specific promoter HC1 nucleotide | TTTGTTGACTAAGTCAATAATCggggttatttttaaa |
| | sequence | cTATCTGCTCATCTACTTCCGGTctttccattcAGGA |
| | | ATGcctttcttgggggggcggggCTATAAAAGCCCCA |
| | | GGCTGGGAGCAGCCATCACAGAAGTCCACTCATTCTT |
| 5 | Hepatocyte-cardiomyocyte bi- | GGCAAAGTCCATATTGACTTAGGTTAATCATTAACTC |
| | specific promoter HC2 nucleotide | TTTGTTGACTAAGTCAATAATCCTGTGGAATGTGAGG |
| | sequence | CCTGGCCTGGGAGATATTTTTGCTGCACTTTGAGCCA |
| | | CCCCGCCCCCTGGAACTCAGACCCTGCACAGTCCATG |
| | | CCATAACGGGGGCGGGGCTATAAAAGCCCCAGGCTG |
| | | GGAGCAGCCATCACAGAAGTCCACTCATTCTT |
| 6 | Hepatocyte-cardiomyocyte bi- | GGTTAAAAATAACACTATCTGCTCATCAGGAGGAATG |
| | specific promoter HC3 nucleotide | TGCCTAAAAAAAGGGACCTTTCTTGGGCTCCTCCTGT |
| | sequence | CACCTCCAGAGCCAAAGGAGGAGGAGCCACTCCAAA |
| | | TTTAGAGGCAAAGTCCATATTGACTTAGGTTAATCAT |
| | | TAACTCTTTGTTGACTAAGTCAATAATCAGAATCAGC |
| | | AGGTTTGCAGTCAGATTGGCAGTATAAAAGCCCCAGG |
| | | CTGGGAGCAGCCATCACAGAAGTCCACTCATTCTT |
| 7 | Hepatocyte-cardiomyocyte bi- | GGTTAAAAATAACTGAGGTAAGGGCCATGGCAGGGT |
| | specific promoter HC4 nucleotide | GGGAGAGGCGGTGTGAGAAGGTCCTGTCTTCCCACTA |
| | sequence | TCTGCTCATCAGCCCTTTGAAGGGGAGGAATGTGCCC |
| | | AAGGACTAAAAAAAGGCCGTGGAGCCAGAGAGGCTG |
| | | GGGCAGCAGACCTTTCTTGGGCGGCAAAGTCCATATT |
| | | GACTTAGGTTAATCATTAACTCTTTGTTGACTAAGTCA |
| | | ATAATCAGAATCAGCAGGTTTGCAGTCAGATTGGCAG |
| | | TATAAAAGCCCCAGGCTGGGAGCAGCCATCACAGAA |
| | | GTCCACTCATTCTT |
| 8 | Hepatocyte-cardiomyocyte bi- | GGACCACATGGGCTTATATGGCGTGGGGTACATGTTC |
| | specific promoter HC5 nucleotide | CTGTAGCCTTGTCCCTGGCACCTGCCAAAATAGCAGC |
| | sequence | CAACACCCCCCACCCCCACCGCCATCCCCCTGCCCCA |
| | | CCCGTCCCCTGTCGCACATTCCTCCGGCAAAGTCCAT |
| | | ATTGACTTAGGTTAATCATTAACTCTTTGTTGACTAAG |
| | | TCAATAATCAGAATCAGCAGGTTTGCAGTCAGATTGG |
| | | CAGTATAAAAGCCCCAGGCTGGGAGCAGCCATCACA |
| | | GAAGTCCACTCATTCTT |
| 9 | Hepatocyte-myocyte bi-specific | GGCAAAGTCCATATTGACTTAGGTTAATCATTAACTC |
| | promoter HC6 (sH + truncated | TTTGTTGACTAAGTCAATAATCGCTGCCCTTGTAAGG |
| | and modified human MCK) | AGGCGAGATCTAGAGACACCCGAGACGCCCGGTTAT |
| | nucleotide sequence | AATTAACCAGGACACGTGGCGAACCCCCCTCCAACAC |
| | | CTGCCCCCTCTAAAAATAACTCCTTTCCCGGCGGGGG |
| | | CCAGCTGTCCCCACCGCCAGGGGGCGGGGCTATAAA |
| | | AGCCCCAGGCTGGGAGCAGCCATCACAGAAGTCCAC |
| | | TCATTCTT |
| 10 | Hepatocyte-myocyte bi-specific | GGCAAAGTCCATATTGACTTAGGTTAATCATTAACTC |
| | promoter HC7 (sH + MCK-D3) | TTTGTTGACTAAGTCAATAATCCTCGAGTGCCCATGT |
| | nucleotide sequence | AAGGAGGCAAGGCCTGGGGACACCCGAGATGCCTGG |
| | | TTATAATTAACCCAGACATGTGGCTGCCCCCCCCCCC |
| | | CCAACACCTGCTGCCTCTAAAAATAACCCTGCATGCC |
| | | ATGTTCCCGGCGAAGGGCCAGCTGTCCCCCGCGGGCA |
| | | GCCCATACAAGGCCATGGGGCTGGGCAAGCTGCACG |
| | | CCAGGGGCCCCTCCCTGGGGACAGCCCCTCCTGGCTA |
| | | GTCACACCCTGTAGGCTCCTCTATATAACCCAGGGGC |
| | | ACAGGGGCTGCCCTCATTCTACCACCACCTCCACAGC |
| | | ACAGACAGACACTCAGGAGCCAGCCAGC |
| 11 | Truncated mouse muscle creatine | TGCCCATGTAAGGAGGCAAGGCCTGGGGACACCCGA |
| | kinase (MCK) promoter MCK- | GATGCCTGGTTATAATTAACCCAGACATGTGGCTGCC |
| | D1 nucleotide sequence | CCCCCCCCCCCAACACCTGCTGCCTCTAAAAATAACC |
| | | CTGCATGCCATGTTCCCGGCGAAGGGCCAGCTGTCCC |
| | | CCGCGGGCAGCCCATACAAGGCCATGGGGCTGGGCA |
| | | AGCTGCACGCCTGGGTCCGGGGTGGGCACGGTGCCCG |
| | | GGCAACGAGCTGAAAGCTCATCTGCTCTCAGGGGCCC |
| | | CTCCCTGGGGACAGCCCCTCCTGGCTAGTCACACCCT |
| | | GTAGGCTCCTCTATATAACCCAGGGGCACAGGGGCTG |
| | | CCCTCATTCTACCACCACCTCCACAGCACAGACAGAC |
| | | ACTCAGGAGCCAGCCAGC |
| 12 | Truncated mouse muscle creatine | TGCCCATGTAAGGAGGCAAGGCCTGGGGACACCCGA |
| | kinase (MCK) promoter MCK- | GATGCCTGGTTATAATTAACCCAGACATGTGGCTGCC |
| | D2 nucleotide sequence | CCCCCCCCCCCAACACCTGCTGCCTCTAAAAATAACC |
| | | CTGCATGCCATGTTCCCGGCGAAGGGCCAGCTGTCCC |
| | | CCGCCAGCTAGACTCAGCACTTAGTTTAGGAACCAGT |
| | | GAGCAAGTCAGCCCTTGGGGCAGCCCATACAAGGCC |
| | | ATGGGGCTGGGCAAGCTGCACGCCAGGGGCCCCTCCC |
| | | TGGGGACAGCCCCTCCTGGCTAGTCACACCCTGTAGG |
| | | CTCCTCTATATAACCCAGGGGCACAGGGGCTGCCCTC |
| | | ATTCTACCACCACCTCCACAGCACAGACAGACACTCA |
| | | GGAGCCAGCCAGC |
| 13 | Truncated mouse muscle creatine | TGCCCATGTAAGGAGGCAAGGCCTGGGGACACCCGA |
| | kinase (MCK) promoter MCK- | GATGCCTGGTTATAATTAACCCAGACATGTGGCTGCC |
| | D3 nucleotide sequence | CCCCCCCCCCCAACACCTGCTGCCTCTAAAAATAACC |
| | | CTGCATGCCATGTTCCCGGCGAAGGGCCAGCTGTCCC |
| | | CCGCGGGCAGCCCATACAAGGCCATGGGGCTGGGCA |
| | | AGCTGCACGCCAGGGGCCCCTCCCTGGGGACAGCCCC |
| | | TCCTGGCTAGTCACACCCTGTAGGCTCCTCTATATAAC |
| | | CCAGGGGCACAGGGGCTGCCCTCATTCTACCACCACC |
| | | TCCACAGCACAGACAGACACTCAGGAGCCAGCCAGC |
| 14 | CK8e promoter nucleotide | TGCCCATGTAAGGAGGCAAGGCCTGGGGACACCCGA |
| | sequence | GATGCCTGGTTATAATTAACCCAGACATGTGGCTGCC |
| | | CCCCCCCCCCCAACACCTGCTGCCTCTAAAAATAACC |
| | | CTGCATGCCATGTTCCCGGCGAAGGGCCAGCTGTCCC |
| | | CCGCCAGCTAGACTCAGCACTTAGTTTAGGAACCAGT |
| | | GAGCAAGTCAGCCCTTGGGGCAGCCCATACAAGGCC |
| | | ATGGGGCTGGGCAAGCTGCACGCCTGGGTCCGGGGT |
| | | GGGCACGGTGCCCGGGCAACGAGCTGAAAGCTCATC |
| | | TGCTCTCAGGGGCCCCTCCCTGGGGACAGCCCCTCCT |
| | | GGCTAGTCACACCCTGTAGGCTCCTCTATATAACCCA |
| | | GGGGCACAGGGGCTGCCCTCATTCTACCACCACCTCC |
| | | ACAGCACAGACAGACACTCAGGAGCCAGCCAGC |
| 15 | Truncated and modified human | gctgcccttgtaaggaggcgagATcTAGAgacaccc |
| | muscle creatine kinase (MCK) | gagacgcccggttataattaaccaggacacgtggcg |
| | promoter MCK-D4 nucleotide | aacccccctccaacacctgccccctctaaaaataac |
| | sequence | tcctttcccggcgggggccagctgtccccaccgcca |
| 16 | Nucleotide sequence of human α- | GGCAAAGTCCATATTGACTTAGGTTAATCATTAACTCT |
| | galactosidase A expression | TTGTTGACTAAGTCAATAATCGGGGTTATTTTTAAACTA |
| | cassette containing promoter | TCTGCTCATCTACTTCCGGTCTTTCCATTCAGGAATGC |
| | HC1, hGLAco1 and bovine | CTTTCTTGGGGGGGCGGGGCTATAAAAGCCCCAGGCT |
| | growth hormone (bGH) poly A | GGGAGCAGCCATCACAGAAGTCCACTCATTCTTGCGAT |
| | sequences (Cassette 1) | CGCCTCGAGAGAATTCGCCACCATGCAGCTGAGGAAC |
| | | CCTGAGCTGCACCTGGGCTGTGCCCTGGCCCTGAGGT |
| | | TCCTGGCCCTGGTGAGCTGGGACATCCCCGGGGCTAG |
| | | AGCCCTGGACAATGGCCTGGCTAGGACCCCCACCATG |
| | | GGCTGGCTGCACTGGGAGAGGTTCATGTGCAACCTGG |
| | | ACTGCCAAGAGGAGCCTGACAGCTGCATCAGTGAGA |
| | | AGCTGTTCATGGAGATGGCTGAGCTGATGGTGTCAGA |
| | | GGGCTGGAAGGATGCCGGCTATGAGTACCTGTGCATT |
| | | GATGACTGCTGGATGGCCCCTCAGAGAGACTCAGAA |
| | | GGCAGACTGCAAGCTGACCCTCAGAGGTTCCCCCATG |
| | | GCATCAGGCAGCTGGCCAACTATGTGCACAGCAAGG |
| | | GCCTGAAGCTGGGCATCTATGCAGATGTGGGCAACAA |
| | | GACCTGTGCCGGCTTCCCCGGCAGCTTTGGCTACTAT |
| | | GACATTGATGCCCAGACCTTTGCTGACTGGGGAGTGG |
| | | ACCTGCTGAAGTTTGATGGCTGCTACTGTGACAGCCT |
| | | GGAGAACCTGGCAGATGGCTACAAGCACATGAGCCT |
| | | GGCCCTGAACAGGACCGGCAGAAGCATTGTGTACAG |
| | | CTGTGAGTGGCCCCTGTACATGTGGCCCTTTCAGAAG |
| | | CCCAACTACACAGAGATCAGACAGTACTGCAACCACT |
| | | GGAGGAACTTTGCTGACATTGATGACAGCTGGAAGA |
| | | GCATCAAGAGCATCCTGGACTGGACAAGCTTCAACCA |
| | | AGAGAGGATTGTGGATGTGGCCGGCCCCGGCGGCTG |
| | | GAATGACCCTGACATGCTGGTGATTGGCAACTTTGGC |
| | | CTGAGCTGGAATCAGCAAGTGACACAGATGGCCCTGT |
| | | GGGCCATCATGGCTGCCCCCCTGTTCATGAGCAATGA |
| | | CCTGAGGCACATCAGCCCCCAAGCCAAGGCCCTGCTG |
| | | CAAGACAAGGATGTGATTGCCATCAACCAAGACCCCC |
| | | TGGGCAAGCAAGGCTATCAGCTGAGGCAAGGAGACA |
| | | ACTTTGAAGTGTGGGAGAGACCCCTGAGCGGCCTGGC |
| | | CTGGGCTGTGGCCATGATCAACAGGCAAGAGATTGGC |
| | | GGCCCTAGAAGCTACACCATTGCTGTGGCTAGCCTGG |
| | | GCAAGGGAGTGGCCTGCAACCCTGCCTGCTTCATCAC |
| | | ACAGCTGCTGCCTGTGAAGAGGAAGCTGGGCTTCTAT |
| | | GAGTGGACAAGCAGGCTGAGATCACACATCAACCCC |
| | | ACCGGCACTGTGCTGCTGCAGCTGGAGAACACCATGC |
| | | AGATGAGCCTGAAGGACCTGCTGTGAGCGGCCGCCTG |
| | | TGCCTTCTAGTTGCCAGCCATCTGTTGTTTGCCCCTCCCC |
| | | CGTGCCTTCCTTGACCCTGGAAGGTGCCACTCCCACTGT |
| | | CCTTTCCTAATAAAATGAGGAAATTGCATCGCATTGTCTG |
| | | AGTAGGTGTCATTCTATTCTGGGGGGTGGGGTGGGGCA |
| | | GGACAGCAAGGGGGAGGATTGGGAAGACAATAGCAGGC |
| | | ATGCTGGGGA |
| 17 | Nucleotide sequence of human α- | GGCAAAGTCCATATTGACTTAGGTTAATCATTAACTCT |
| | galactosidase A expression | TTGTTGACTAAGTCAATAATCGCTGCCCTTGTAAGGAG |
| | cassette containing promoter | GCGAGATCTAGAGACACCCGAGACGCCCGGTTATAATT |
| | HC6, hGLAco1 and bovine | AACCAGGACACGTGGCGAACCCCCCTCCAACACCTGC |
| | growth hormone (bGH) poly A | CCCCTCTAAAAATAACTCCTTTCCCGGCGGGGGCCAGC |
| | sequences (Cassette 2) | TGTCCCCACCGCCAGGGGGCGGGGCTATAAAAGCCCC |
| | | AGGCTGGGAGCAGCCATCACAGAAGTCCACTCATTCTT |
| | | GCGATCGCCTCGAGAGAATTCGCCACCATGCAGCTGA |
| | | GGAACCCTGAGCTGCACCTGGGCTGTGCCCTGGCCCT |
| | | GAGGTTCCTGGCCCTGGTGAGCTGGGACATCCCCGGG |
| | | GCTAGAGCCCTGGACAATGGCCTGGCTAGGACCCCCA |
| | | CCATGGGCTGGCTGCACTGGGAGAGGTTCATGTGCAA |
| | | CCTGGACTGCCAAGAGGAGCCTGACAGCTGCATCAGT |
| | | GAGAAGCTGTTCATGGAGATGGCTGAGCTGATGGTGT |
| | | CAGAGGGCTGGAAGGATGCCGGCTATGAGTACCTGT |
| | | GCATTGATGACTGCTGGATGGCCCCTCAGAGAGACTC |
| | | AGAAGGCAGACTGCAAGCTGACCCTCAGAGGTTCCCC |
| | | CATGGCATCAGGCAGCTGGCCAACTATGTGCACAGCA |
| | | AGGGCCTGAAGCTGGGCATCTATGCAGATGTGGGCA |
| | | ACAAGACCTGTGCCGGCTTCCCCGGCAGCTTTGGCTA |
| | | CTATGACATTGATGCCCAGACCTTTGCTGACTGGGGA |
| | | GTGGACCTGCTGAAGTTTGATGGCTGCTACTGTGACA |
| | | GCCTGGAGAACCTGGCAGATGGCTACAAGCACATGA |
| | | GCCTGGCCCTGAACAGGACCGGCAGAAGCATTGTGTA |
| | | CAGCTGTGAGTGGCCCCTGTACATGTGGCCCTTTCAG |
| | | AAGCCCAACTACACAGAGATCAGACAGTACTGCAAC |
| | | CACTGGAGGAACTTTGCTGACATTGATGACAGCTGGA |
| | | AGAGCATCAAGAGCATCCTGGACTGGACAAGCTTCA |
| | | ACCAAGAGAGGATTGTGGATGTGGCCGGCCCCGGCG |
| | | GCTGGAATGACCCTGACATGCTGGTGATTGGCAACTT |
| | | TGGCCTGAGCTGGAATCAGCAAGTGACACAGATGGC |
| | | CCTGTGGGCCATCATGGCTGCCCCCCTGTTCATGAGC |
| | | AATGACCTGAGGCACATCAGCCCCCAAGCCAAGGCC |
| | | CTGCTGCAAGACAAGGATGTGATTGCCATCAACCAAG |
| | | ACCCCCTGGGCAAGCAAGGCTATCAGCTGAGGCAAG |
| | | GAGACAACTTTGAAGTGTGGGAGAGACCCCTGAGCG |
| | | GCCTGGCCTGGGCTGTGGCCATGATCAACAGGCAAGA |
| | | GATTGGCGGCCCTAGAAGCTACACCATTGCTGTGGCT |
| | | AGCCTGGGCAAGGGAGTGGCCTGCAACCCTGCCTGCT |
| | | TCATCACACAGCTGCTGCCTGTGAAGAGGAAGCTGGG |
| | | CTTCTATGAGTGGACAAGCAGGCTGAGATCACACATC |
| | | AACCCCACCGGCACTGTGCTGCTGCAGCTGGAGAACA |
| | | CCATGCAGATGAGCCTGAAGGACCTGCTGTGAGCGGC |
| | | CGCCTGTGCCTTCTAGTTGCCAGCCATCTGTTGTTTGCCC |
| | | CTCCCCCGTGCCTTCCTTGACCCTGGAAGGTGCCACTCC |
| | | CACTGTCCTTTCCTAATAAAATGAGGAAATTGCATCGCATT |
| | | GTCTGAGTAGGTGTCATTCTATTCTGGGGGGTGGGGTGG |
| | | GGCAGGACAGCAAGGGGGAGGATTGGGAAGACAATAGC |
| | | AGGCATGCTGGGGA |
| 18 | Nucleotide sequence of human α- | GGCAAAGTCCATATTGACTTAGGTTAATCATTAACTCT |
| | galactosidase A expression | TTGTTGACTAAGTCAATAATCCTCGAGTGCCCATGTAA |
| | cassette containing promoter | GGAGGCAAGGCCTGGGGACACCCGAGATGCCTGGTTA |
| | HC7, hGLAco1 and bovine | TAATTAACCCAGACATGTGGCTGCCCCCCCCCCCCCAA |
| | growth hormone (bGH) poly A | CACCTGCTGCCTCTAAAAATAACCCTGCATGCCATGTT |
| | sequences (Cassette 3) | CCCGGCGAAGGGCCAGCTGTCCCCCGCGGGCAGCCCA |
| | | TACAAGGCCATGGGGCTGGGCAAGCTGCACGCCAGGG |
| | | GCCCCTCCCTGGGGACAGCCCCTCCTGGCTAGTCACAC |
| | | CCTGTAGGCTCCTCTATATAACCCAGGGGCACAGGGG |
| | | CTGCCCTCATTCTACCACCACCTCCACAGCACAGACAG |
| | | ACACTCAGGAGCCAGCCAGCGGATCCGCCACCATGCA |
| | | GCTGAGGAACCCTGAGCTGCACCTGGGCTGTGCCCTG |
| | | GCCCTGAGGTTCCTGGCCCTGGTGAGCTGGGACATCC |
| | | CCGGGGCTAGAGCCCTGGACAATGGCCTGGCTAGGA |
| | | CCCCCACCATGGGCTGGCTGCACTGGGAGAGGTTCAT |
| | | GTGCAACCTGGACTGCCAAGAGGAGCCTGACAGCTG |
| | | CATCAGTGAGAAGCTGTTCATGGAGATGGCTGAGCTG |
| | | ATGGTGTCAGAGGGCTGGAAGGATGCCGGCTATGAG |
| | | TACCTGTGCATTGATGACTGCTGGATGGCCCCTCAGA |
| | | GAGACTCAGAAGGCAGACTGCAAGCTGACCCTCAGA |
| | | GGTTCCCCCATGGCATCAGGCAGCTGGCCAACTATGT |
| | | GCACAGCAAGGGCCTGAAGCTGGGCATCTATGCAGA |
| | | TGTGGGCAACAAGACCTGTGCCGGCTTCCCCGGCAGC |
| | | TTTGGCTACTATGACATTGATGCCCAGACCTTTGCTGA |
| | | CTGGGGAGTGGACCTGCTGAAGTTTGATGGCTGCTAC |
| | | TGTGACAGCCTGGAGAACCTGGCAGATGGCTACAAG |
| | | CACATGAGCCTGGCCCTGAACAGGACCGGCAGAAGC |
| | | ATTGTGTACAGCTGTGAGTGGCCCCTGTACATGTGGC |
| | | CCTTTCAGAAGCCCAACTACACAGAGATCAGACAGTA |
| | | CTGCAACCACTGGAGGAACTTTGCTGACATTGATGAC |
| | | AGCTGGAAGAGCATCAAGAGCATCCTGGACTGGACA |
| | | AGCTTCAACCAAGAGAGGATTGTGGATGTGGCCGGCC |
| | | CCGGCGGCTGGAATGACCCTGACATGCTGGTGATTGG |
| | | CAACTTTGGCCTGAGCTGGAATCAGCAAGTGACACAG |
| | | ATGGCCCTGTGGGCCATCATGGCTGCCCCCCTGTTCA |
| | | TGAGCAATGACCTGAGGCACATCAGCCCCCAAGCCA |
| | | AGGCCCTGCTGCAAGACAAGGATGTGATTGCCATCAA |
| | | CCAAGACCCCCTGGGCAAGCAAGGCTATCAGCTGAG |
| | | GCAAGGAGACAACTTTGAAGTGTGGGAGAGACCCCT |
| | | GAGCGGCCTGGCCTGGGCTGTGGCCATGATCAACAGG |
| | | CAAGAGATTGGCGGCCCTAGAAGCTACACCATTGCTG |
| | | TGGCTAGCCTGGGCAAGGGAGTGGCCTGCAACCCTGC |
| | | CTGCTTCATCACACAGCTGCTGCCTGTGAAGAGGAAG |
| | | CTGGGCTTCTATGAGTGGACAAGCAGGCTGAGATCAC |
| | | ACATCAACCCCACCGGCACTGTGCTGCTGCAGCTGGA |
| | | GAACACCATGCAGATGAGCCTGAAGGACCTGCTGTG |
| | | AGCGGCCGCCTGTGCCTTCTAGTTGCCAGCCATCTGTTG |
| | | TTTGCCCCTCCCCCGTGCCTTCCTTGACCCTGGAAGGTG |
| | | CCACTCCCACTGTCCTTTCCTAATAAAATGAGGAAATTGC |
| | | ATCGCATTGTCTGAGTAGGTGTCATTCTATTCTGGGGGGT |
| | | GGGGTGGGGCAGGACAGCAAGGGGGAGGATTGGGAAG |
| | | ACAATAGCAGGCATGCTGGGGA |
| 19 | Nucleotide sequence of truncated | GGCAAAGTCCATATTGACTTAGGTTAATCATTAACTCT |
| | human α-galactosidase A | TTGTTGACTAAGTCAATAATCCTCGAGTGCCCATGTAA |
| | expression cassette containing | GGAGGCAAGGCCTGGGGACACCCGAGATGCCTGGTTA |
| | promoter HC7, thGLAco1 and | TAATTAACCCAGACATGTGGCTGCCCCCCCCCCCCCAA |
| | bovine growth hormone (bGH) | CACCTGCTGCCTCTAAAAATAACCCTGCATGCCATGTT |
| | poly A sequences (Cassette 4) | CCCGGCGAAGGGCCAGCTGTCCCCCGCGGGCAGCCCA |
| | | TACAAGGCCATGGGGCTGGGCAAGCTGCACGCCAGGG |
| | | GCCCCTCCCTGGGGACAGCCCCTCCTGGCTAGTCACAC |
| | | CCTGTAGGCTCCTCTATATAACCCAGGGGCACAGGGG |
| | | CTGCCCTCATTCTACCACCACCTCCACAGCACAGACAG |
| | | ACACTCAGGAGCCAGCCAGCGGATCCGCCACCATGCA |
| | | GCTGAGGAACCCTGAGCTGCACCTGGGCTGTGCCCTG |
| | | GCCCTGAGGTTCCTGGCCCTGGTGAGCTGGGACATCC |
| | | CCGGGGCTAGAGCCCTGGACAATGGCCTGGCTAGGA |
| | | CCCCCACCATGGGCTGGCTGCACTGGGAGAGGTTCAT |
| | | GTGCAACCTGGACTGCCAAGAGGAGCCTGACAGCTG |
| | | CATCAGTGAGAAGCTGTTCATGGAGATGGCTGAGCTG |
| | | ATGGTGTCAGAGGGCTGGAAGGATGCCGGCTATGAG |
| | | TACCTGTGCATTGATGACTGCTGGATGGCCCCTCAGA |
| | | GAGACTCAGAAGGCAGACTGCAAGCTGACCCTCAGA |
| | | GGTTCCCCCATGGCATCAGGCAGCTGGCCAACTATGT |
| | | GCACAGCAAGGGCCTGAAGCTGGGCATCTATGCAGA |
| | | TGTGGGCAACAAGACCTGTGCCGGCTTCCCCGGCAGC |
| | | TTTGGCTACTATGACATTGATGCCCAGACCTTTGCTGA |
| | | CTGGGGAGTGGACCTGCTGAAGTTTGATGGCTGCTAC |
| | | TGTGACAGCCTGGAGAACCTGGCAGATGGCTACAAG |
| | | CACATGAGCCTGGCCCTGAACAGGACCGGCAGAAGC |
| | | ATTGTGTACAGCTGTGAGTGGCCCCTGTACATGTGGC |
| | | CCTTTCAGAAGCCCAACTACACAGAGATCAGACAGTA |
| | | CTGCAACCACTGGAGGAACTTTGCTGACATTGATGAC |
| | | AGCTGGAAGAGCATCAAGAGCATCCTGGACTGGACA |
| | | AGCTTCAACCAAGAGAGGATTGTGGATGTGGCCGGCC |
| | | CCGGCGGCTGGAATGACCCTGACATGCTGGTGATTGG |
| | | CAACTTTGGCCTGAGCTGGAATCAGCAAGTGACACAG |
| | | ATGGCCCTGTGGGCCATCATGGCTGCCCCCCTGTTCA |
| | | TGAGCAATGACCTGAGGCACATCAGCCCCCAAGCCA |
| | | AGGCCCTGCTGCAAGACAAGGATGTGATTGCCATCAA |
| | | CCAAGACCCCCTGGGCAAGCAAGGCTATCAGCTGAG |
| | | GCAAGGAGACAACTTTGAAGTGTGGGAGAGACCCCT |
| | | GAGCGGCCTGGCCTGGGCTGTGGCCATGATCAACAGG |
| | | CAAGAGATTGGCGGCCCTAGAAGCTACACCATTGCTG |
| | | TGGCTAGCCTGGGCAAGGGAGTGGCCTGCAACCCTGC |
| | | CTGCTTCATCACACAGCTGCTGCCTGTGAAGAGGAAG |
| | | CTGGGCTTCTATGAGTGGACAAGCAGGCTGAGATCAC |
| | | ACATCAACCCCACCGGCACTGTGCTGCTGCAGCTGGA |
| | | GAACACCATGCAGATGAGCCTGAAGGACTGAGCGGC |
| | | CGCCTGTGCCTTCTAGTTGCCAGCCATCTGTTGTTTGCCC |
| | | CTCCCCCGTGCCTTCCTTGACCCTGGAAGGTGCCACTCC |
| | | CACTGTCCTTTCCTAATAAAATGAGGAAATTGCATCGCATT |
| | | GTCTGAGTAGGTGTCATTCTATTCTGGGGGGTGGGGTGG |
| | | GGCAGGACAGCAAGGGGGAGGATTGGGAAGACAATAGC |
| | | AGGCATGCTGGGGA |
| 20 | Nucleotide sequence of codon- | ATGCAGCTGAGGAACCCTGAGCTGCACCTGGGCTGTG |
| | optimized human α-galactosidase | CCCTGGCCCTGAGGTTCCTGGCCCTGGTGAGCTGGGA |
| | A with 2 amino acids truncation at | CATCCCCGGGGCTAGAGCCCTGGACAATGGCCTGGCT |
| | C-terminus (thGLAco1) | AGGACCCCCACCATGGGCTGGCTGCACTGGGAGAGG |
| | | TTCATGTGCAACCTGGACTGCCAAGAGGAGCCTGACA |
| | | GCTGCATCAGTGAGAAGCTGTTCATGGAGATGGCTGA |
| | | GCTGATGGTGTCAGAGGGCTGGAAGGATGCCGGCTAT |
| | | GAGTACCTGTGCATTGATGACTGCTGGATGGCCCCTC |
| | | AGAGAGACTCAGAAGGCAGACTGCAAGCTGACCCTC |
| | | AGAGGTTCCCCCATGGCATCAGGCAGCTGGCCAACTA |
| | | TGTGCACAGCAAGGGCCTGAAGCTGGGCATCTATGCA |
| | | GATGTGGGCAACAAGACCTGTGCCGGCTTCCCCGGCA |
| | | GCTTTGGCTACTATGACATTGATGCCCAGACCTTTGCT |
| | | GACTGGGGAGTGGACCTGCTGAAGTTTGATGGCTGCT |
| | | ACTGTGACAGCCTGGAGAACCTGGCAGATGGCTACA |
| | | AGCACATGAGCCTGGCCCTGAACAGGACCGGCAGAA |
| | | GCATTGTGTACAGCTGTGAGTGGCCCCTGTACATGTG |
| | | GCCCTTTCAGAAGCCCAACTACACAGAGATCAGACAG |
| | | TACTGCAACCACTGGAGGAACTTTGCTGACATTGATG |
| | | ACAGCTGGAAGAGCATCAAGAGCATCCTGGACTGGA |
| | | CAAGCTTCAACCAAGAGAGGATTGTGGATGTGGCCG |
| | | GCCCCGGCGGCTGGAATGACCCTGACATGCTGGTGAT |
| | | TGGCAACTTTGGCCTGAGCTGGAATCAGCAAGTGACA |
| | | CAGATGGCCCTGTGGGCCATCATGGCTGCCCCCCTGT |
| | | TCATGAGCAATGACCTGAGGCACATCAGCCCCCAAGC |
| | | CAAGGCCCTGCTGCAAGACAAGGATGTGATTGCCATC |
| | | AACCAAGACCCCCTGGGCAAGCAAGGCTATCAGCTG |
| | | AGGCAAGGAGACAACTTTGAAGTGTGGGAGAGACCC |
| | | CTGAGCGGCCTGGCCTGGGCTGTGGCCATGATCAACA |
| | | GGCAAGAGATTGGCGGCCCTAGAAGCTACACCATTGC |
| | | TGTGGCTAGCCTGGGCAAGGGAGTGGCCTGCAACCCT |
| | | GCCTGCTTCATCACACAGCTGCTGCCTGTGAAGAGGA |
| | | AGCTGGGCTTCTATGAGTGGACAAGCAGGCTGAGATC |
| | | ACACATCAACCCCACCGGCACTGTGCTGCTGCAGCTG |
| | | GAGAACACCATGCAGATGAGCCTGAAGGACTGA |
| 21 | wild-type AAV9 VP1 amino acid | MAADGYLPDWLEDNLSEGIREWWALKPGAPQPKANQQ |
| | sequence | HQDNARGLVLPGYKYLGPGNGLDKGEPVNAADAAALE |
| | (underlined: position 586-588) | HDKAYDQQLKAGDNPYLKYNHADAEFQERLKEDTSFG |
| | (Uniprot Q6JC40_9VIRU) | GNLGRAVFQAKKRLLEPLGLVEEAAKTAPGKKRPVEQS |
| | VP1: amino acids 1-736 | PQEPDSSAGIGKSGAQPAKKRLNFGQTGDTESVPDPQPI |
| | VP2: amino acids 138-736 | GEPPAAPSGVGSLTMASGGGAPVADNNEGADGVGSSSG |
| | VP3: amino acids 203-736 | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSAQAQAQTGWVQNQGILPGMV |
| | | WQDRDVYLQGPIWAKIPHTDGNFHPSPLMGGFGMKHP |
| | | PPQILIKNTPVPADPPTAFNKDKLNSFITQYSTGQVSVEIE |
| | | WELQKENSKRWNPEIQYTSNYYKSNNVEFAVNTEGVY |
| | | SEPRPIGTRYLTRNL |
| 22 | wild-type AAV9 VP2 amino acid | TAPGKKRPVEQSPQEPDSSAGIGKSGAQPAKKRLNFGQT |
| | sequence | GDTESVPDPQPIGEPPAAPSGVGSLTMASGGGAPVADN |
| | | NEGADGVGSSSGNWHCDSQWLGDRVITTSTRTWALPT |
| | | YNNHLYKQISNSTSGGSSNDNAYFGYSTPWGYFDFNRF |
| | | HCHFSPRDWQRLINNNWGFRPKRLNFKLFNIQVKEVTD |
| | | NNGVKTIANNLTSTVQVFTDSDYQLPYVLGSAHEGCLP |
| | | PFPADVFMIPQYGYLTLNDGSQAVGRSSFYCLEYFPSQM |
| | | LRTGNNFQFSYEFENVPFHSSYAHSQSLDRLMNPLIDQY |
| | | LYYLSKTINGSGQNQQTLKFSVAGPSNMAVQGRNYIPG |
| | | PSYRQQRVSTTVTQNNNSEFAWPGASSWALNGRNSLM |
| | | NPGPAMASHKEGEDRFFPLSGSLIFGKQGTGRDNVDAD |
| | | KVMITNEEEIKTTNPVATESYGQVATNHQSAQAQAQTG |
| | | WVQNQGILPGMVWQDRDVYLQGPIWAKIPHTDGNFHP |
| | | SPLMGGFGMKHPPPQILIKNTPVPADPPTAFNKDKLNSFI |
| | | TQYSTGQVSVEIEWELQKENSKRWNPEIQYTSNYYKSN |
| | | NVEFAVNTEGVYSEPRPIGTRYLTRNL |
| 23 | wild-type AAV9 VP3 amino acid | MASGGGAPVADNNEGADGVGSSSGNWHCDSQWLGDR |
| | sequence | VITTSTRTWALPTYNNHLYKQISNSTSGGSSNDNAYFGY |
| | | STPWGYFDFNRFHCHFSPRDWQRLINNNWGFRPKRLNF |
| | | KLFNIQVKEVTDNNGVKTIANNLTSTVQVFTDSDYQLP |
| | | YVLGSAHEGCLPPFPADVFMIPQYGYLTLNDGSQAVGR |
| | | SSFYCLEYFPSQMLRTGNNFQFSYEFENVPFHSSYAHSQ |
| | | SLDRLMNPLIDQYLYYLSKTINGSGQNQQTLKFSVAGPS |
| | | NMAVQGRNYIPGPSYRQQRVSTTVTQNNNSEFAWPGAS |
| | | SWALNGRNSLMNPGPAMASHKEGEDRFFPLSGSLIFGK |
| | | QGTGRDNVDADKVMITNEEEIKTTNPVATESYGQVATN |
| | | HQSAQAQAQTGWVQNQGILPGMVWQDRDVYLQGPIW |
| | | AKIPHTDGNFHPSPLMGGFGMKHPPPQILIKNTPVPADPP |
| | | TAFNKDKLNSFITQYSTGQVSVEIEWELQKENSKRWNPE |
| | | IQYTSNYYKSNNVEFAVNTEGVYSEPRPIGTRYLTRNL |
| 24 | AVT901 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic: insert | PQEPDSSAGIGKSGAQPAKKRLNFGQTGDTESVPDPQPI |
| | after wild-type position 453) | GEPPAAPSGVGSLTMASGGGAPVADNNEGADGVGSSSG |
| | | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGHMN |
| | | GRNNSGQNQQTLKFSVAGPSNMAVQGRNYIPGPSYRQQ |
| | | RVSTTVTQNNNSEFAWPGASSWALNGRNSLMNPGPAM |
| | | ASHKEGEDRFFPLSGSLIFGKQGTGRDNVDADKVMITNE |
| | | EEIKTTNPVATESYGQVATNHQSAQAQAQTGWVQNQGI |
| | | LPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 25 | AVT901 VP1's insert peptide | HMNGRNN |
| | amino acid sequence | |
| 26 | AVT902 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic: insert | PQEPDSSAGIGKSGAQPAKKRLNFGQTGDTESVPDPQPI |
| | after wild-type position 453) | GEPPAAPSGVGSLTMASGGGAPVADNNEGADGVGSSSG |
| | | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGPHY |
| | | THGISGQNQQTLKFSVAGPSNMAVQGRNYIPGPSYRQQ |
| | | RVSTTVTQNNNSEFAWPGASSWALNGRNSLMNPGPAM |
| | | ASHKEGEDRFFPLSGSLIFGKQGTGRDNVDADKVMITNE |
| | | EEIKTTNPVATESYGQVATNHQSAQAQAQTGWVQNQGI |
| | | LPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 27 | AVT902 VP1's insert peptide | PHYTHGI |
| | amino acid sequence | |
| 28 | AVT903 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSAQRGDTNNTNMAQAQTGWVQN |
| | | QGILPGMVWQDRDVYLQGPIWAKIPHTDGNFHPSPLMG |
| | | GFGMKHPPPQILIKNTPVPADPPTAFNKDKLNSFITQYST |
| | | GQVSVEIEWELQKENSKRWNPEIQYTSNYYKSNNVEFA |
| | | VNTEGVYSEPRPIGTRYLTRNL |
| 29 | AVT903 VP1's insert peptide | RGDTNNTNM |
| | amino acid sequence | |
| 30 | AVT905 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSAQSRGDRPMAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 31 | AVT905 VP1's insert peptide | SRGDRPM |
| | amino acid sequence | |
| 32 | AVT906 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSAQLHKGIDIAQAQTGWVQNQGI |
| | | LPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 33 | AVT906 VP1's insert peptide | LHKGIDI |
| | amino acid sequence | |
| 34 | AVT907 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic: insert | PQEPDSSAGIGKSGAQPAKKRLNFGQTGDTESVPDPQPI |
| | after wild-type position 453; italic | GEPPAAPSGVGSLTMASGGGAPVADNNEGADGVGSSSG |
| | and underlined: insert after wild- | NWHCDSQWLGDRVITTSTRTWALPTYNNHLYKQISNST |
| | type position 588) | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGHMN |
| | | GRNNSGQNQQTLKFSVAGPSNMAVQGRNYIPGPSYRQQ |
| | | RVSTTVTQNNNSEFAWPGASSWALNGRNSLMNPGPAM |
| | | ASHKEGEDRFFPLSGSLIFGKQGTGRDNVDADKVMITNE |
| | | EEIKTTNPVATESYGQVATNHQGPGRGDQTTLAQAQTG |
| | | WVQNQGILPGMVWQDRDVYLQGPIWAKIPHTDGNFHP |
| | | SPLMGGFGMKHPPPQILIKNTPVPADPPTAFNKDKLNSFI |
| | | TQYSTGQVSVEIEWELQKENSKRWNPEIQYTSNYYKSN |
| | | NVEFAVNTEGVYSEPRPIGTRYLTRNL |
| 35 | AVT907 VP1's insert peptide | HMNGRNN |
| | (after wild-type position 453) | |
| | amino acid sequence | |
| 36 | AVT907 VP1's amino acid | GPGRGDQTTL |
| | sequence corresponding to | |
| | position 586-588 of wild-type | |
| | AAV9 VP1, plus insert peptide | |
| | (after wild-type position 588) | |
| 37 | AVT908 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSAQSRGDRPMAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 38 | AVT908 VP1's insert peptide | SRGDRPM |
| | amino acid sequence | |
| 39 | AVT909 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSAQGVNGRSTAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 40 | AVT909 VP1's insert peptide | GVNGRST |
| | amino acid sequence | |
| 41 | AVT910 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSAQGVNGRQVAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 42 | AVT910 VP1's insert peptide | GVNGRQV |
| | amino acid sequence | |
| 43 | AVT911 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic: insert | PQEPDSSAGIGKSGAQPAKKRLNFGQTGDTESVPDPQPI |
| | after wild-type position 453) | GEPPAAPSGVGSLTMASGGGAPVADNNEGADGVGSSSG |
| | | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGQTY |
| | | THNKSGQNQQTLKFSVAGPSNMAVQGRNYIPGPSYRQQ |
| | | RVSTTVTQNNNSEFAWPGASSWALNGRNSLMNPGPAM |
| | | ASHKEGEDRFFPLSGSLIFGKQGTGRDNVDADKVMITNE |
| | | EEIKTTNPVATESYGQVATNHQSAQAQAQTGWVQNQGI |
| | | LPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 44 | AVT911 VP1's insert peptide | QTYTHNK |
| | amino acid sequence | |
| 45 | AVT912 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic: insert | PQEPDSSAGIGKSGAQPAKKRLNFGQTGDTESVPDPQPI |
| | after wild-type position 453; italic | GEPPAAPSGVGSLTMASGGGAPVADNNEGADGVGSSSG |
| | and underlined: insert after wild- | NWHCDSQWLGDRVITTSTRTWALPTYNNHLYKQISNST |
| | type position 588) | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGQTY |
| | | THNKSGQNQQTLKFSVAGPSNMAVQGRNYIPGPSYRQQ |
| | | RVSTTVTQNNNSEFAWPGASSWALNGRNSLMNPGPAM |
| | | ASHKEGEDRFFPLSGSLIFGKQGTGRDNVDADKVMITNE |
| | | EEIKTTNPVATESYGQVATNHQSAQSRGDRPMAQAQTG |
| | | WVQNQGILPGMVWQDRDVYLQGPIWAKIPHTDGNFHP |
| | | SPLMGGFGMKHPPPQILIKNTPVPADPPTAFNKDKLNSFI |
| | | TQYSTGQVSVEIEWELQKENSKRWNPEIQYTSNYYKSN |
| | | NVEFAVNTEGVYSEPRPIGTRYLTRNL |
| 46 | AVT912 VP1's insert peptide | QTYTHNK |
| | (after wild-type position 453) | |
| | amino acid sequence | |
| 47 | AVT912 VP1's insert peptide | SRGDRPM |
| | (after wild-type position 588) | |
| | amino acid sequence | |
| 48 | AVT913 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQENRRGDTNALAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 49 | AVT913 VP1's amino acid | ENRRGDTNAL |
| | sequence corresponding to | |
| | position 586-588 of wild-type | |
| | AAV9 VP1 plus insert peptide | |
| 50 | AVT914 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNSRGDHNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 51 | AVT914 VP1's amino acid | NNSRGDHNSL |
| | sequence corresponding to | |
| | position 586-588 of wild-type | |
| | AAV9 VP1 plus insert peptide | |
| 52 | AVT915 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNSRGDYNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 53 | AVT915 VP1's amino acid | NNSRGDYNSL |
| | sequence corresponding to | |
| | position 586-588 of wild-type | |
| | AAV9 VP1 plus insert peptide | |
| 54 | AVT916 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNLRGDHNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 55 | AVT916 VP1's amino acid | NNLRGDHNSL |
| | sequence corresponding to | |
| | position 586-588 of wild-type | |
| | AAV9 VP1 plus insert peptide | |
| 56 | AVT917 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNARGDHNSLAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 57 | AVT917 VP1's amino acid | NNARGDHNSL |
| | sequence corresponding to | |
| | position 586-588 of wild-type | |
| | AAV9 VP1 plus insert peptide | |
| 58 | AVT918 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQGNIRGDHNTLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 59 | AVT918 VP1's amino acid | GNIRGDHNTL |
| | sequence corresponding to | |
| | position 586-588 of wild-type | |
| | AAV9 VP1 plus insert peptide | |
| 60 | AVT919 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQMNVRGDTNKLAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 61 | AVT919 VP1's amino acid | MNVRGDTNKL |
| | sequence corresponding to | |
| | position 586-588 of wild-type | |
| | AAV9 VP1 plus insert peptide | |
| 62 | AVT920 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQTNMRGDHNALAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 63 | AVT920 VP1's amino acid | TNMRGDHNAL |
| | sequence corresponding to | |
| | position 586-588 of wild-type | |
| | AAV9 VP1 plus insert peptide | |
| 64 | AV9ML001 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNNRGDNNYLAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 65 | AV9ML001 VP1's amino acid | NNNRGDNNYL |
| | sequence corresponding to | |
| | position 586-588 of wild-type | |
| | AAV9 VP1 plus insert peptide | |
| 66 | AV9ML002 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNNRGDHNTLAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 67 | AV9ML002 VP1's amino acid | NNNRGDHNTL |
| | sequence corresponding to | |
| | position 586-588 of wild-type | |
| | AAV9 VP1 plus insert peptide | |
| 68 | AV9ML004 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNERGDRNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 69 | AV9ML004 VP1's amino acid | NNERGDRNSL |
| | sequence corresponding to | |
| | position 586-588 of wild-type | |
| | AAV9 VP1 plus insert peptide | |
| 70 | AV9ML005 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNSRGDHNTLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 71 | AV9ML005 VP1's amino acid | NNSRGDHNTL |
| | sequence corresponding to | |
| | position 586-588 of wild-type | |
| | AAV9 VP1 plus insert peptide | |
| 72 | AV9ML007 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQENKRGDHNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 73 | AV9ML007 VP1's amino acid | ENKRGDHNSL |
| | sequence corresponding to | |
| | position 586-588 of wild-type | |
| | AAV9 VP1 plus insert peptide | |
| 74 | AV9ML012 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQINSRGDHNVLAQAQTGWVQNQGI |
| | | LPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 75 | AV9ML012 VP1's amino acid | INSRGDHNVL |
| | sequence corresponding to | |
| | position 586-588 of wild-type | |
| | AAV9 VP1 plus insert peptide | |
| 76 | AV9ML014 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNNRGDYNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 77 | AV9ML014 VP1's amino acid | NNNRGDYNSL |
| | sequence corresponding to | |
| | position 586-588 of wild-type | |
| | AAV9 VP1 plus insert peptide | |
| 78 | AV9ML016 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSAQRGDKLSIAQAQTGWVQNQGI |
| | | LPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 79 | AV9ML016 VP1's insert peptide | RGDKLSI |
| | amino acid sequence | |
| 80 | AV9ML018 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSAQLNTNNSFTKAQAQTGWVQN |
| | | QGILPGMVWQDRDVYLQGPIWAKIPHTDGNFHPSPLMG |
| | | GFGMKHPPPQILIKNTPVPADPPTAFNKDKLNSFITQYST |
| | | GQVSVEIEWELQKENSKRWNPEIQYTSNYYKSNNVEFA |
| | | VNTEGVYSEPRPIGTRYLTRNL |
| 81 | AV9ML018 VP1's insert peptide | LNTNNSFTK |
| | amino acid sequence | |
| 82 | AV9ML025 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSNSRGDHNTLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 83 | AV9ML025 VP1's amino acid | SNSRGDHNTL |
| | sequence corresponding to | |
| | position 586-588 of wild-type | |
| | AAV9 VP1 plus insert peptide | |
| | insert peptide | |
| 84 | AV9ML026 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSNSRGDHNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 85 | AV9ML026 VP1's amino acid | SNSRGDHNSL |
| | sequence corresponding to | |
| | position 586-588 of wild-type | |
| | AAV9 VP1 plus insert peptide | |
| | insert peptide | |
| 86 | AV9ML027 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQENRRGDHNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 87 | AV9ML027 VP1's amino acid | ENRRGDHNSL |
| | sequence corresponding to | |
| | position 586-588 of wild-type | |
| | AAV9 VP1 plus insert peptide | |
| | insert peptide | |
| 88 | AV9ML028 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQGNSRGDYNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 89 | AV9ML028 VP1's amino acid | GNSRGDYNSL |
| | sequence corresponding to | |
| | position 586-588 of wild-type | |
| | AAV9 VP1 plus insert peptide | |
| | insert peptide | |
| 90 | AV9ML029 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNSRGDRNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 91 | AV9ML029 VP1's amino acid | NNSRGDRNSL |
| | sequence corresponding to | |
| | position 586-588 of wild-type | |
| | AAV9 VP1 plus insert peptide | |
| | insert peptide | |
| 92 | AV9ML030 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQMNVRGDTNSLAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 93 | AV9ML030 VP1's amino acid | MNVRGDTNSL |
| | sequence corresponding to | |
| | position 586-588 of wild-type | |
| | AAV9 VP1 plus insert peptide | |
| | insert peptide | |
| 94 | AV9ML031 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSNARGDHNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 95 | AV9ML031 VP1's amino acid | SNARGDHNSL |
| | sequence corresponding to | |
| | position 586-588 of wild-type | |
| | AAV9 VP1 plus insert peptide | |
| | insert peptide | |
| 96 | AV9ML032 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQGNSRGDHNTLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 97 | AV9ML032 VP1's amino acid | GNSRGDHNTL |
| | sequence corresponding to | |
| | position 586-588 of wild-type | |
| | AAV9 VP1 plus insert peptide | |
| | insert peptide | |
| 98 | AV9ML033 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNNRGDHNALAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 99 | AV9ML033 VP1's amino acid | NNNRGDHNAL |
| | sequence corresponding to | |
| | position 586-588 of wild-type | |
| | AAV9 VP1 plus insert peptide | |
| | insert peptide | |
| 100 | AV9ML034 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNLRGDRNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 101 | AV9ML034 VP1's amino acid | NNLRGDRNSL |
| | sequence corresponding to | |
| | position 586-588 of wild-type | |
| | AAV9 VP1 plus insert peptide | |
| | insert peptide | |
| 102 | AV9ML035 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQDNRRGDTNALAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 103 | AV9ML035 VP1's amino acid | DNRRGDTNAL |
| | sequence corresponding to | |
| | position 586-588 of wild-type | |
| | AAV9 VP1 plus insert peptide | |
| | insert peptide | |
| 104 | AV9ML036 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQENERGDHNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 105 | AV9ML036 VP1's amino acid | ENERGDHNSL |
| | sequence corresponding to | |
| | position 586-588 of wild-type | |
| | AAV9 VP1 plus insert peptide | |
| | insert peptide | |
| 106 | AV9ML037 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNARGDTNALAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 107 | AV9ML037 VP1's amino acid | NNARGDTNAL |
| | sequence corresponding to | |
| | position 586-588 of wild-type | |
| | AAV9 VP1 plus insert peptide | |
| | insert peptide | |
| 108 | AV9ML038 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQSNLRGDHNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 109 | AV9ML038 VP1's amino acid | SNLRGDHNSL |
| | sequence corresponding to | |
| | position 586-588 of wild-type | |
| | AAV9 VP1 plus insert peptide | |
| | insert peptide | |
| 110 | AV9ML039 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQMSVRGDTNKLAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 111 | AV9ML039 VP1's amino acid | MSVRGDTNKL |
| | sequence corresponding to | |
| | position 586-588 of wild-type | |
| | AAV9 VP1 plus insert peptide | |
| | insert peptide | |
| 112 | AV9ML040 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQMNVRGDTNKPAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 113 | AV9ML040 VP1's amino acid | MNVRGDTNKP |
| | sequence corresponding to | |
| | position 586-588 of wild-type | |
| | AAV9 VP1 plus insert peptide | |
| | insert peptide | |
| 114 | AV9ML041 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNLRGDTNALAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 115 | AV9ML041 VP1's amino acid | NNLRGDTNAL |
| | sequence corresponding to | |
| | position 586-588 of wild-type | |
| | AAV9 VP1 plus insert peptide | |
| | insert peptide | |
| 116 | AV9ML042 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQINSRGDYNVLAQAQTGWVQNQGI |
| | | LPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 117 | AV9ML042 VP1's amino acid | INSRGDYNVL |
| | sequence corresponding to | |
| | position 586-588 of wild-type | |
| | AAV9 VP1 plus insert peptide | |
| | insert peptide | |
| 118 | AV9ML043 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQKNRRGDTNALAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 119 | AV9ML043 VP1's amino acid | KNRRGDTNAL |
| | sequence corresponding to | |
| | position 586-588 of wild-type | |
| | AAV9 VP1 plus insert peptide | |
| | insert peptide | |
| 120 | AV9ML044 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQENERGDRNSLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 121 | AV9ML044 VP1's amino acid | ENERGDRNSL |
| | sequence corresponding to | |
| | position 586-588 of wild-type | |
| | AAV9 VP1 plus insert peptide | |
| | insert peptide | |
| 122 | AV9ML045 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQMNARGDTNKLAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 123 | AV9ML045 VP1's amino acid | MNARGDTNKL |
| | sequence corresponding to | |
| | position 586-588 of wild-type | |
| | AAV9 VP1 plus insert peptide | |
| | insert peptide | |
| 124 | AV9ML046 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQGNIRGDRNTLAQAQTGWVQNQGI |
| | | LPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 125 | AV9ML046 VP1's amino acid | GNIRGDRNTL |
| | sequence corresponding to | |
| | position 586-588 of wild-type | |
| | AAV9 VP1 plus insert peptide | |
| | insert peptide | |
| 126 | AV9ML047 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQGNIRGDHNALAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 127 | AV9ML047 VP1's amino acid | GNIRGDHNAL |
| | sequence corresponding to | |
| | position 586-588 of wild-type | |
| | AAV9 VP1 plus insert peptide | |
| | insert peptide | |
| 128 | AV9ML048 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQNNRRGDHNSLAQAQTGWVQNQ |
| | | GILPGMVWQDRDVYLQGPIWAKIPHTDGNFHPSPLMGG |
| | | FGMKHPPPQILIKNTPVPADPPTAFNKDKLNSFITQYSTG |
| | | QVSVEIEWELQKENSKRWNPEIQYTSNYYKSNNVEFAV |
| | | NTEGVYSEPRPIGTRYLTRNL |
| 129 | AV9ML048 VP1's amino acid | NNRRGDHNSL |
| | sequence corresponding to | |
| | position 586-588 of wild-type | |
| | AAV9 VP1 plus insert peptide | |
| | insert peptide | |
| 130 | AV9ML049 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQTNIRGDYNSLAQAQTGWVQNQGI |
| | | LPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 131 | AV9ML049 VP1's amino acid | TNIRGDYNSL |
| | sequence corresponding to | |
| | position 586-588 of wild-type | |
| | AAV9 VP1 plus insert peptide | |
| | insert peptide | |
| 132 | AV9ML050 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQRNLRGDHNTLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 133 | AV9ML050 VP1's amino acid | RNLRGDHNTL |
| | sequence corresponding to | |
| | position 586-588 of wild-type | |
| | AAV9 VP1 plus insert peptide | |
| | insert peptide | |
| 134 | AV9ML051 VP1 amino acid | MAADGYLPDWLEDNLSEGIREWWALKPGAPQPKANQQ |
| | sequence (underlined: | HQDNARGLVLPGYKYLGPGNGLDKGEPVNAADAAALE |
| | corresponding to position 586- | HDKAYDQQLKAGDNPYLKYNHADAEFQERLKEDTSFG |
| | 588 of wild-type AAV9 VP1 | GNLGRAVFQAKKRLLEPLGLVEEAAKTAPGKKRPVEQS |
| | amino acid sequence; italic and | PQEPDSSAGIGKSGAQPAKKRLNFGQTGDTESVPDPQPI |
| | underlined: insert after wild-type | GEPPAAPSGVGSLTMASGGGAPVADNNEGADGVGSSSG |
| | position 588) | NWHCDSQWLGDRVITTSTRTWALPTYNNHLYKQISNST |
| | | SGGSSNDNAYFGYSTPWGYFDFNRFHCHFSPRDWQRLI |
| | | NNNWGFRPKRLNFKLFNIQVKEVTDNNGVKTIANNLTS |
| | | TVQVFTDSDYQLPYVLGSAHEGCLPPFPADVFMIPQYG |
| | | YLTLNDGSQAVGRSSFYCLEYFPSQMLRTGNNFQFSYEF |
| | | ENVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGE |
| | | DRFFPLSGSLIFGKQGTGRDNVDADKVMITNEEEIKTTN |
| | | PVATESYGQVATNHQCNSRGDHNTLAQAQTGWVQNQG |
| | | ILPGMVWQDRDVYLQGPIWAKIPHTDGNFHPSPLMGGF |
| | | GMKHPPPQILIKNTPVPADPPTAFNKDKLNSFITQYSTGQ |
| | | VSVEIEWELQKENSKRWNPEIQYTSNYYKSNNVEFAVN |
| | | TEGVYSEPRPIGTRYLTRNL |
| 135 | AV9ML051 VP1's amino acid | CNSRGDHNTL |
| | sequence corresponding to | |
| | position 586-588 of wild-type | |
| | AAV9 VP1 plus insert peptide | |
| | insert peptide | |
| 136 | AAV2 (UniProt: P03135-1) | MAADGYLPDWLEDTLSEGIRQWWKLKPGPPPPKPAER |
| | VP1: amino acids 1-735 | HKDDSRGLVLPGYKYLGPFNGLDKGEPVNEADAAALE |
| | VP2: amino acids 138-735 | HDKAYDRQLDSGDNPYLKYNHADAEFQERLKEDTSFG |
| | VP3: amino acids 203-735 | GNLGRAVFQAKKRVLEPLGLVEEPVKTAPGKKRPVEHS |
| | | PVEPDSSSGTGKAGQQPARKRLNFGQTGDADSVPDPQP |
| | | LGQPPAAPSGLGTNTMATGSGAPMADNNEGADGVGNS |
| | | SGNWHCDSTWMGDRVITTSTRTWALPTYNNHLYKQISS |
| | | QSGASNDNHYFGYSTPWGYFDFNRFHCHFSPRDWQRLI |
| | | NNNWGFRPKRLNFKLFNIQVKEVTQNDGTTTIANNLTS |
| | | TVQVFTDSEYQLPYVLGSAHQGCLPPFPADVFMVPQYG |
| | | YLTLNNGSQAVGRSSFYCLEYFPSQMLRTGNNFTFSYTF |
| | | EDVPFHSSYAHSQSLDRLMNPLIDQYLYYLSRTNTPSGT |
| | | TTQSRLQFSQAGASDIRDQSRNWLPGPCYRQQRVSKTS |
| | | ADNNNSEYSWTGATKYHLNGRDSLVNPGPAMASHKDD |
| | | EEKFFPQSGVLIFGKQGSEKTNVDIEKVMITDEEEIRTTN |
| | | PVATEQYGSVSTNLQRGNRQAATADVNTQGVLPGMV |
| | | WQDRDVYLQGPIWAKIPHTDGHFHPSPLMGGFGLKHPP |
| | | PQILIKNTPVPANPSTTFSAAKFASFITQYSTGQVSVEIEW |
| | | ELQKENSKRWNPEIQYTSNYNKSVNVDFTVDINGVYSE |
| | | PRPIGTRYLTRNL |
| 137 | AAV8 | MAADGYLPDWLEDNLSEGIREWWALKPGAPKPKANQQ |
| | (Uniprot Q8JQF8_9VIRU) | KQDDGRGLVLPGYKYLGPFNGLDKGEPVNAADAAALE |
| | VP1: amino acids 1-738 | HDKAYDQQLQAGDNPYLRYNHADAEFQERLQEDTSFG |
| | VP2: amino acids 138-738 | GNLGRAVFQAKKRVLEPLGLVEEGAKTAPGKKRPVEPS |
| | VP3: amino acids 204-738 | PQRSPDSSTGIGKKGQQPARKRLNFGQTGDSESVPDPQP |
| | | LGEPPAAPSGVGPNTMAAGGGAPMADNNEGADGVGSS |
| | | SGNWHCDSTWLGDRVITTSTRTWALPTYNNHLYKQISN |
| | | GTSGGATNDNTYFGYSTPWGYFDFNRFHCHFSPRDWQ |
| | | RLINNNWGFRPKRLSFKLFNIQVKEVTQNEGTKTIANNL |
| | | TSTIQVFTDSEYQLPYVLGSAHQGCLPPFPADVFMIPQY |
| | | GYLTLNNGSQAVGRSSFYCLEYFPSQMLRTGNNFQFTY |
| | | TFEDVPFHSSYAHSQSLDRLMNPLIDQYLYYLSRTQTTG |
| | | GTANTQTLGFSQGGPNTMANQAKNWLPGPCYRQQRVS |
| | | TTTGQNNNSNFAWTAGTKYHLNGRNSLANPGIAMATH |
| | | KDDEERFFPSNGILIFGKQNAARDNADYSDVMLTSEEEI |
| | | KTTNPVATEEYGIVADNLQQQNTAPQIGTVNSQGALPG |
| | | MVWQNRDVYLQGPIWAKIPHTDGNFHPSPLMGGFGLK |
| | | HPPPQILIKNTPVPADPPTTFNQSKLNSFITQYSTGQVSVE |
| | | IEWELQKENSKRWNPEIQYTSNYYKSTSVDFAVNTEGV |
| | | YSEPRPIGTRYLTRNL |
| 138 | AAVrh8 | MAADGYLPDWLEDNLSEGIREWWDLKPGAPKPKANQQ |
| | (Uniprot Q808Y3_9VIRU) | KQDDGRGLVLPGYKYLGPFNGLDKGEPVNAADAAALE |
| | VP1: amino acids 1-736 | HDKAYDQQLKAGDNPYLRYNHADAEFQERLQEDTSFG |
| | VP2: amino acids 138-736 | GNLGRAVFQAKKRVLEPLGLVEEGAKTAPGKKRPVEQS |
| | VP3: amino acids 203-736 | PQEPDSSSGIGKTGQQPAKKRLNFGQTGDSESVPDPQPL |
| | | GEPPAAPSGLGPNTMASGGGAPMADNNEGADGVGNSS |
| | | GNWHCDSTWLGDRVITTSTRTWALPTYNNHLYKQISNG |
| | | TSGGSTNDNTYFGYSTPWGYFDFNRFHCHFSPRDWQRL |
| | | INNNWGFRPKRLNFKLFNIQVKEVTTNEGTKTIANNLTS |
| | | TVQVFTDSEYQLPYVLGSAHQGCLPPFPADVFMVPQYG |
| | | YLTLNNGSQALGRSSFYCLEYFPSQMLRTGNNFQFSYTF |
| | | EDVPFHSSYAHSQSLDRLMNPLIDQYLYYLVRTQTTGT |
| | | GGTQTLAFSQAGPSSMANQARNWVPGPCYRQQRVSTT |
| | | TNQNNNSNFAWTGAAKFKLNGRDSLMNPGVAMASHK |
| | | DDDDRFFPSSGVLIFGKQGAGNDGVDYSQVLITDEEEIK |
| | | ATNPVATEEYGAVAINNQAANTQAQTGLVHNQGVIPG |
| | | MVWQNRDVYLQGPIWAKIPHTDGNFHPSPLMGGFGLK |
| | | HPPPQILIKNTPVPADPPLTFNQAKLNSFITQYSTGQVSV |
| | | EIEWELQKENSKRWNPEIQYTSNYYKSTNVDFAVNTEG |
| | | VYSEPRPIGTRYLTRNL |
| 139 | AAVrh10 | MAADGYLPDWLEDNLSEGIREWWDLKPGAPKPKANQQ |
| | (Uniprot Q808W5_9VIRU) | KQDDGRGLVLPGYKYLGPFNGLDKGEPVNAADAAALE |
| | VP1: amino acids 1-738 | HDKAYDQQLKAGDNPYLRYNHADAEFQERLQEDTSFG |
| | VP2: amino acids 138-738 | GNLGRAVFQAKKRVLEPLGLVEEGAKTAPGKKRPVEPS |
| | VP3: amino acids 204-738 | PQRSPDSSTGIGKKGQQPAKKRLNFGQTGDSESVPDPQP |
| | | IGEPPAGPSGLGSGTMAAGGGAPMADNNEGADGVGSSS |
| | | GNWHCDSTWLGDRVITTSTRTWALPTYNNHLYKQISNG |
| | | TSGGSTNDNTYFGYSTPWGYFDFNRFHCHFSPRDWQRL |
| | | INNNWGFRPKRLNFKLFNIQVKEVTQNEGTKTIANNLTS |
| | | TIQVFTDSEYQLPYVLGSAHQGCLPPFPADVFMIPQYGY |
| | | LTLNNGSQAVGRSSFYCLEYFPSQMLRTGNNFEFSYQFE |
| | | DVPFHSSYAHSQSLDRLMNPLIDQYLYYLSRTQSTGGTA |
| | | GTQQLLFSQAGPNNMSAQAKNWLPGPCYRQQRVSTTL |
| | | SQNNNSNFAWTGATKYHLNGRDSLVNPGVAMATHKD |
| | | DEERFFPSSGVLMFGKQGAGKDNVDYSSVMLTSEEEIK |
| | | TTNPVATEQYGVVADNLQQQNAAPIVGAVNSQGALPG |
| | | MVWQNRDVYLQGPIWAKIPHTDGNFHPSPLMGGFGLK |
| | | HPPPQILIKNTPVPADPPTTFSQAKLASFITQYSTGQVSVE |
| | | IEWELQKENSKRWNPEIQYTSNYYKSTNVDFAVNTDGT |
| | | YSEPRPIGTRYLTRNL |
| 140 | AAV2v | MAADGYLPDWLEDTLSEGIRQWWKLKPGPPPPKPAER |
| | VP1: amino acids 1-735 | HKDDSRGLVLPGYKYLGPFNGLDKGEPVNEADAAALE |
| | VP2: amino acids 138-735; | HDKAYDRQLDSGDNPYLKYNHADAEFQERLKEDTSFG |
| | VP3: amino acids 204-735 | GNLGRAVFQAKKRVLEPLGLVEEPVKTAPGKKRPVEHS |
| | | PVEPDSSSGTGKAGQQPARKRLNFGQTGDADSVPDPQP |
| | | LGQPPAAPSGLGTNTMATGSGAPMADNNEGADGVGNS |
| | | SGNWHCDSTWMGDRVITTSTRTWALPTYNNHLYKQISS |
| | | QSGASNDNHYFGYSTPWGYFDFNRFHCHFSPRDWQRLI |
| | | NNNWGFRPKRLNFKLFNIQVKEVTQNDGTTTIANNLTS |
| | | TVQVFTDSEYQLPYVLGSAHQGCLPPFPADVFMVPQYG |
| | | YLTLNNGSQAVGRSSFYCLEYFPSQMLRTGNNFTFSYTF |
| | | EDVPFHSSYAHSQSLDRLMNPLIDQYLYFLSRTNTPSGT |
| | | TTQSRLQFSQAGASDIRDQSRNWLPGPCYRQQGVSKVS |
| | | ADNNNSEFSWTGATKYHLNGRDSLVNPGPAMASHKDD |
| | | EEKFFPQSGVLIFGKQGSEKTNVDIEKVMITDEEEIRTTN |
| | | PVATEQYGSVSTNLQSGNTQAATADVNTQGVLPGMVW |
| | | QDRDVYLQGPIWAKIPHTDGHFHPSPLMGGFGLKHPPP |
| | | QILIKNTPVPANPSTTFSAAKFASFITQYSTGQVSVEIEWE |
| | | LQKENSKRWNPEIQYTSNYNKSVNVDFTVDTNGVYSEP |
| | | RPIGTRFLTRNL |
| 141 | AAV44-9 | MAADGYLPDWLEDNLSEGIREWWDLKPGAPKPKANQQ |
| | VP1: amino acids 1-736 | KQDDGRGLVLPGYKYLGPFNGLDKGEPVNAADAAALE |
| | VP2: amino acids 138-736; | HDKAYDQQLKAGDNPYLRYNHADAEFQERLQEDTSFG |
| | VP3: amino acids 204-736 | GNLGRAVFQAKKRVLEPLGLVEEGAKTAPGKKRPVEQS |
| | | PQEPDSSSGIGKTGQQPAKKRLNFGQTGDTESVPDPQPL |
| | | GEPPAAPSGLGPNTMASGGGAPMADNNEGADGVGNSS |
| | | GNWHCDSTWLGDRVITTSTRTWALPTYNNHLYKQISNG |
| | | TSGGSTNDNTYFGYSTPWGYFDFNRFHCHFSPRDWQRL |
| | | INNNWGFRPKRLNFKLFNIQVKEVTTNEGTKTIANNLTS |
| | | TVQVFTDSEYQLPYVLGSAHQGCLPPFPADVFMVPQYG |
| | | YLTLNNGSQALGRSSFYCLEYFPSQMLRTGNNFQFSYTF |
| | | EDVPFHSSYAHSQSLDRLMNPLIDQYLYYLVRTQTTGT |
| | | GGTQTLAFSQAGPSNMASQARNWVPGPSYRQQRVSTTT |
| | | NQNNNSNFAWTGAAKFKLNGRDSLMNPGVAMASHKD |
| | | DEDRFFPSSGVLIFGKQGAGNDGVDYSQVLITDEEEIKA |
| | | TNPVATEEYGAVAINNQAANTQAQTGLVHNQGVIPGM |
| | | VWQNRDVYLQGPIWAKIPHTDGNFHPSPLMGGFGLKHP |
| | | PPQILIKNTPVPADPPLTFNQAKLNSFITQYSTGQVSVEIE |
| | | WELQKENSKRWNPEIQYTSNYYKSTNVDFAVNTEGVY |
| | | SEPRPIGTRYLTRNL |

### EXAMPLES

Certain embodiments provided herein are illustrated by the following non-limiting examples, which describe the designing and testing of various human α-galactosidase A encoding sequences, promoters, expression cassettes and recombinant adeno-associated virus (AAV) particles, showing that they are particularly suitable for gene therapies, especially for lysosomal storage disorders including Fabry disease.

### Example 1: Correction of Substrate Level in the Mouse Model of Fabry Disease by Intravenous Administration of AAV-GLA

### 1. Human α-Galactosidase a (GLA) Codon Optimization.

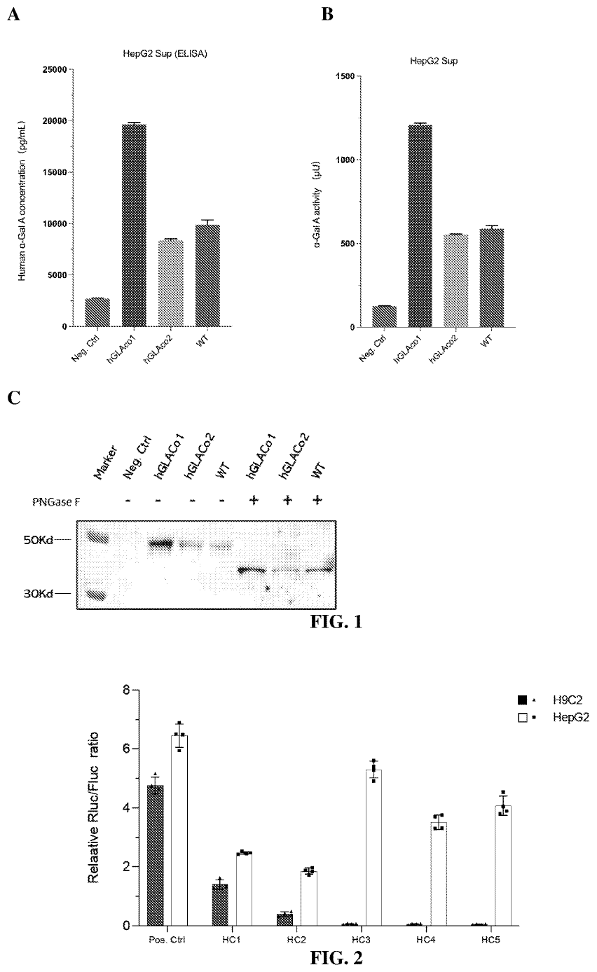

Two versions of codon-optirnized, wildtype (WT) human GLA encoding sequences (hGLAco1 and hGLAco2, having nucleotide sequences of SEQ ID NO:1 and SEQ ID NO:2, respectively) were synthesized and cloned to expression cassette backbone with chicken-beta actin (CB) promoter and bovine growth hormone (bGH) poly A sequences. The HepG2 cells were seeded into 48-well plate (1e5 cells/well). About 20 hours after seeding. 500 ng plasmids expressing hGLA or GFP (Negative control, Neg. ctrl) were transfected using Lipofectamine 3000. Seventy-two hours after transfection, the supernatants were collected and centrifuged for 10 min at 2,000 rpm. The samples for GLA were diluted by 100 or 1000 times and the undiluted samples from GFP group were used for ELISA (FIG. 1A) using human ax-galactosidase A ELISA pair set kit (Cat. No. SEK12078). The samples were diluted by 10 times for a.-galactosidase A activity (FIG. 1B) using kit (ab239716). Ten microliters of samples treated with or without PNGase F were loaded for western blot analysis (FIG. 1C) using anti-alpha Galactosidase polyclonal antibody (1:1000, PA5-27349, Thermo Fisher). The pattern after PNGase F treatment also demonstrated the proper glycosylation of α-galactosidase A expressed from HepG2 cells. The hGLAco 1 showed 2-fold higher protein expression compared to wild-type GLA. Therefore the hGLAco1 (SEQ ID NO. 1) was selected for further studies.

### 2. Hepatocyte-Cardiomyocyte (HQ) Bi-Specific Promoters In Vitro Testing.

Five promoters (HC1-HC5, having nucleotide sequences of SEQ ID NOs:4-8, respectively) containing a fixed 59-bp hepatocyte promoter (SEQ ID NO. 3) linked with various cardiomyocyte-specific transcription factor binding sites (C1-C5) were cloned upstream of Renilla luciferase (RLuc) reporter. Another reported gene, Firely luciferase (FLuc) driven by HSV-TK promoter was on the same construct and used as internal control. Rat cardiomyocyte cell line H9c2 (ATCC) and HepG2 cells were seeded in 96-well plates. One day after seeding, plasmids containing promoter HC1-HC5 were transfected using Lipofectamine 3000. Two days after transfection, dual luciferase assay kit (Cat. No. E1500, Promega) and SpectraMax i3× Multi-Mode Microplate Reader with SpectraMax Injector Cartridge were used for luminescence quantification following the manufacturer's guidelines. The RLuc/FLuc ratio was used for measuring the HC promoter strength (FIG. 2). The SV40 promoter-driven RLuc and HSV-TK promoter-driven FLuc plasmid was used as positive control (Pos. Ctrl). The HCl promoter (SEQ ID NO. 4) showed strong gene expression in both HepG2 and H9c2 (FIG. 2). It was selected for in vivo study.

### 3. Truncated Mouse Muscle Creatine Kinase (MCK) Promoter In Vitro Testing.

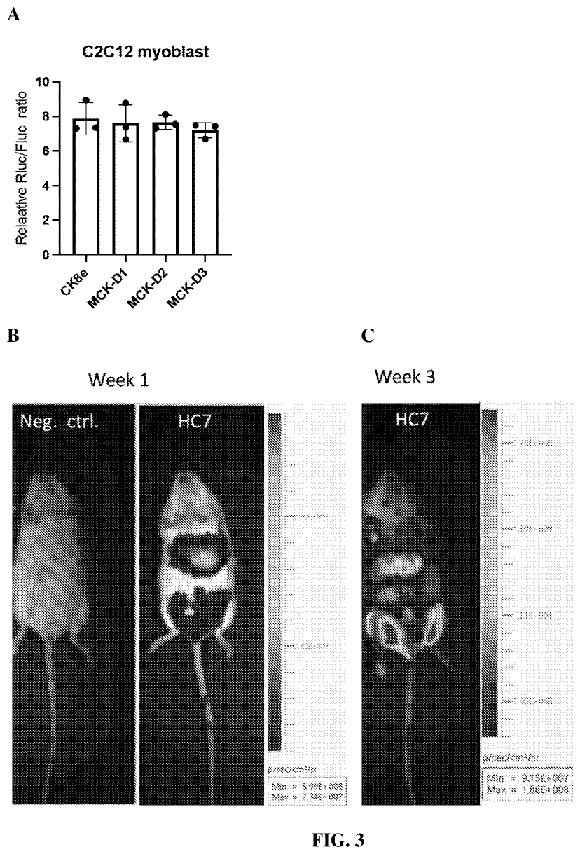

Three versions of MCK promoter (MCK-D1-MCK-D3, having nucleotide sequences of SEQ ID NOs:1 1-13, respectively) with 50-bp, 55-bp and 105-bp truncation from CK8e promoter (SEQ ID NO:14) were cloned upstream of Renilla luciferase (RLuc) reporter. C2C12 myoblast cells were seeded in 96-well plates. One day after seeding, plasmids containing MCK promoters were transfected using Lipofectamine 3000. Two days after transfection, dual luciferase assay kit (Cat. No. E1500, Promega) and SpectraMax i3× Multi-Mode Microplate Reader with SpectraMax Injector Cartridge were used for luminescence quantification. The RLuc/FLuc ratio was used for measuring the promoter strength (FIG. 3A). The CK8e promoter-driven RLuc and HSV-TK promoter-driven FLuc plasmid was used as a control. The 50-bp, 55-bp and 105-bp truncation did not result in reduced activity compared to the CK8e promoter (FIG. 3A). The MCK-D3 promoter (SEQ ID NO. 13) was selected for further in vivo study. A synthetic promoter HC7 (SEQ ID NO. 10) was constructed by combining the 59-bp hepatocyte promoter and the MCK-D3 promoter. The activity of promoter HC7 was evaluated by in vivo imaging system (IVIS). Six-week-old BALB/c male mice were intravenously (tail vein) injected with AAV (1E11 vg/mouse) containing firefly luciferase transgene driven by the HC7 promoter. The strong luminescence signals in the liver and skeletal muscles were observed one week (FIG. 3B) and three weeks (FIG. 3C) after injection.

### 4. Human GLA Expression Cassettes Mediated α-Galactosidase a Expression and Activity in HepG2.

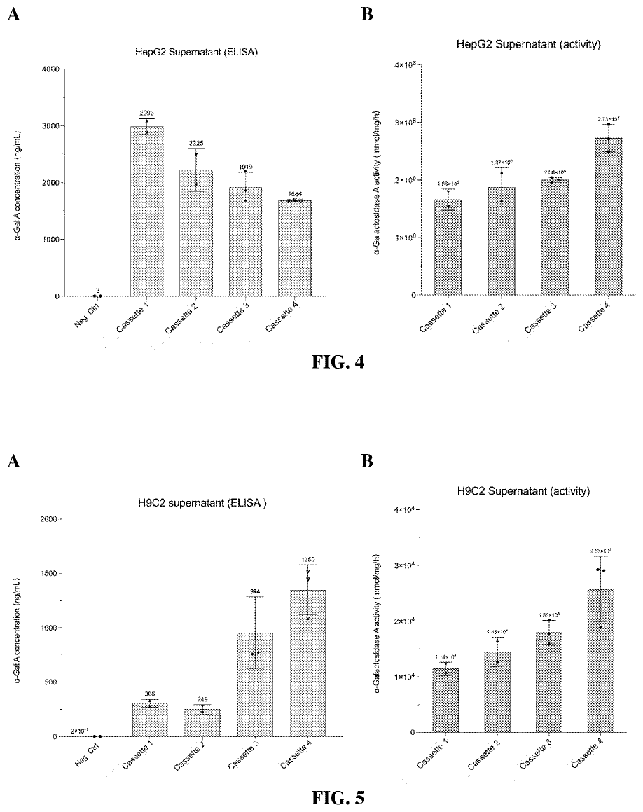

Four constructs (Table 2) with either different promoters or GLA variants were cloned to self-complementary adeno-associated virus (scAAV) vector backbone. The HepG2 cells were seeded into 48-well plate (1e5 cells/well). One day after seeding, 500 ng plasmids containing hGLA cassettes (Table 2) or GFP (Negative control, Neg. ctrl) were transfected using Lipofectamine 3000. Promoter HC6 (SEQ ID NO. 9) was constructed by combining the 59-bp hepatocyte promoter and a truncated and modified human MCK promoter (SEQ ID NO:15). Three days after transfection, the supernatants were collected and centrifuged for 10 min at 2,000 rpm. The samples for GLA were diluted by 100 or 1000 times and the undiluted samples from GFP group were used for ELISA (FIG. 4A) using human α-galactosidase A ELISA pair set kit (Cat. No. SEK12078). The samples were diluted by 10 times for α-galactosidase A activity (FIG. 4B) using kit (ab239716). thGLAco1: truncated human α-galactosidase A with 2 amino acids deletion at C-terminus (nucleotide sequence is set forth in SEQ ID NO:20). All constructs showed high α-galactosidase A protein expression and activity in HepG2.

TABLE 2
Human GLA expression cassette constructs.

| Cassette name | Promoter | GLA | polyA | SEQ ID NO. |
|---|---|---|---|---|
| Cassette 1 | HC1 | hGLAco1 | bGH | SEQ ID NO. 16 |
| Cassette 2 | HC6 | hGLAco1 | bGH | SEQ ID NO. 17 |
| Cassette 3 | HC7 | hGLAco1 | bGH | SEQ ID NO. 18 |
| Cassette 4 | HC7 | thGLAco1 | bGH | SEQ ID NO. 19 |

### 5. Human GLA Expression Cassettes Mediated α-Galactosidase a Expression and Activity in H9c2 Cells.

Four constructs (Cassettes 1-4) were transfected to H9c2 cells using Lipofectamine 3000. A GFP-expressing plasmid was used as negative control. Three days after transfection, the supernatants were collected for human α-galactosidase A ELISA (FIG. 5A) and activity (FIG. 5B) analysis. Construct Cassette 3 showed 3.1-fold, 3.8-fold expression of Cassette 1 and Cassette 2, respectively (FIG. 5A). The last two amino acids deletion resulted in 1.4-fold α-galactosidase A activity in vitro (FIG. 5B).

### 6. Characterization of Cassette 1-Mediated Activity and Efficacy in Fabry Mouse Model.

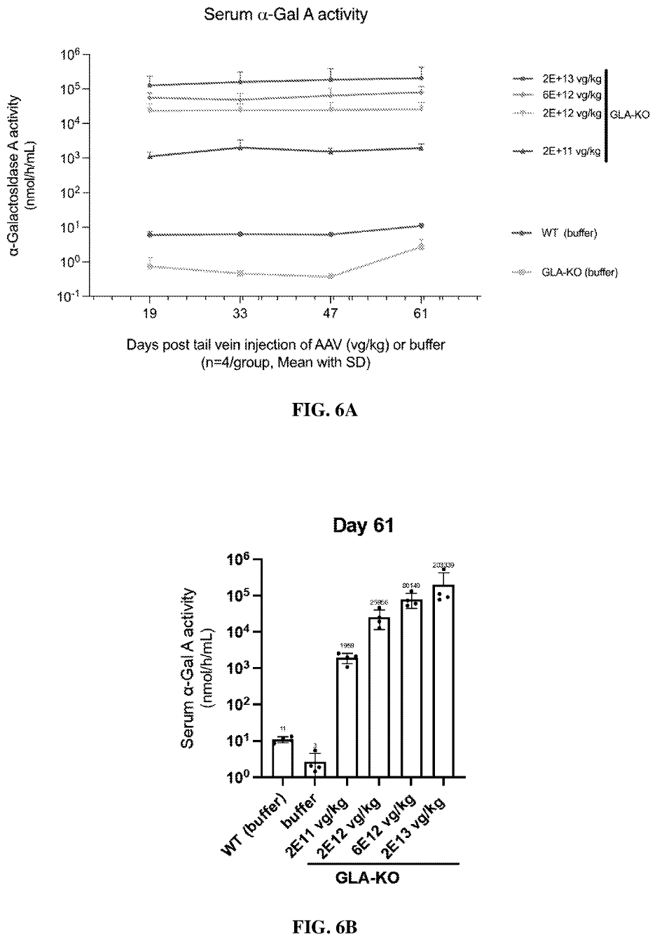

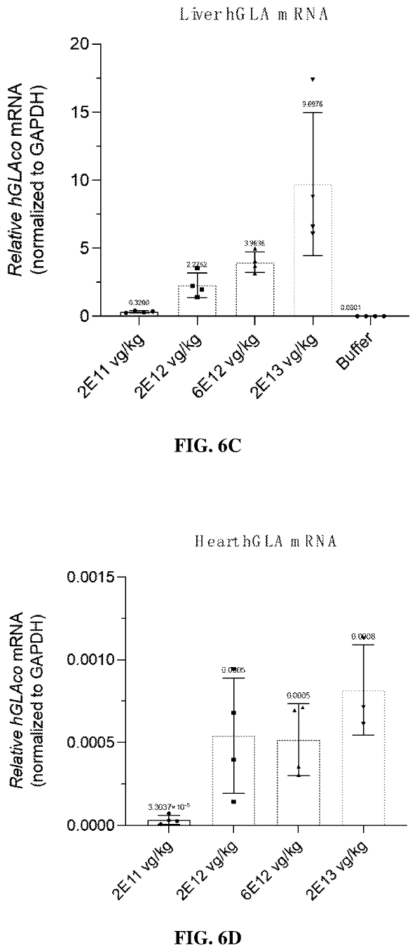

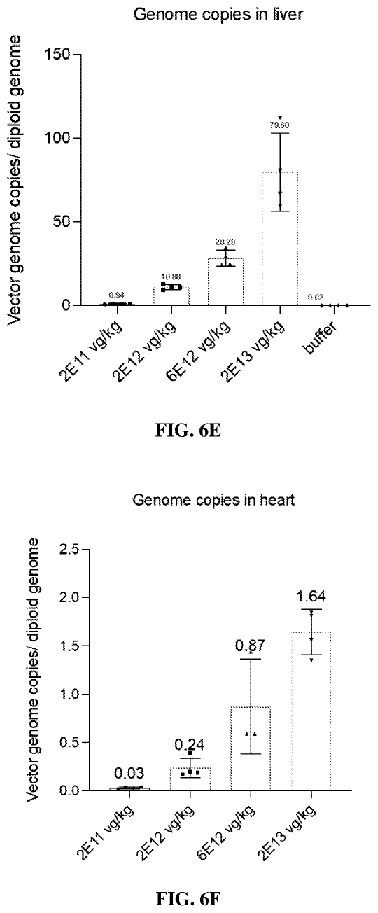

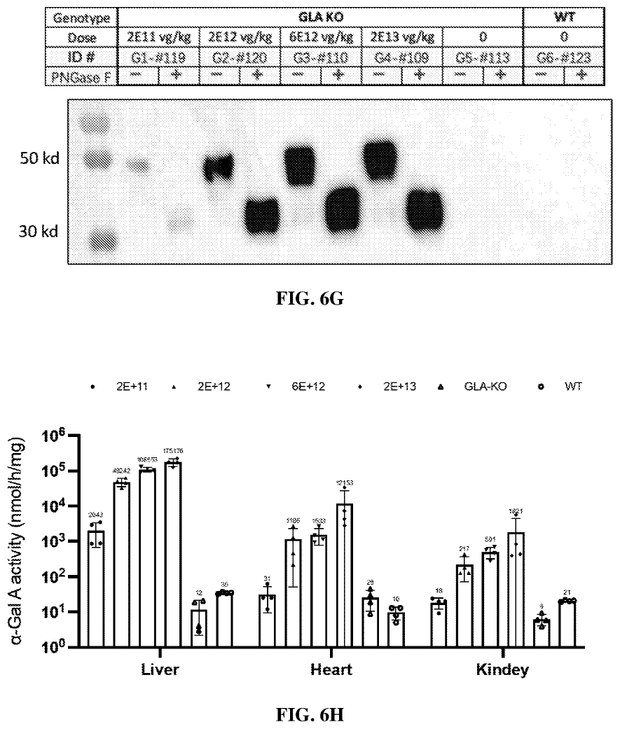

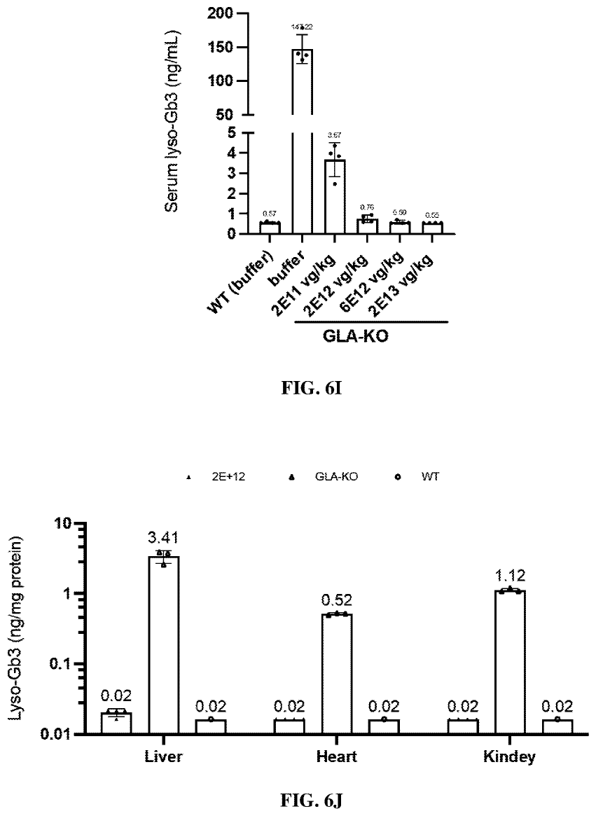

The expression cassette Cassette 1 was packaged to scAAV9. The scAAV9 was produced by triple-plasmid transfection and purified by iodixanol gradient ultracentrifugation method. The vector genome titer was measured by ddPCR and purity was confirmed by SDS-PAGE. The scAAV9 vectors were filtered through 0.22 μm and adjusted to ~1E13 vg/mL in phosphate-buffered saline (PBS) with 0.001% Pluronic F68 (formulation buffer). Four groups of adult male GLA-KO mice (JAX stock #003535) were intravenously injected through tail vein with 4 doses (2E11 vg/kg, 2E12 vg/kg, 6E12 vg/kg, 2E13 vg/kg) of scAAV9 containing the Cassette 1 expression cassette. Two groups of GLA-KO and wildtype mice were intravenously injected with formulation buffer as controls. Sera were collected by using K2-EDTA tubes biweekly for α-galactosidase A activity analysis. Stable expression of α-Gal A in serum sustained until the end of the mouse study (FIG. 6A). At day 61, the GLA-KO mice received scAAV9-Cassette 1 treatments showed 178-fold, 2360-fold, 7285-fold, and 18485-fold human α-Gal A activity in the sera at doses of 2E11 vg/kg, 2E12 vg/kg, 6E12 vg/kg, and 2E13 vg/kg, respectively, compared to wild type mice treated with buffer (FIG. 6B). At the endpoint, the mice were sacrificed and perfused with cold PBS. The total RNA was isolated from liver and heart. The DNase I-treated total RNA was used for reverse transcription (RT). The converted cDNA was used for quantitative real-time PCR using hGLAco1-specific primers/probe. The mouse GAPDH primers/probe were used as a reference for normalization. Obviously, the hGLA transgene mRNA level showed a dose-dependent effect in the liver (FIG. 6C). Higher hGLA transgene mRNA level was observed in high dose groups (2E12 vg/kg, 6E12 vg/kg, 2E13 vg/kg) compared to low dose group (2E1 I vg/kg) in the heart (FIG. 6D). The genomic DNA was isolated from liver and heart using DNeasy Blood & Tissue kit (Qiagen). The AAV vector genome copies in those tissues were measured by droplet digital PCR (ddPCR) with hGLAco1 and mouse TFRC primers/probes. The vector genome copies showed dose-dependent manner in both liver (FIG. 6E) and heart (FIG. 6F). About 15 mg liver samples were lysed with RIPA buffer. The supernatants after centrifugation were treated with or without PNGase F. For western blotting, 2 μg protein per sample was loaded and anti-alpha galactosidase polyclonal antibody (1:2000, PA5-27349, Thermo Fisher) was used. The western-blot results showed robust human α-Gal A protein expression and proper glycosylation in the liver cells after scAAV9-Cassette 1 treatment (FIG. 6G). No human α-Gal A protein was detected in the buffer-treated GLA-KO and wildtype mice. The lysed samples from liver, heart and kidney were used for α-Gal A activity analysis (FIG. 6H). Compared to buffer-treated wildtype mice, the GLA-KO mice received the lowest dose (2E 11 vg/kg) of scAAV9-Cassette 1 showed 58-fold, 3-fold, and 0.8-fold of α-Gal A activity in the liver, heart and kidney, respectively. The GLA-KO mice treated with 2E12 vg/kg of scAAV9-Cassette 1 showed 1378-fold, 119-fold, and 10-fold of α-Gal A activity in the liver, heart and kidney, respectively. The GLA-KO mice treated with 6E12 vg/kg of scAAV9-Cassette 1 showed 3104-fold, 153-fold, and 24-fold of α-Gal A activity in the liver, heart and kidney, respectively. The GLA-KO mice treated with 2E13 vg/kg of scAAV9-Cassette 1 showed 5005-fold, 1215-fold, and 87-fold of α-Gal A activity in the liver, heart and kidney, respectively.

As lyso-Gb3 is a biomarker for Fabry disease, the liquid chromatography-tandem mass spectrometry (LC-MS/MS) was used to quantify the substrate level. Compared to GLA-KO mice treated with vehicle control, all the AAV-treated GLA-KO mice showed significantly reduced lyso-Gb3 level in the serum (FIG. 6I). The 2E1 I vg/kg of scAAV9-Cassette 1 treatment reduced the lyso-Gb3 to 2.5% of that of GLA-KO control mice. Three higher doses (2E12, 6E12 and 2E13 vg/kg) of treatment completely normalized the lyso-Gb3 level in the serum (FIG. 6I). The 2E12 vg/kg of scAAV9-Cassette 1 treatment completely normalized the lyso-Gb3 level in the liver, heart and kidney (FIG. 6J).

### 7. Characterization of AAV-hGLA Mediated Activity and Efficacy in Fabry Mouse Model.

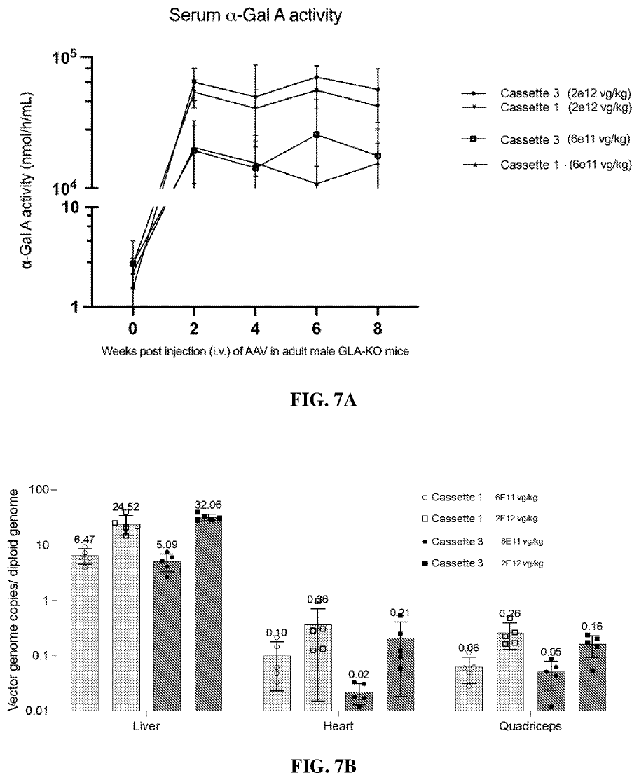

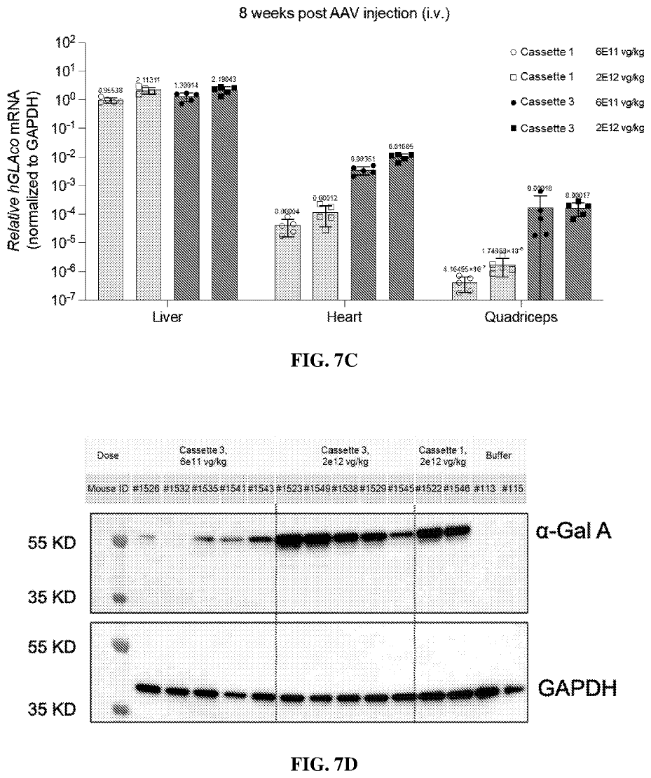

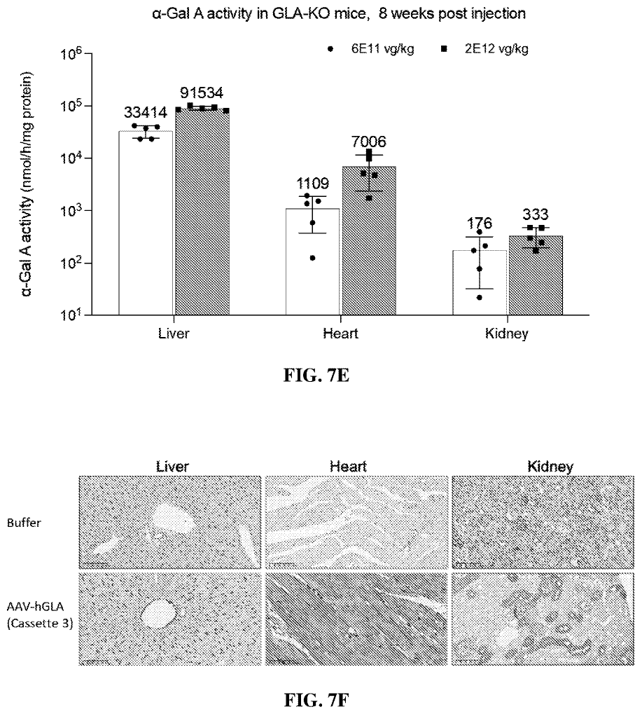

Two expression cassettes (Cassette 1 and Cassette 3) were packaged an AAV9-derived hepatic-muscle tropic variant AVT908. The AAV production, purification and characterization was similar to what is described above. Four groups of adult male GLA-KO mice (JAX stock #003535) were intravenously injected through tail vein with 2 doses (6E11 vg/kg, 2E12 vg/kg) for each hGLA expression cassette (n=5 per group). Sera were collected by using K2-EDTA tubes biweekly for α-galactosidase A activity measurements (FIG. 7A). Eight weeks after treatment, Cassette 3 showed 134% α-Gal A activity of Cassette 1 in the serum. Similar hGAL transgene mRNA level was observed in the liver for both expression cassettes. With similar or even lower vector genome copies in the muscle tissues (FIG. 7B), Cassette 3 showed significant higher hGLA transgene mRNA level in the heart and quadriceps than Cassette 1 did (FIG. 7C). Consistently, Cassette 3 showed 84-fold activity in the heart for both doses compared to Cassette 1. The RT-qPCR data confirmed the activity of hepatocyte-muscle bi-specific promoter HC7 in both liver and muscle. As the mice were totally perfused with PBS before tissue collection, the α-Gal A protein detected in the heart was derived from cardiomyocytes and/or was uptaken by them. High dose of Cassette 3 resulted in higher α-Gal A protein presence in the heart by western-blotting (FIG. 7D). The dose of 2E12 vg/kg Cassette 1 also showed the presence of α-Gal A protein in the heart. The mouse GAPDH was used as control. The liver, heart and kidney tissues were lysed for α-Gal A activity measurements. All the tissues showed high activity after AAV-hGLA treatment. The 2E12 vg/kg of Cassette 3 dosing showed 274%, 632%, 189% α-Gal A activity of its 6E11 vg/kg dosing in the liver, heart and kidney, respectively (FIG. 7E). Compared to buffer-treated GLA-KO mice, robust and broad of expressed human α-Gal A protein was detected by immunostaining in the liver and heart of GLA-KO mice treated with 2E12 vg/kg of Cassette 3 in the context of AAV (FIG. 7F). The presence of AAV-expressed human α-Gal A protein was also observed in the kidney (FIG. 7F).

### Example 2: Correction of Substrate Level in the Mouse Model of Fabry Disease by Intravenous Administration of AAV-GLA

Fabry disease (FD) is a rare X-linked metabolic disorder caused by deficiency of lysosomal enzyme α-galactosidase A (α-Gal A) due to pathogenic mutations in the GLA gene. The progressive accumulation of substrates within lysosomes results in cellular dysfunction and multiple organ damage. The current enzyme replacement therapy is an effective treatment for FD, however, the bi-weekly life-long infusion is a significant burden for patients. To provide a single administration with long-lasting effects, this study aimed to develop a recombinant adeno-associated virus (rAAV) vector-based gene therapy for FD. The codon-optimized human GLA (hGLA) cDNA with minimal CpG was generated with two algorithms. A series of synthetic hepatotropic promoters were evaluated in vitro. An optimized transgene expression cassette was then selected and packaged into AAV9 (scAAV9-hGLA). A single intravenous injection of scAAV9-hGLA to 10-week-old α-Gal A KO male mice resulted in dose-dependent α-Gal A activity in the serum, liver, heart and kidney. Stable expression of α-Gal A in serum sustained until the end of the 8-week mouse study. Compared to wild type mice treated with vehicle control (buffer), the GLA-KO mice received the lowest dose (2×10¹¹ vg/kg) showed 178-fold (×), 58×, 3×, 0.8× of α-Gal A activity in serum, liver, heart and kidney, respectively. The GLA-KO mice treated with 2×10¹² vg/kg of scAAV9-hGLA showed 2360×, 1378×, 119×, 10× of α-Gal A activity. The highest dose (2×10¹³ vg/kg) resulted in up to 18,485× of α-Gal A activity in the serum. As lyso-Gb3 is a biomarker for FD, liquid chromatography-tandem mass spectrometry (LC-MS/MS) was used to quantify the substrate level. Compared to GLA-KO mice treated with vehicle control, all the AAV-treated GLA-KO mice showed significantly reduced lyso-Gb3 level in the serum. The 2×10¹¹ vg/kg of scAAV9-hGLA treatment reduced the lyso-Gb3 to 2.5% of that of GLA-KO control mice. Three higher doses (2×10¹², 6×10¹² and 2×10¹³ vg/kg) of treatment completely normalized the lyso-Gb3 level in the serum, liver, heart and kidneys. The codon-optimized hGLA transgene mRNA level correlated with AAV vector genome copies in the liver and showed a dose-dependent effect. Liver-derived robust human α-Gal A protein expression and proper glycosylation was demonstrated by western-blot. The immunostaining confirmed the presence of α-Gal A in the liver, heart and kidney. Histopathologic examination showed no observed toxicity with all the doses. Finally, the study demonstrated that the effective dose can be further minimized by using an in-house developed novel liver-muscle tropic capsid AVT908 and promoter. The data support development of low-dose AAV-GLA gene therapy for Fabry disease.

### Example 3: Engineered AAV Capsids Mediated Robust hGLA Expression in NHPs

The human GLA transgene expression cassette 3 (SEQ ID NO:18) was packaged in capsids AAV9 (SEQ ID NO: 21), AVT917 (SEQ ID NO: 56) and AVT918 (SEQ ID NO: 58). All the 3 AAVs were produced in HEK293 cells by triple transfection method. After purification with affinity chromatography and filtration through 0.22 μm, the vector genome titer was determined by droplet digital PCR (ddPCR) using hGLA primers/probe. The purity was analyzed by SDS-PAGE. The AAV products with endotoxin level below 1 EU/mL were used in the non-human primate (NHP) studies.

Three adult (>3-year-old) male Cynomolgus monkeys without pre-existing neutralizing antibody against AAV9 were used to compare different AAV capsid mediated hGLA expression in vivo. Each animal was intravenously administrated with a single AAV expressing hGLA at the dose of 2E13 vg/kg without immunosuppressants.

The animal injected with AVT918 containing hGLA expression cassette 3 was sacrificed eight weeks after administration. The plasma, liver, heart, kidney, and skeletal muscles were harvested and processed for α-Gal A activity measurements.

The animal injected with AVT917 containing hGLA expression cassette 3 was sacrificed 13 weeks after the administration. The plasma, liver, heart, kidney and skeletal muscles were harvested and processed for α-Gal A activity measurements. The total genomic DNA from major tissues was extracted by using DNeasy Blood & Tissue kit (Qiagen, 69506). The AAV vector genome copies in the tissues were measured by ddPCR (Bio-Rad, QX200) with transgene hGLA specific primers/probe (Forward primer: Forward: 5′-GGCAGCTTTGGCTACTATGA-3′, Reverse: 5′-GTCACAGTAGCAGCCATCAA-3′, Probe: 5′-FAM-TTGATGCCCAGACCTTTGCTGACT-BHQ1-3′). The RNase P primers/probe (Thermo Fisher Scientific, 4403326) was used as reference. To quantify the transgene mRNA levels in the tissues, the total RNAs were isolated by using TaKaRa MiniBEST Universal RNA Extraction Kit (Takara, 9767). The cDNA was generated by using PrimeScript™ RT reagent Kit with gDNA Eraser (Takara, RR047B). The hGLA primers/probe were used for transgene specific transcripts. The macaque GAPDH (Forward: 5′-GCACCGTGAAGGCTGAGAAC-3′, Reverse: 5′-GGATCTCGCTCCTGGAAGATG-3′, probe: 5′-HEX-CTCGTCATCAATGGAAGCCCCATCA-BHQ1-3′) was used as housekeeping gene. Relative transgene mRNA expression level was calculated using the 2^{-Δct} method.

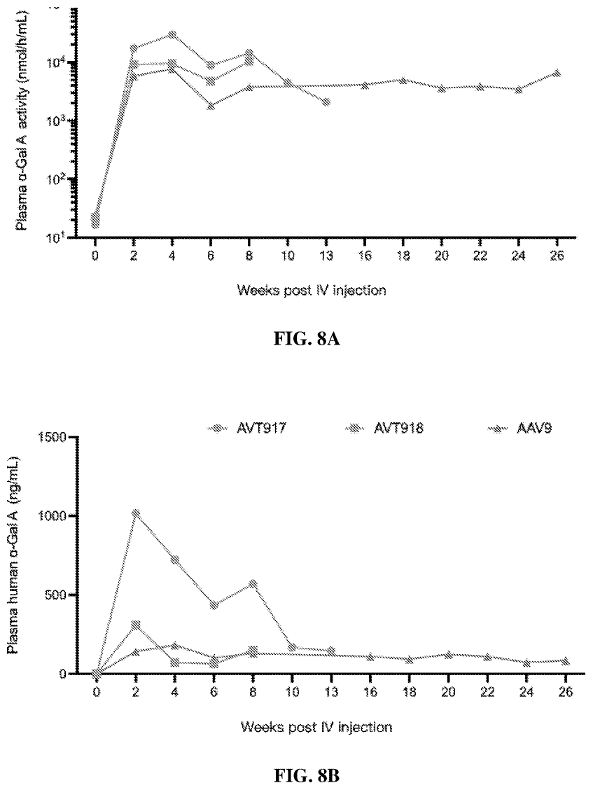

The animal injected with AAV9 containing hGLA expression cassette 3 was released after 26-week monitoring the plasma α-Gal A activity. The plasma α-Gal A activity and human α-Gal A protein levels in this animal were stable since Week 6 (triangle symbol in FIG. 8). The 26-week long-term data suggested that HC7 promoter (SEQ ID NO:10) had the stable activity in the context of AAV in NHP.

The plasma and serum samples were collected at different time points. The α-Gal A activity in the plasma was measured using the fluorescence-based enzyme activity assay. Briefly, the 4-Methylumbelliferone (4-MU) (Sigma-Aldrich, M1381-10OG ) was dissolved in DMSO and serially diluted as standard. The 4-MU-α-D-Galactopyranoside (4-MU-α-Gal) (Cayman Chemical, 16551) was used as substrate. The N-ace-tylgalactosamine (Sigma-Aldrich, A2795-500MG), an α-galactosidase B inhibitor, was also added to samples. After 2-hour incubation at 37° C. at 100 rpm shaking in the 96-well black plate (Coming, 3603), 0.1 mL of 0.5M Glycine-NaOH buffer (pH 10.5) was added to each well to stop the reaction. The fluorescence intensity (Ex/Em=365/450 nm) was measured using an end-point setting. The α-Gal A activity was calculated according to the 4MU standard curve and expressed as nmol/h/mL plasma or nmol/h/mg protein.

All the 3 capsids (AAV9, AVT917 and AVT918) mediated high α-Gal A activity level in the plasma of NHPs after intravenously administration (FIG. 8A). AVT917 mediated highest α-Gal A activity in the plasma and showed up to 1,778-fold over baseline level (Table ).

TABLE 3
Plasma α-Gal A activity (nmol/h/mL)
in the NHPs treated with AAV-hGLA.

| Week | AVT917 | AVT918 | AAV9 |
|---|---|---|---|
| 0 | 16.7 | 22.0 | 24.7 |
| 2 | 17328.1 | 9221.1 | 5775.4 |
| 4 | 29735.2 | 9528.6 | 7685.7 |
| 6 | 8899.1 | 4721.7 | 1817.4 |
| 8 | 14360.0 | 10272.2 | 3784.1 |
| 10 | 4478.4 | / | ND |
| 13 | 2082.7 | / | ND |
| 16 | / | / | 4110.8 |
| 18 | / | / | 5018.3 |
| 20 | / | / | 3620.8 |
| 22 | / | / | 3845.7 |
| 24 | / | / | 3475.7 |
| 26 | / | / | 6675.6 |

/: Not applicable;
ND: Not determined.

All the 3 capsids (AAV9, AVT917 and AVT918) mediated specific human α-Gal A protein expression in the plasma of NHPs after intravenously administration (FIG. 8B). AVT917 showed highest GLA expression according to the human α-Gal A protein concentration in the plasma (Table ).

TABLE 4
AAV-mediated hGLA expression (ng/mL) in the
NHPs after intravenously administration.

| Weeks post | AAV capsids | | |
|---|---|---|---|
| IV | AVT917 | AVT918 | AAV9 |
| 0 | 0.0 | 0.0 | 0.0 |
| 2 | 1016.7 | 307.9 | 142.7 |
| 4 | 722.3 | 71.3 | 182.2 |
| 6 | 434.2 | 63.7 | 101.0 |
| 8 | 569.9 | 149.0 | 129.9 |
| 10 | 167.1 | / | ND |
| 13 | 146.1 | / | ND |
| 16 | / | / | 110.0 |
| 18 | / | / | 92.7 |
| 20 | / | / | 123.5 |
| 22 | / | / | 109.8 |
| 24 | / | / | 71.9 |
| 26 | / | / | 83.7 |

/: Not applicable;
ND: Not determined.

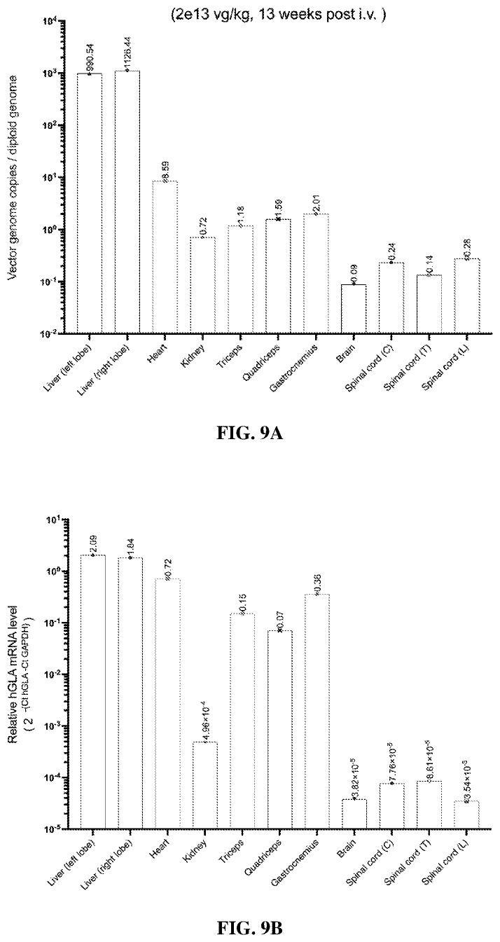

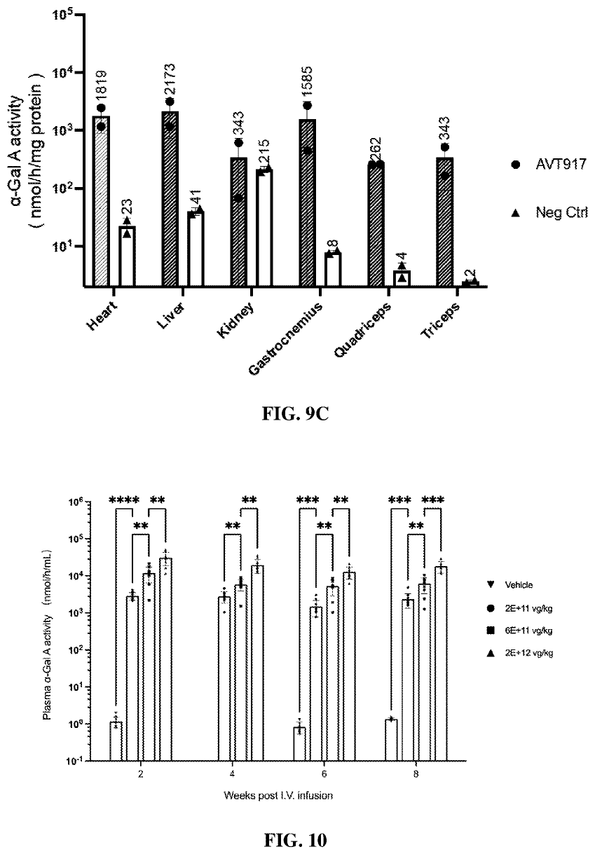

The AAV vector genome (vg) copies in the tissues of NHP treated with AVT917 was measured by ddPCR. The vector genome copies in the left lobe of liver, right lobe of liver, heart, kidney, triceps, quadriceps, gastroenemius were 990, 1126, 8.6, 0.7, 1.2, 1.6, 2.0 vg/diploid genome (dpg), respectively. The vector genome copies in the tissues of central nervous system (CNS) were below 1 vg/dpg (FIG. 9A). HC7 promoter (SEQ ID NO: 10)-driven transgene hGLA mRNA levels and tissue specificity were assessed by RT-qPCR. HC7 promoter showed robust activity in the liver, followed by heart and skeletal muscles (FIG. 9B). Weak promoter activity was observed in the kidney and tissues of CNS. The AVT917 capsid and HC7 promoter mediated high hGLA transgene expression and high level of α-Gal A activity was monitored in the plasma. Thirteen weeks after administration of AVT917 containing HC7-hGLA expression cassette 3, major tissues were harvested and processed for α-Gal A activity. Compared to the α-Gal A activities in the tissues from non-AAV-hGLA treated animal (Neg Ctrl), about 80×, 54×, 1.6×, 197×, 68×, 138× improvements were observed in the heart, liver, kidney, gastroenemius, quadriceps, and triceps, respectively (FIG. 9C).Example 4: Capsid AVT917 containing HC7-hGLA mediated efficacy in Fabry mice.

The human GLA transgene expression cassette 3 (SEQ ID NO:18) was packaged in capsid AVT917 (SEQ ID NO: 56) and produced in HEK293 cells by triple transfection method. The purified AAV prep was designated as AVT917-HC7-hGLA. To explore the minimum effective dose, adult (~16-week-old) male Fabry mice (Gla-KO) were intravenously injected with vehicle (formulation buffer) or AVT919-HC7-hGLA (cassette 3) at 3 doses (2E11 vg/kg, 6E11 vg/kg, 2E12 vg/kg, n=10/group). The plasma was collected bi-weekly for α-Gal A activity measurements. Compared to the vehicle group mice, all the mice treated with AVT917-HC7-hGLA showed supraphysiological α-Gal A activities in the plasma during the 8-week study (FIG. 10A). A dose-dependent increase of α-Gal A activity was observed among the groups with treatment of AVT917-HC7-hGLA (FIG. 10A). Eight weeks after the treatment, the mean plasma α-Gal A activities in the vehicle, low dose (2E11 vg/kg), mid dose (6E11 vg/kg), high dose (2E13 vg/kg) group mice were 1.37 nmol/h/mL, 2336.19 nmol/h/mL, 6140.66 nmol/h/mL, 18189.11 nmol/h/mL, respectively, corresponding to 1×, 1710×, 4495×, 13314× improvements over the mean plasma α-Gal A activity levels in the vehicle-treated Fabry mice.

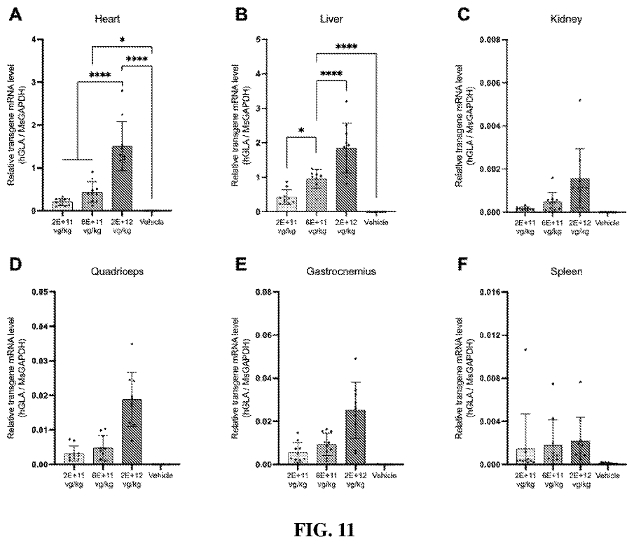

The transgene hGLA mRNA levels in the tissues of Fabry mice treated with AVT917-HC7-hGLA were assessed by RT-qPCR. Primarily, the hGLA transcripts were in the liver and heart (FIG. 11A,B). Weak hGLA mRNA levels were in the quadriceps and gastroenemius skeletal muscles (FIGS. 11D, E). Negligible transgene expression was observed in the kidney and spleen (FIG. 11C, F). These data supported that HC7 promoter has the hepatocyte-myocyte bi-specific activity in vivo.

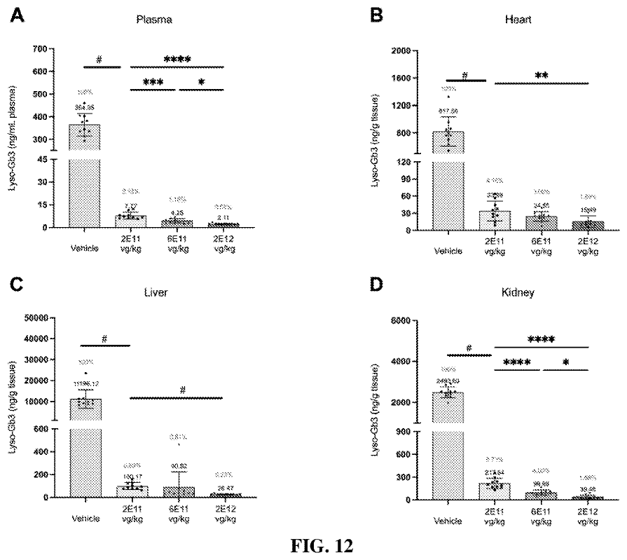

Eight weeks after a single intravenous dosing of AVT917-HC7-hGLA in the Fabry mice, the key tissues were harvested and processed for substrate Lyso-Gb3 measurements using Liquid Chromatography with tandem mass spectrometry (LC-MS-MS). Compared to vehicle-treated Fabry mice, all the Fabry mice treated with AVT917-HC7-hGLA showed significant substrate reduction in the plasma, liver, heart and kidney (FIG. 12). The Fabry mice treated with low dose (2E11 vg/kg) of AVT917-HC7-hGLA had only 2.13%, 4.16%, 0.89%, 8.73% of Lyso-Gb3 remaining in the plasma, liver, heart, and kidney respectively, compared with vehicle-treated Fabry mice (FIG. 12). The Lyso-Gb3 level in the tissues of vehicle-treated Fabry mice was set as 100%.

### Example 4: AVT917-Mediated hGLA Transgene Expression in NHPs

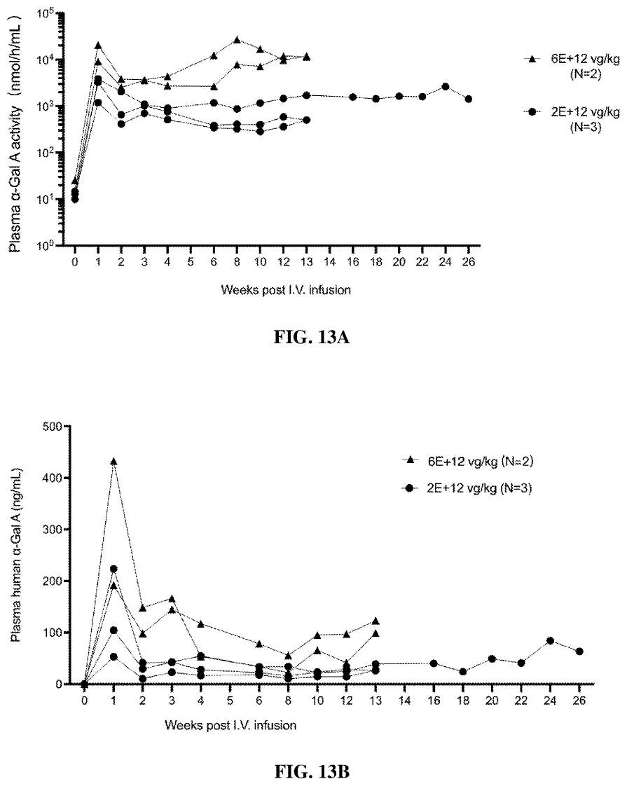

Adult male Cynomolgus monkeys were intravenously administrated with AVT917-HC7-hGLA at the dose of 2E12 vg/kg (N=3) and 6E12 vg/kg (N=2). The plasma was collected for α-Gal A activity measurements by using fluorescence-based 4-MU method. The mean α-Gal A activities from the 5 animals before AAV administration was 15.6 nmol/h/L. One week after the administration, 2E12 vg/kg and 6E12 vg/kg resulted in 234×, 711× improvements of α-Gal A activity over the baseline level, respectively. The α-Gal A activity in the plasma of 3 animals treated with 2E12 vg/kg was stable since Week 6 (FIG. 13A). One of the animals was observed as long as 26 weeks. The α-Gal A activity in the plasma of 2 animals treated with 6E12 vg/kg was stable between Week 2 and Week 6 (FIG. 13A). The enzyme activity increased slightly after Week 6 (FIG. 13A). These data supported that AVT917-HC7-hGLA with low dose could provide supraphysiological and stable α-Gal A activity in the NHPs after a single intravenous administration.

The human α-Gal A protein expressed from the NHPs treated with AVT917-HC7-hGLA was measured by ELISA. Before the administration, no human α-Gal A protein was detected in the plasma. One week after the AVT917-HC7-hGLA treatment, the 3 animals with the dose of 2E12 vg/kg showed 53 ng/mL, 105 ng/mL and 224 ng/mL of human α-Gal A in the plasma. The human α-Gal protein level was stable (mean level: 27~36 ng/mL) in those 3 animals after Week 2 (FIG. 13B). One animal showed 63 ng/mL of human α-Gal A protein in the plasma at Week 26. These data confirmed the HC7 could provide stable promoter activity in NHPs. The 2 animals treated with 6E12 vg/kg of AVT917-HC7-hGLA showed 192 ng/mL and 433 ng/mL of human α-Gal A in the plasma at Week 1. The average human α-Gal A protein levels were 123 ng/mL, 111 ng/mL at Week 2, Week 13, respectively (FIG. 13B).

### Example 5: Comparison of AVT917 and AVT919-Mediated hGLA Transgene Expression in NHPs

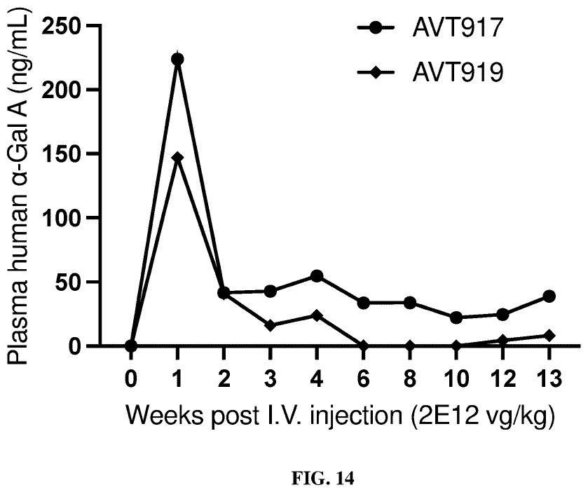

Adult male Cynomolgus monkeys were intravenously administrated with AVT917 or AVT919 containing cassette 3 (SEQ ID:18) at the dose of 2E12 vg/kg (N=1/capsid). The plasma was collected for human α-Gal A protein concentration measurements by ELISA. The level of human α-Gal A in the plasma before AAV administration was 0 ng/mL. One week after the administration, AVT917 and AVT919 resulted in 224 ng/mL, 147 ng/mL of human α-Gal A in the plasma, respectively (FIG. 14). AVT917 mediated higher plasma human α-Gal A level than AVT919 did (FIG. 14).

### INCORPORATION BY REFERENCE

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

1. A polynucleotide comprising a nucleotide sequence encoding human α-galactosidase A, wherein the nucleotide sequence encoding human α-galactosidase A comprises SEQ ID NO: 1.

2. An expression cassette comprising a polynucleotide comprising a nucleotide sequence encoding human α-galactosidase A of claim 1 and a polynucleotide comprising a nucleotide sequence of a promoter, wherein the nucleotide sequence encoding the promoter is operably linked to the nucleotide sequence encoding human α-galactosidase A.

3. The expression cassette of claim 2, wherein the nucleotide sequence of the promoter comprises SEQ ID NO:3, SEQ ID NO:4, or SEQ ID NO: 10.

4. The expression cassette of claim 2, wherein the promoter is a hepatocyte-specific promoter, or a hepatocyte-myocyte bispecific promoter; and/or wherein the expression cassette further comprises a nucleotide sequence of a polyA signal.

5-6. (canceled)

7. The expression cassette of claim 4, wherein the nucleotide sequence of the expression cassette comprises SEQ ID NO:16 or SEQ ID NO:18.

8. A polynucleotide comprising a nucleotide sequence of a promoter, wherein the nucleotide sequence of the promoter comprises SEQ ID NO:3, SEQ ID NO:4, or SEQ ID NO:10.

9. An expression cassette comprising a polynucleotide comprising a nucleotide sequence of the promoter of claim 8 and a polynucleotide comprising a nucleotide sequence encoding human α-galactosidase A, wherein the nucleotide sequence encoding the promoter is operably linked to the nucleotide sequence of human α-galactosidase A.

10. The expression cassette of claim 9, wherein the nucleotide sequence encoding human α-galactosidase A comprises SEQ ID NO: 1 and/or
wherein the promoter is a hepatocyte-specific promoter, or a hepatocyte-myocyte bispecific promoter; and/or
wherein the expression cassette further comprises a nucleotide sequence of a polyA signal.

11-13. (canceled)

14. The expression cassette of claim 10, wherein the nucleotide sequence of the expression cassette comprises SEQ ID NO:16 or SEQ ID NO:18.

15. A vector comprising the polynucleotide of claim 1.

16. A recombinant viral particle comprising a recombinant viral genome comprising the expression cassette of claim 2.

17. A recombinant adeno-associated virus (AAV) particle comprising: (a) an AAV capsid; and (b) a recombinant AAV genome comprising the expression cassette of claim 2 flanked by AAV-inverted terminal repeats (ITRs).

18. The recombinant AAV particle of claim 17, which is a recombinant AAV serotype 9 (rAAV9) particle; and/or the AAV capsid comprises a variant AAV9 capsid protein.

19. (canceled)

20. The recombinant AAV particle of claim 19, wherein the variant AAV9 capsid protein comprises the amino acid sequence of SEQ ID NO:31, 38, 49, 51, 53, 55, 57, 59 or 61; or, wherein the variant AAV9 capsid protein comprises the amino acid sequence of SEO ID NO:30, 37, 48, 50, 52, 54, 56, 58 or 60.

21. (canceled)

22. A host cell comprising the polynucleotide of claim 1.

23. A population of host cells stably transduced by the recombinant viral particle of claim 16.

24. A pharmaceutical composition comprising the recombinant viral particle of claim 16 and a pharmaceutically acceptable carrier.

25. A method of producing a recombinant viral particle or a recombinant AAV particle, comprising culturing the host cell of claim 22.

26. A method of treating a disease or disorder in a subject in need thereof, comprising administering to the subject the recombinant viral particle of claim 16.

27. The method of claim 26, wherein the disease or disorder is a lysosomal storage disorder or Fabry disease; and/or the subject is a human.

28. (canceled)
